SEQUENCE LISTING (112 sequences)

SEQ ID NO: 1 - protein, 11 aa, Artificial Sequence
  Synthetic construct
RASQEISDYLT

SEQ ID NO: 2 - protein, 7 aa, Artificial Sequence
  Synthetic construct
AASTLDS

SEQ ID NO: 3 - protein, 9 aa, Artificial Sequence
  Synthetic construct
LQYASFPLT

SEQ ID NO: 4 - protein, 107 aa, Artificial Sequence
  Synthetic construct
DIQMTQSPSSLSASLGERVSLTCRASQEISDYLTWLQQKPDGTIKRLIYAASTLDSGVPKRFSGSRSGSDYSLTISSLESEDFADYYCLQYASFPLTFGAGTKLELK

SEQ ID NO: 5 - protein, 127 aa, Artificial Sequence
  Synthetic construct
MRVPAHVFGFLLLWFPGTRCDIQMTQSPSSLSASLGERVSLTCRASQEISDYLTWLQQKPDGTIKRLIYAASTLDSGVPKRFSGSRSGSDYSLTISSLESEDFADYYCLQYASFPLTFGAGTKLELK

SEQ ID NO: 6 - DNA, 33 bp, Artificial Sequence
  Synthetic construct
cgggcaagtcaggaaattagtgattacttaacc

SEQ ID NO: 7 - DNA, 21 bp, Artificial Sequence
  Synthetic construct
gccgcatccactttagattct

SEQ ID NO: 8 - DNA, 27 bp, Artificial Sequence
  Synthetic construct
ctacaatatgctagttttccgctcacg

SEQ ID NO: 9 - DNA, 321 bp, Artificial Sequence
  Synthetic construct
gacatccagatgacccagtctccatcctccttatctgcctctctgggagaaagagtcagtctcacttgtcgggcaagtcaggaaattagtgattacttaacctggcttcagcagaaaccagatggaactattaaacgcctgatctacgccgcatccactttagattctggtgtcccaaaaaggttcagtggcagtaggtctgggtcagattattctctcaccatcagcagccttgagtctgaagattttgcagactattactgtctacaatatgctagttttccgctcacgttcggtgctgggactaagctggagctgaaa

SEQ ID NO: 10 - DNA, 381 bp, Artificial Sequence
  Synthetic construct
atgagggttcctgctcacgtttttggcttcttgttgctctggtttccaggtaccagatgtgacatccagatgacccagtctccatcctccttatctgcctctctgggagaaagagtcagtctcacttgtcgggcaagtcaggaaattagtgattacttaacctggcttcagcagaaaccagatggaactattaaacgcctgatctacgccgcatccactttagattctggtgtcccaaaaaggttcagtggcagtaggtctgggtcagattattctctcaccatcagcagccttgagtctgaagattttgcagactattactgtctacaatatgctagttttccgctcacgttcggtgctgggactaagctggagctgaaa

SEQ ID NO: 11 - protein, 5 aa, Artificial Sequence
  Synthetic construct
SFGMH

SEQ ID NO: 12 - protein, 17 aa, Artificial Sequence
  Synthetic construct
YVSGGSSTIYYADTVKG

SEQ ID NO: 13 - protein, 13 aa, Artificial Sequence
  Synthetic construct
SFSLLRLPVYFDF

SEQ ID NO: 14 - protein, 122 aa, Artificial Sequence
  Synthetic construct
DVQLVESGGGLVQPGGSRKLSCAASGFTFSSFGMHWVRQAPEKGLEWVAYVSGGSSTIYYADTVKGRFTISRDNPKNTLFLQMTSLRSEDTAMYYCARSFSLLRLPVYFDFWGQGTTLTVSS

SEQ ID NO: 15 - protein, 141 aa, Artificial Sequence
  Synthetic construct
MDSRLNLVFLVLILKGVQCDVQLVESGGGLVQPGGSRKLSCAASGFTFSSFGMHWVRQAPEKGLEWVAYVSGGSSTIYYADTVKGRFTISRDNPKNTLFLQMTSLRSEDTAMYYCARSFSLLRLPVYFDFWGQGTTLTVSS

SEQ ID NO: 16 - DNA, 15 bp, Artificial Sequence
  Synthetic construct
agctttggaatgcac

SEQ ID NO: 17 - DNA, 51 bp, Artificial Sequence
  Synthetic construct
tacgttagtggtggcagtagtaccatctactatgcagacacagtgaagggc

SEQ ID NO: 18 - DNA, 423 bp, Artificial Sequence
  Synthetic construct
atggactccaggctcaatttagttttccttgtccttattttaaaaggtgtccagtgtgatgtgcagctggtggagtctgggggaggcttagtgcagcctggagggtcccggaaactctcctgtgcagcctctggattcactttcagtagctttggaatgcactgggttcgtcaggctccagagaaggggctggagtgggtcgcatacgttagtggtggcagtagtaccatctactatgcagacacagtgaagggccgattcaccatctccagagacaatcccaagaacaccctgttcctgcaaatgaccagtctcaggtctgaggacacggccatgtattactgtgcaagatccttctcattactgcgactaccggtctactttgacttctggggccaaggcaccactctcacagtctcctca

SEQ ID NO: 19 - DNA, 366 bp, Artificial Sequence
  Synthetic construct
gatgtgcagctggtggagtctgggggaggcttagtgcagcctggagggtcccggaaactctcctgtgcagcctctggattcactttcagtagctttggaatgcactgggttcgtcaggctccagagaaggggctggagtgggtcgcatacgttagtggtggcagtagtaccatctactatgcagacacagtgaagggccgattcaccatctccagagacaatcccaagaacaccctgttcctgcaaatgaccagtctcaggtctgaggacacggccatgtattactgtgcaagatccttctcattactgcgactaccggtctactttgacttctggggccaaggcaccactctcacagtctcctca

SEQ ID NO: 20 - DNA, 23 bp, Artificial Sequence
  Synthetic construct
aacgatcagtaagaatacagagc

SEQ ID NO: 21 - DNA, 25 bp, Artificial Sequence
  Synthetic construct
gcactctgattgacaaatacgattt

SEQ ID NO: 22 - DNA, 21 bp, Artificial Sequence
  Synthetic construct
cgagaatttgagtcctggata

SEQ ID NO: 23 - DNA, 21 bp, Artificial Sequence
  Synthetic construct
cctactttgaaggagaaagaa

SEQ ID NO: 24 - DNA, 21 bp, Artificial Sequence
  Synthetic construct
cagaatgcactgttacctgga

SEQ ID NO: 25 - DNA, 21 bp, Artificial Sequence
  Synthetic construct
gctgatcccaattctctttca

SEQ ID NO: 26 - DNA, 22 bp, Artificial Sequence
  Synthetic construct
ccggacgacagatattacaacg

SEQ ID NO: 27 - DNA, 21 bp, Artificial Sequence
  Synthetic construct
gctgggtcaatggtgctatag

SEQ ID NO: 28 - DNA, 20 bp, Artificial Sequence
  Synthetic construct
aggggaccgtgctctttaac

SEQ ID NO: 29 - DNA, 20 bp, Artificial Sequence
  Synthetic construct
ctgagttccccactggagtc

SEQ ID NO: 30 - DNA, 18 bp, Artificial Sequence
  Synthetic construct
cagcgacaacccgtacac

SEQ ID NO: 31 - DNA, 21 bp, Artificial Sequence
  Synthetic construct
gacttggagcttgagtcctga

SEQ ID NO: 32 - DNA, 20 bp, Artificial Sequence
  Synthetic construct
tgggaaagtacacggggata

SEQ ID NO: 33 - DNA, 20 bp, Artificial Sequence
  Synthetic construct
tattgaccaagggcattcac

SEQ ID NO: 34 - DNA, 23 bp, Artificial Sequence
  Synthetic construct
ccatcggactctcataggttgtc

SEQ ID NO: 35 - DNA, 24 bp, Artificial Sequence
  Synthetic construct
gacctgtacttattgtctctcatc

SEQ ID NO: 36 - DNA, 22 bp, Artificial Sequence
  Synthetic construct
cctcatccctgtctattgctcc

SEQ ID NO: 37 - DNA, 22 bp, Artificial Sequence
  Synthetic construct
gttggctccttgcttgttctgc

SEQ ID NO: 38 - DNA, 21 bp, Artificial Sequence
  Synthetic construct
cagaatgcactgttacctgga

SEQ ID NO: 39 - DNA, 21 bp, Artificial Sequence
  Synthetic construct
gctgatcccaattctctttca

SEQ ID NO: 40 - DNA, 20 bp, Artificial Sequence
  Synthetic construct
gaccgactgagacccaacat

SEQ ID NO: 41 - DNA, 20 bp, Artificial Sequence
  Synthetic construct
tggtttggcgttgtactctg

SEQ ID NO: 42 - DNA, 20 bp, Artificial Sequence
  Synthetic construct
agcgatgacgtctctgaggt

SEQ ID NO: 43 - DNA, 20 bp, Artificial Sequence
  Synthetic construct
gtacagggacaccagccact

SEQ ID NO: 44 - DNA, 20 bp, Artificial Sequence
  Synthetic construct
agagctacgagctgcctgac

SEQ ID NO: 45 - DNA, 20 bp, Artificial Sequence
  Synthetic construct
agcactgtgttggcgtacag

SEQ ID NO: 46 - DNA, 21 bp, Artificial Sequence
  Synthetic construct
ggcattcgggaagtattgtcg

SEQ ID NO: 47 - DNA, 21 bp, Artificial Sequence
  Synthetic construct
ggtaggcgcctcagtcgtatc

SEQ ID NO: 48 - DNA, 20 bp, Artificial Sequence
  Synthetic construct
cacgtggttggaagctaacc

SEQ ID NO: 49 - DNA, 21 bp, Artificial Sequence
  Synthetic construct
gaaggccagagcataagatgc

SEQ ID NO: 50 - DNA, 20 bp, Artificial Sequence
  Synthetic construct
ggcagcagaacccttcttat

SEQ ID NO: 51 - DNA, 21 bp, Artificial Sequence
  Synthetic construct
ggccgttgctctgtattctta

SEQ ID NO: 52 - DNA, 23 bp, Artificial Sequence
  Synthetic construct
ggtgtgaaccatgagaagtatga

SEQ ID NO: 53 - DNA, 22 bp, Artificial Sequence
  Synthetic construct
gagtccttccacgataccaaag

SEQ ID NO: 54 - DNA, 23 bp, Artificial Sequence
  Synthetic construct
ccacggattacagtacctatagc

SEQ ID NO: 55 - DNA, 21 bp, Artificial Sequence
  Synthetic construct
ggctgtccataggttccatag

SEQ ID NO: 56 - DNA, 25 bp, Artificial Sequence
  Synthetic construct
ctctctctctctctctaaccatctc

SEQ ID NO: 57 - DNA, 19 bp, Artificial Sequence
  Synthetic construct
tctctgggaccagcataca

SEQ ID NO: 58 - DNA, 24 bp, Artificial Sequence
  Synthetic construct
ggaagaggttcccacggagggcat

SEQ ID NO: 59 - DNA, 20 bp, Artificial Sequence
  Synthetic construct
ccggcgacactttgtttact

SEQ ID NO: 60 - DNA, 20 bp, Artificial Sequence
  Synthetic construct
agagcctgggggtgagacat

SEQ ID NO: 61 - DNA, 23 bp, Artificial Sequence
  Synthetic construct
gcctcttcaacttaaccgcatga

SEQ ID NO: 62 - DNA, 29 bp, Artificial Sequence
  Synthetic construct
gtttgtgccttcatgggaaatggttattc

SEQ ID NO: 63 - DNA, 24 bp, Artificial Sequence
  Synthetic construct
ctagtgtcttgtgtgtccctaggg

SEQ ID NO: 64 - DNA, 20 bp, Artificial Sequence
  Synthetic construct
tcctcctgtttcatccaagc

SEQ ID NO: 65 - DNA, 22 bp, Artificial Sequence
  Synthetic construct
gaactgcggggagagcgtggtg

SEQ ID NO: 66 - DNA, 23 bp, Artificial Sequence
  Synthetic construct
tccccgagagcgagtccgaagtc

SEQ ID NO: 67 - DNA, 18 bp, Artificial Sequence
  Synthetic construct
cgaccacagcgggagtag

SEQ ID NO: 68 - DNA, 18 bp, Artificial Sequence
  Synthetic construct
gcgagaggctgggaaggt

SEQ ID NO: 69 - DNA, 20 bp, Artificial Sequence
  Synthetic construct
gaccggacaacaaagaggag

SEQ ID NO: 70 - DNA, 18 bp, Artificial Sequence
  Synthetic construct
ctccagacacgggctttc

SEQ ID NO: 71 - DNA, 18 bp, Artificial Sequence
  Synthetic construct
ccgccccgccgctcattc

SEQ ID NO: 72 - DNA, 20 bp, Artificial Sequence
  Synthetic construct
gcttccccgccctttacctc

SEQ ID NO: 73 - DNA, 23 bp, Artificial Sequence
  Synthetic construct
tatggctttatgctgtttttcaa

SEQ ID NO: 74 - DNA, 21 bp, Artificial Sequence
  Synthetic construct
ctcatcccggccgcattagac

SEQ ID NO: 75 - DNA, 19 bp, Artificial Sequence
  Synthetic construct
gtgcttcctgctcctcttc

SEQ ID NO: 76 - DNA, 21 bp, Artificial Sequence
  Synthetic construct
gctagcaagatcctccaaaca

SEQ ID NO: 77 - DNA, 20 bp, Artificial Sequence
  Synthetic construct
tcccaaagtgctgggattac

SEQ ID NO: 78 - DNA, 22 bp, Artificial Sequence
  Synthetic construct
tctcatggtttccctttctagc

SEQ ID NO: 79 - protein, 241 aa, Artificial Sequence
  Synthetic construct
GYDFCSERHNCMENSICRNLNDRAVCSCRDGFRALREDNAYCEDIDECAEGRHYCRENTMCVNTPGSFMCICKTGYIRIDDYSCTEHDECITNQHNCDENALCFNTVGGHNCVCKPGYTGNGTTCKAFCKDGCRNGGACIAANVCACPQGFTGPSCETDIDECSDGFVQCDSRANCINLPGWYHCECRDGYHDNGMFSPSGESCEDIDECGTGRHSCANDTICFNLDGGYDCRCPHGKNCT

SEQ ID NO: 80 - DNA, 423 bp, Artificial Sequence; identical to SEQ ID NO: 18
  Synthetic construct

SEQ ID NO: 81 - DNA, 423 bp, Artificial Sequence; identical to SEQ ID NO: 18
  Synthetic construct

SEQ ID NO: 82 - DNA, 422 bp, Artificial Sequence
  Synthetic construct
tggactccaggctcaatttagttttccttgtccttattttaaaaggtgtccagtgtgatgtgcagctggtggagtctgggggaggcttagtgcagcctggagggtcccggaaactctcctgtgcagcctctggattcactttcagtagctttggaatgcactgggttcgtcaggctccagagaaggggctggagtgggtcgcatacgttagtggtggcagtagtaccatctactatgcagacacagtgaagggccgattcaccatctccagagacaatcccaagaacaccctgttcctgcaaatgaccagtctcaggtctgaggacacggccatgtattactgtgcaagatccttctcattactgcgactaccggtctactttgacttctggggccaaggcaccactctcacagtctcctca

SEQ ID NO: 83 - DNA, 420 bp, Artificial Sequence
  Synthetic construct
gactccaggctcaatttagttttccttgtccttattttaaaaggtgtccagtgtgatgtgcagctggtggagtctgggggaggcttagtgcagcctggagggtcccggaaactctcctgtgcagcctctggattcactttcagtagctttggaatgcactgggttcgtcaggctccagagaaggggctggagtgggtcgcatacgttagtggtggcagtagtaccatctactatgcagacacagtgaagggccgattcaccatctccagagacaatcccaagaacaccctgttcctgcaaatgaccagtctcaggtctgaggacacggccatgtattactgtgcaagatccttctcattactgcgactaccggtctactttgacttctggggccaaggcaccactctcacagtctcctca

SEQ ID NO: 84 - DNA, 407 bp, Artificial Sequence
  Synthetic construct
atttagttttccttgtccttattttaaaaggtgtccagtgtgatgtgcagctggtggagtctgggggaggcttagtgcagcctggagggtcccggaaactctcctgtgcagcctctggattcactttcagtagctttggaatgcactgggttcgtcaggctccagagaaggggctggagtgggtcgcatacgttagtggtggcagtagtaccatctactatgcagacacagtgaagggccgattcaccatctccagagacaatcccaagaacaccctgttcctgcaaatgaccagtctcaggtctgaggacacggccatgtattactgtgcaagatccttctcattactgcgactaccggtctactttgacttctggggccaaggcaccactctcacagtctcctca

SEQ ID NO: 85 - DNA, 423 bp, Artificial Sequence; identical to SEQ ID NO: 18
  Synthetic construct

SEQ ID NO: 86 - protein, 5 aa, Artificial Sequence
  Synthetic construct
DYYLY

SEQ ID NO: 87 - protein, 17 aa, Artificial Sequence
  Synthetic construct
YISNGGDITYYPDTVKG

SEQ ID NO: 88 - protein, 15 aa, Artificial Sequence
  Synthetic construct
GRVRSYYGNPDAMDF

SEQ ID NO: 89 - protein, 124 aa, Artificial Sequence
  Synthetic construct
EVKLVESGGGLVQPGGSLKLSCATSGFTFSDYYLYWVRQTPEKRLEWVAYISNGGDITYYPDTVKGRFAISRDNAKNTLYLQMSRLKSEDTAMYYCARGRVRSYYGNPDAMDFWGQGTSVTVSS

SEQ ID NO: 90 - protein, 143 aa, Artificial Sequence
  Synthetic construct
MNLGLSLIFLVLVLKGVQCEVKLVESGGGLVQPGGSLKLSCATSGFTFSDYYLYWVRQTPEKRLEWVAYISNGGDITYYPDTVKGRFAISRDNAKNTLYLQMSRLKSEDTAMYYCARGRVRSYYGNPDAMDFWGQGTSVTVSS

SEQ ID NO: 91 - DNA, 15 bp, Artificial Sequence
  Synthetic construct
gactattacttatat

SEQ ID NO: 92 - DNA, 51 bp, Artificial Sequence
  Synthetic construct
tacattagcaatggtggtgatattacctattatccagacactgtaaagggc

SEQ ID NO: 93 - DNA, 45 bp, Artificial Sequence
  Synthetic construct
ggaagagtccgatcctactatggtaaccccgatgctatggacttc

SEQ ID NO: 94 - DNA, 429 bp, Artificial Sequence
  Synthetic construct
atgaacttggggctcagcttgattttccttgtccttgttttaaaaggtgtccagtgtgaagttaagctggtggagtctgggggaggcttagtgcagcctggagggtccctgaaactctcctgtgctacctctggattcactttcagtgactattacttatattgggttcgccagactccagagaagaggctggagtgggtcgcatacattagcaatggtggtgatattacctattatccagacactgtaaagggccgattcgccatctccagagacaatgccaagaacaccctgtacctgcaaatgagccgtctgaagtctgaggacacagccatgtattactgtgcaaggggaagagtccgatcctactatggtaaccccgatgctatggacttctggggtcaaggaacctcagtcaccgtctcctca

SEQ ID NO: 95 - DNA, 372 bp, Artificial Sequence
  Synthetic construct
gaagttaagctggtggagtctgggggaggcttagtgcagcctggagggtccctgaaactctcctgtgctacctctggattcactttcagtgactattacttatattgggttcgccagactccagagaagaggctggagtgggtcgcatacattagcaatggtggtgatattacctattatccagacactgtaaagggccgattcgccatctccagagacaatgccaagaacaccctgtacctgcaaatgagccgtctgaagtctgaggacacagccatgtattactgtgcaaggggaagagtccgatcctactatggtaaccccgatgctatggacttctggggtcaaggaacctcagtcaccgtctcctca

SEQ ID NO: 96 - protein, 16 aa, Artificial Sequence
  Synthetic construct
RSSQSLVHSNGNTYLH

SEQ ID NO: 97 - protein, 7 aa, Artificial Sequence
  Synthetic construct
KVSKRFS

SEQ ID NO: 98 - protein, 9 aa, Artificial Sequence
  Synthetic construct
SQSIYVPHT

SEQ ID NO: 99 - protein, 112 aa, Artificial Sequence
  Synthetic construct
DVVMTQTPLSLPVSLGDQASISCRSSQSLVHSNGNTYLHWYLQKPGQSPKLLIYKVSKRFSGVPDRFSGSGSGTDFTLKITRVEAEDLGVYFCSQSIYVPHTFGGGTKLEIK

SEQ ID NO: 100 - protein, 131 aa, Artificial Sequence
  Synthetic construct
MKLPVRLLVLMFWIPASSSDVVMTQTPLSLPVSLGDQASISCRSSQSLVHSNGNTYLHWYLQKPGQSPKLLIYKVSKRFSGVPDRFSGSGSGTDFTLKITRVEAEDLGVYFCSQSIYVPHTFGGGTKLEIK

SEQ ID NO: 101 - DNA, 48 bp, Artificial Sequence
  Sythetic construct
agatctagtcagagccttgtacacagtaatggaaacacctatttacat

SEQ ID NO: 102 - DNA, 21 bp, Artificial Sequence
  Synthetic construct
aaagtttccaaacgattttct

SEQ ID NO: 103 - DNA, 27 bp, Artificial Sequence
  Synthetic construct
tctcaaagtatatatgttccgcacacg

SEQ ID NO: 104 - DNA, 336 bp, Artificial Sequence
  Synthetic construct
gatgttgtgatgacccaaactccactctccctgcctgtcagtcttggagatcaggcctccatctcttgcagatctagtcagagccttgtacacagtaatggaaacacctatttacattggtacctgcagaagccaggccagtctccaaagctcctgatctacaaagtttccaaacgattttctggggtcccagacaggttcagtggcagtggatcagggacagatttcacactcaagatcaccagagtggaggctgaggatctgggagtttatttctgctctcaaagtatatatgttccgcacacgttcggaggggggaccaagctggaaataaaa

SEQ ID NO: 105 - DNA, 393 bp, Artificial Sequence
  Synthetic construct
atgaagttgcctgttaggctgttggtgctgatgttctggattcctgcttccagcagtgatgttgtgatgacccaaactccactctccctgcctgtcagtcttggagatcaggcctccatctcttgcagatctagtcagagccttgtacacagtaatggaaacacctatttacattggtacctgcagaagccaggccagtctccaaagctcctgatctacaaagtttccaaacgattttctggggtcccagacaggttcagtggcagtggatcagggacagatttcacactcaagatcaccagagtggaggctgaggatctgggagtttatttctgctctcaaagtatatatgttccgcacacgttcggaggggggaccaagctggaaataaaa

SEQ ID NO: 106 - DNA, 15 bp, Artificial Sequence
  Synthetic construct
gactattacttatat

SEQ ID NO: 107 - DNA, 51 bp, Artificial Sequence; identical to SEQ ID NO: 92
  Synthetic construct

SEQ ID NO: 108 - DNA, 45 bp, Artificial Sequence; identical to SEQ ID NO: 93
  Synthetic construct

SEQ ID NO: 109 - DNA, 48 bp, Artificial Sequence; identical to SEQ ID NO: 101
  Synthetic construct

SEQ ID NO: 110 - DNA, 21 bp, Artificial Sequence
  Synthetic construct
aaagtttccaaacgattttct

SEQ ID NO: 111 - DNA, 27 bp, Artificial Sequence
  Synthetic construct
tctcaaagtatatatgttccgcacacg

SEQ ID NO: 112 - DNA, 20 bp, Artificial Sequence
  Synthetic construct
tagtagccgggccctacttt

(19) United States
(12) Patent Application Publication
(10) Pub. No.: US 2024/0287164 A1
(43) Pub. Date: Aug. 29, 2024

(54) ANTIBODIES SPECIFIC TO NELL2 AND METHODS OF USE

(71) Applicant: BOARD OF REGENTS, THE UNIVERSITY OF TEXAS SYSTEM, Austin, TX (US)

(72) Inventor: Yuzuru Shiio, San Antonio, TX (US)

(21) Appl. No.: 18/571,833

(22) PCT Filed: Jun. 28, 2022
(86) PCT No.: PCT/US2022/035239

§ 371 Date: Dec. 19, 2023

Related U.S. Application Data
(60) Provisional application No. 63/216,332, filed on Jun. 29, 2021.

(51) Int. Cl. C07K 16/18; A61K 39/00; A61P 35/00

(52) U.S. Cl. CPC C07K 16/18; A61P 35/00; A61K 2039/505; C07K 2317/73; C07K 2317/76

(57) ABSTRACT

Disclosed herein are anti-NELL2 antibodies and methods of treating Ewing sarcoma, neuroblastoma, and brain cancers in a subject.

### CROSS REFERENCE TO RELATED APPLICATIONS

This application claims the benefit of the filing date of U.S. Provisional Application No. 63/216,332, filed on Jun. 29, 2021. The content of this earlier filed application is hereby incorporated by reference in its entirety.

### REFERENCE TO A SEQUENCE LISTING

The Sequence Listing submitted herein as a text file named “21105_0083P1_SL.txt,” created on Jun. 16, 2022, and having a size of 40,960 bytes is hereby incorporated by reference pursuant to 37 C.F.R. § 1.52(e)(5).

### BACKGROUND

Ewing sarcoma affects males more often than females. It may affect individuals of any age, but most often occurs in individuals between 10 and 20 years of age. Ewing sarcoma is the second most common primary bone tumor in children and accounts for approximately 2% of childhood cancer diagnoses. The annual incidence of Ewing sarcoma is 2.93 children per 1,000,000. Approximately 200-250 children and adolescents in the United States are diagnosed with a tumor in the Ewing family of tumors each year. Two-thirds will be long-term survivors (more than five years). Multiple anticancer drugs (chemotherapy) in combination with surgical procedures and/or radiation are used to treat Ewing family of tumors and are associated with negative effects on the heart, lungs and bones. New treatments are needed.

### SUMMARY

Disclosed herein are isolated antibodies comprising a light chain variable region and a heavy chain variable region, wherein the light chain variable region comprises a complementarity determining region light chain 1 (CDRL1) amino acid sequence of SEQ ID NO: 1: a complementarity determining region light chain 2 (CDRL2) amino acid sequence of SEQ ID NO: 2; and a complementarity determining region light chain 3 (CDRL3) amino acid sequence of SEQ ID NO: 3; and wherein the heavy chain variable region comprises a complementarity determining region heavy chain 1 (CDRH1) amino acid sequence of SEQ ID NO: 11: a complementarity determining region heavy chain 2 (CDRH2) amino acid sequence of SEQ ID NO: 12; and a complementarity determining region heavy chain 3 (CDRH3) amino acid sequence of SEQ ID NO: 13.

Disclosed herein are isolated antibodies comprising a light chain variable region amino acid sequence of SEQ ID NO: 4 and a heavy chain variable region amino acid sequence of SEQ ID NO: 14.

Disclosed herein are isolated antibodies comprising a light chain variable region and a heavy chain variable region, wherein the light chain variable region comprises a complementarity determining region light chain 1 (CDRL1) amino acid sequence of SEQ ID NO: 1: a determining region light chain 2 (CDRL2) amino acid sequence of SEQ ID NO: 2; and a determining region light chain 3 (CDRL3) amino acid sequence of SEQ ID NO: 3; and wherein the heavy chain variable region comprises a complementarity determining region heavy chain 1 (CDRH1) amino acid sequence of SEQ ID NO: 11: a complementarity determining region heavy chain 2 (CDRH2) amino acid sequence of SEQ ID NO: 12; and a complementarity determining region heavy chain 3 (CDRH3) amino acid sequence of SEQ ID NO: 13, wherein one or more of the CDRL1, CDRL2, CDRL3, CDRH1, CDRH2, or CDRH3 comprise 1, 2, 3, 4, or 5 amino acid substitutions. In some aspects, the amino acid substitutions are conservative amino acid substitutions.

Disclosed herein are isolated antibodies comprising a light chain variable region amino acid sequence of SEQ ID NO: 4 and a heavy chain variable region amino acid sequence of SEQ ID NO: 14, wherein the isolated antibody comprises 1, 2, 3, 4, or 5 amino acid substitutions in the light or heavy chain variable region amino acid sequences. In some aspects, the amino acid substitutions are conservative amino acid substitutions.

Disclosed herein are isolated antibodies comprising a light chain variable region and a heavy chain variable region, wherein the light chain variable region comprises: a) a variant complementarity determining region light chain 1 (CDRL1) comprising positions 24-34 of SEQ ID NO: 4, wherein the variant CDRL1 comprises one or two amino acid substitutions; b) a variant complementarity determining region light chain 2 (CDRL2) comprising positions 50-56 of SEQ ID NO: 4, wherein the variant CDRL2 comprises one or two amino acid substitutions; and b) a variant complementarity determining region light chain 3 (CDRL3) comprising positions 89-97 of SEQ ID NO: 4, wherein the variant CDRL3 comprises one or two amino acid substitutions; wherein the heavy chain variable region comprises: d) a variant complementarity determining region heavy chain 1 (CDRH1) comprising positions 31-35 of SEQ ID NO: 14, wherein the variant CDRH1 comprises one or two amino acid substitutions; e) a variant complementarity determining region heavy chain 2 (CDRH2) comprising positions 50-66 of SEQ ID NO: 14, wherein the variant CDRH2 comprises one or two amino acid substitutions; and f) a variant complementarity determining region heavy chain 3 (CDRH3) comprising positions 99-111 of SEQ ID NO: 14, wherein the variant CDRH3 comprises one or two amino acid substitutions. In some aspects, the amino acid substitutions are conservative amino acid substitutions.

Disclosed are methods of treating a NELL2 positive cancer in a subject in need, the methods comprising administering to the subject having cancer a therapeutically effective amount of one or more of the isolated antibodies disclosed herein. Disclosed are methods of treating Ewing sarcoma, neuroblastoma or a brain cancer in a subject in need, the methods comprising administering to the subject having cancer a therapeutically effective amount of one or more of the isolated antibodies disclosed herein.

Disclosed are methods of blocking the binding of NELL2 with Robo3 in a subject, the methods comprising administering to the subject a therapeutically effective amount of one or more of the isolated antibodies disclosed herein. Disclosed are methods of increasing active cdc42 levels in a subject, the methods comprising administering to the subject a therapeutically effective amount of one or more of the isolated antibodies disclosed herein. Disclosed are methods of treating metastatic cancer in a subject or preventing metastasis in a subject at risk of metastasis, the methods comprising administering to the subject a therapeutically effective amount of one or more of the isolated antibodies disclosed herein.

### BRIEF DESCRIPTION OF THE DRAWINGS

FIGS. 1A-O show that Ewing sarcoma is dependent on NELL2, a target of EWS-FLI1. FIG. 1A shows an outline of secretome proteomic analysis. Note that EWS-FLI1 (FLI1 C-terminus) shRNA could also silence untranslocated FLI1, which is expressed at low levels. FIG. 1B shows shRNA-mediated silencing of EWS-FLI1 in A-673 cells. FIG. 1C shows that EWS-FLI1 silencing reduces NELL2 protein levels in A-673 secretome. The quantification based on spectral counting by mass spectrometry (left). The immunoblotting data (right). FIG. 1D shows that EWS-FLI1 silencing reduces NELL2 RNA levels in A-673 cells. *p<0.05 (n=3). FIG. 1E shows that EWS-FLI1 induces NELL2 RNA expression in human mesenchymal stem cells, putative cells of origin of Ewing sarcoma. The quantitative real-time RT-PCR data (left). *p<0.05 (n=3) The immunoblotting data (right). FIG. 1F shows that EWS-FLI1 binds to the NELL2 gene promoter. Chromatin-immunoprecipitation analysis for EWS-FLI1 binding to the promoter of NELL2 and known EWS-FLI1 target genes (NR0B1, GLI-1, and FOXO1) as well as control (GAPDH), with and without EWS-FLI1 silencing (n=3). FIG. 1G shows that NELL2 is highly expressed in Ewing sarcoma tumors and cell lines. NELL2 RNA expression was analyzed by qRT-PCR and was normalized to the levels in human mesenchymal stem cells (hMSCs) (n=3). FIG. 1H shows that NELL2 silencing inhibits Ewing sarcoma proliferation. NELL2 was silenced by siRNAs and cell proliferation was assessed by the IncuCyte live-cell imaging system. NELL2 silencing was verified by immunoblotting (bottom). FIG. 1I shows that recombinant NELL2 rescues proliferation arrest induced by NELL2 silencing. A-673 cells were transfected with NELL2 siRNAs and were treated with or without recombinant NELL2 (250) ng/ml). FIG. 1J shows that NELL2 siRNAs have little effect on the proliferation of 293 and HeLa cells. FIG. 1K shows that NELL2 RNA expression in A-673, 293, and HeLa cells, which was normalized to the levels in human mesenchymal stem cells (n=3). FIG. 1L shows that siRNA-mediated silencing of NELL2 RNA in 293 and HeLa cells (n=3). FIG. 1M shows that NELL2 silencing inhibits anchorage-independent growth of A-673 cells. shRNA-mediated silencing of NELL2 was verified by immunoblotting (right). *p<0.05 (6 independent experiments). FIG. 1N shows that NELL2 silencing inhibits xenograft tumorigenicity of A-673 cells. (n=5, p<0.05). FIG. 1O shows that Ewing sarcoma depends on autocrine signaling by NELL2, a novel EWS-FLI1 target.

FIGS. 2A-H shows that Robo3 serves as the NELL2 receptor in Ewing sarcoma. FIG. 2A shows Robo3 RNA expression in Ewing sarcoma tumors and cell lines (n=3). FIG. 2B shows NELL2 ligand-binding assays in A-673 cells. NELL2-FLAG bound to control siRNA transfected cells and the binding was abolished by Robo3 silencing. FIG. 2C shows NELL2 ligand-binding assays in COS cells. NELL2 fused to alkaline phosphatase (AP) or alkaline phosphatase alone was produced from transfected 293T cells and was incubated with COS cells that were transfected with vector, Robo3, or Robo1. The binding of NELL2-AP or AP to cells was visualized by alkaline phosphatase reaction. NELL2-AP specifically bound to Robo3-expressing COS cells. FIG. 2D shows that NELL2 silencing results in accumulation of lentivirally expressed Robo3-FLAG on A-673 cell surface, which was reversed by recombinant NELL2. The quantification of the fraction of cells with surface Robo3-FLAG staining, based on the counting of more than 200 cells, is shown on the right. *p<0.05 Scale bars: 10 μm. FIG. 2E shows that Robo3 silencing inhibits Ewing sarcoma proliferation. A-673, EW8, TC32, and TC71 cells were transfected with Robo3 siRNA pool or control siRNA pool and cell proliferation was assessed by IncuCyte (top). Robo3 silencing was verified by immunoblotting (bottom). FIG. 2F shows Robo3 siRNAs targeting different regions of Robo3 inhibit A-673 cell proliferation. A-673 cells were transfected with six Robo3 siRNAs that target different regions of Robo3. Cell proliferation was assessed by IncuCyte (top). Robo3 silencing was verified by immunoblotting (bottom). FIG. 2G shows that NELL2 needs Robo3 to simulate Ewing sarcoma proliferation. A-673 cells were transfected with NELL2 siRNAs, Robo3 siRNAs, and/or control siRNAs and were treated with or without recombinant NELL2 (250 ng/ml) as indicated. Cell proliferation was assessed by IncuCyte. FIG. 2H shows that Robo3 serves as the NELL2 receptor in Ewing sarcoma.

FIGS. 3A-K shows that NELL2 signaling downregulates cdc42 and upregulates the BAF complexes. FIG. 3A shows that silencing of srGAPs inhibits Ewing sarcoma proliferation. srGAP1 and srGAP2 were silenced by siRNAs and cell proliferation was assessed by IncuCyte (left). The silencing of srGAP1 and srGAP2 was verified by immunoblotting (right). FIG. 3B shows that NELL2 silencing or Robo3 silencing increases filopodia in Ewing sarcoma cells. The effect of NELL2 silencing or Robo3 silencing on actin cytoskeleton was assessed by phalloidin staining (red). Cell nuclei were stained by DAPI (blue). The quantification of filopodia using the filopodia image analysis software (Saha, T., et al. (2016). Molecular biology of the cell 27, 3616-3626) is shown at the bottom. For each sample, ten randomly chosen fields containing a total of 100-250 cells were analyzed. *p<0.05 compared with control siRNA transfected cells. Scale bars: 10 μm. FIG. 3C shows that NELL2 silencing activates cdc42 and Rac in Ewing sarcoma. A-673 and EW8 cells were transfected with NELL2 siRNAs or control siRNAs. Two days after transfection, the levels of GTP-bound, active cdc42, Rac, and Rho A were examined by GST-PAKI (for cdc42 and Rac1) or GST-Rhotekin-RBD (for Rho A) pull-down of whole cell lysate followed by anti-cdc42. Rac, and Rho A immunoblotting. FIG. 3D. shows that the regulation of cdc42 and Rac activities by NELL2 requires srGAPs. FIG. 3E shows that a selective cdc42 inhibitor, ML141, abrogates the proliferation inhibition by NELL2 silencing or Robo3 silencing in Ewing sarcoma. A-673 and EW8 cells were transfected with NELL2 siRNAs, Robo3 siRNAs, or control siRNAs and were treated with or without 5 μM ML141. Cell proliferation was assessed by IncuCyte. FIG. 3F shows that cdc42 silencing abrogates the proliferation inhibition by NELL2 silencing. The silencing of NELL2 and cdc42 was verified by immunoblotting (right). FIG. 3G shows that NELL2 signaling inhibits cdc42, the normal function of which is to promote filopodia formation and inhibit cell proliferation in Ewing sarcoma. FIG. 3H shows that NELL2 silencing reduces EWS-FLI1 target gene expression in Ewing sarcoma. A-673 and EW8 cells were transfected with NELL2 siRNAs or control siRNAs and the RNA expression of indicated genes was examined by qRT-PCR and is presented after normalization to the levels in control siRNAs transfected cells (blue). The expression of EWS-FLI1 target genes is reduced in NELL2-silenced cells (red). n=3 FIG. 3I shows that NELL2 silencing reduces the protein levels of some of the BAF subunits in Ewing sarcoma. A-673, EW8, TC32, TC71, and SK-N-MC cells were transfected with NELL2 siRNAs or control siRNAs and the levels of indicated BAF subunits were examined by immunoblotting. Tubulin serves as a loading control. NELL2 silencing reduced BRG1, BRM, BAF250A, BAF155, and BAF47. FIG. 3J shows that recombinant NELL2 restores the protein levels of BAF subunits in NELL2-silenced cells. A-673 and EW8 cells were transfected with NELL2 siRNAs or control siRNAs and were treated with recombinant NELL2 (250) ng/ml) for the indicated time (hours). The levels of BAF subunits were assessed by immunoblotting. Tubulin serves as a loading control. FIG. 3K shows that NELL2 signaling upregulates the BAF complexes and enhances the transcriptional output of EWS-FLI1.

FIGS. 4A-M shows that NELL2^{high}CD133^{high}EWS-FLI1^{high} and NELL2^{low}CD133^{low}EWS-FLI1^{low} populations in Ewing sarcoma display phenotypes consistent with high and low NELL2 signaling, respectively. FIG. 4A shows that NELL2 expression is heterogeneous in Ewing sarcoma tumor. NELL2 expression in surgically resected Ewing sarcoma tumor was examined by immunohistochemistry (brown signals). FIG. 4B shows that NELL2 expression is heterogeneous in Ewing sarcoma cells and correlates with EWS-FLI1 expression. NELL2 and EWS-FLI1 expression in A-673 cells was assessed by anti-NELL2 (red) and anti-FLI1 C-terminus (green) immunofluorescent staining. The nuclei were stained with DAPI. Scale bar: 10 μm. FIG. 4C shows that NELL2 expression correlates with CD133 expression in Ewing sarcoma cells. NELL2 and CD133 expression in A-673 cells was assessed by anti-NELL2 (green) and anti-CD133 (AC133, red) immunofluorescent staining. The nuclei were stained with DAPI. Scale bar: 10 μm. FIG. 4D shows that the CD133^{low} population displays lower NELL2, EWS-FLI1, BRG1, BAF250A, and BAF155 levels than the CD133^{high} population. A-673 cells were incubated with anti-CD133 (AC133) antibody and were sorted into the CD133^{high} and CD133^{low} populations. The expression of indicated proteins was assessed by immunoblotting. Tubulin serves as a loading control. FIG. 4E shows that the CD133^{low} population displays reduced EWS-FLI1 target gene expression. The RNA expression of indicated genes was assessed by qRT-PCR and is presented after normalization to the levels in the CD133^{high} population (blue). The expression of EWS-FLI1 target genes is reduced in the CD133^{low} population (red). n=3 FIG. 4F shows that the CD133^{low} population displays slower growth than the CD133^{high} population. FIG. 4G shows that the CD133^{low} population displays reduced NELL2 and EWS-FLI1 expression. A-673, CHLA-9, EW8, TC71, TC32, and SK-N-MC Ewing sarcoma cells were sorted into the CD133^{high} and the CD133^{low} populations and the RNA expression of CD133, NELL2, and EWS-FLI1 was assessed by qRT-PCR (n=3). FIG. 4H shows that the CD133^{low} population displays increased filopodia. Actin cytoskeleton was visualized by phalloidin staining (red). Cell nuclei were stained by DAPI (blue). Scale bars: 10 μm. FIG. 4I shows that NELL2 silencing inhibits the proliferation of both CD133^{high} and CD133^{low} populations. FIG. 4J shows that the CD133^{low} population displays reduced sphere formation. *p<0.05 (3 independent experiments). FIG. 4K shows that the CD133^{low} population displays reduced xenograft tumorigenicity. (n=5, p<0.05)

FIG. 4L shows that The CD133^{low} population displays reduced migration. *p<0.05 (3 independent experiments). FIG. 4M shows that prolonged cisplatin or doxorubicin treatment enriches the CD133^{low} population.

FIGS. 5A-M show that NELL2, CD133, and EWS-FLI1 positively regulate each other and increase the BAF subunits and cell proliferation in Ewing sarcoma. FIG. 5A shows that Slow growth of the CD133^{low} population can be rescued by recombinant NELL2. The CD133^{low} population was treated with the indicated concentration of recombinant NELL2, and cell proliferation was assessed by IncuCyte in comparison with the CD133^{high} population. FIG. 5B shows that recombinant NELL2 increases CD133 and BAF subunits in the CD133^{low} population. The CD133^{low} population was treated with the indicated concentration of recombinant NELL2 for four or twenty-four hours and the protein levels of CD133, BRG1, BAF250A, BAF155, and BAF47 were assessed by immunoblotting. Tubulin serves as a loading control. FIG. 5C shows NELL2 concentration in the culture supernatant of Ewing sarcoma cell lines. NELL2 concentration in the culture supernatant of five Ewing sarcoma cell lines, DMEM medium, and RPMI1640 medium was determined by ELISA (3 independent experiments). FIG. 5D shows that increasing CD133 in the CD133^{low} population results in increased NELL2, EWS-FLI1, BRG1, BAF250A, and BAF155. The CD133^{low} population was infected with CD133-expressing lentivirus, and the expression of indicated proteins was assessed by immunoblotting in comparison with uninfected CD133^{low} population and CD133^{high} population. Tubulin serves as a loading control. FIG. 5E shows that increasing CD133 in the CD133^{low} population results in increased cell proliferation. Proliferation of cells in FIG. 5D was assessed by IncuCyte. FIG. 5F shows that CD133 silencing results in reduced NELL2, EWS-FLI1, BRG1, and BAF155. A-673, EW8, TC71, and CHLA-9 cells were transfected with CD133 siRNAs (+) or control siRNAs (−), and the expression of indicated proteins was assessed by immunoblotting. Tubulin serves as a loading control. FIG. 5G shows that CD133 silencing results in reduced cell proliferation. A-673 cells were transfected with CD133 siRNAs or control siRNAs and cell proliferation was assessed by IncuCyte. FIG. 5H shows that NELL2 silencing results in reduced CD133. A-673 and EW8 cells were transfected with NELL2 siRNAs or control siRNAs, and the levels of NELL2 and CD133 were assessed by immunoblotting. Tubulin serves as a loading control. FIG. 5I shows that EWS-FLI1 silencing results in reduced NELL2 and CD133. A-673 and EW8 cells were infected with lentiviruses expressing FLI1 C-terminus shRNA (+) or control shRNA (−) and were selected with puromycin. The expression of EWS-FLI1, NELL2, and CD133 was assessed by immunoblotting. Tubulin serves as a loading control. FIG. 5J shows that EWS-FLI1 binds to the P2 and P6 promoters of the CD133 gene. Chromatin immunoprecipitation was performed as in FIG. 1F (n=3). FIG. 5K shows that EWS-FLI1 silencing results in reduced CD133 transcript levels in A-673 cells. *p<0.05 (n=3) FIG. 5L shows that EWS-FLI1 expression results in increased CD133 transcript levels in human mesenchymal stem cells. *p<0.05 (n=3) FIG. 5M shows that NELL2, CD133, and EWS-FLI1 positively regulate each other in Ewing sarcoma.

FIG. 6 shows that anti-NELL2 monoclonal antibodies inhibit Ewing sarcoma growth.

FIG. 7 shows that anti-NELL2 monoclonal antibodies inhibit xenograft tumor growth.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list. Further, the term “or” means “and/or” unless explicitly indicated to refer to alternatives only or the alternatives are mutually exclusive, although the disclosure supports a definition that refers to only alternatives and “and/or.”

As used herein, the term “another” means at least a second or more.

As used herein, the term “about” is used to indicate that a value includes the inherent variation of error for the device, the method being employed to determine the value, or the variation that exists among the study subjects.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In some aspects, a subject is a human. The term does not denote a particular age or sex. Thus, adult, child, adolescent and new born subjects, as well as fetuses, whether male or female, are intended to be covered. As used herein, the term “subject” refers to either a human or a non-human animal, such as primates, mammals, and vertebrates having cancer or diagnosed with cancer. In some aspects, the subject in need will or is predicted to benefit from anti-NELL2 antibody treatment.

As used herein, the term “patient” refers to a subject afflicted with a disease or disorder. The term “patient” includes human and veterinary subjects. In some aspects of the disclosed methods, the “patient” has been diagnosed with a need for treatment for cancer, such as, for example, prior to the administering step.

As used herein, the term “treat,” “treatment,” or “treating” refers to administration or application of a therapeutic agent to a subject in need thereof, or performance of a procedure or modality on a subject, for the purpose of obtaining at least one positive therapeutic effect or benefit, such as treating a disease or health-related condition. For example, a treatment can include administration of a pharmaceutically effective amount of an antibody, or a composition or formulation thereof that specifically binds to NELL2 for the purpose of treating cancer. The terms “treatment regimen,” “dosing regimen,” or “dosing protocol,” are used interchangeably and refer to the timing and dose of a therapeutic agent, such as an anti-NELL2 antibody as described herein.

As used herein, the term “therapeutic benefit” or “therapeutically effective” refers the promotion or enhancement of the well-being of a subject in need (e.g., a subject with cancer) with respect to the medical treatment, therapy, dosage administration, of a condition, particularly as a result of the use of the anti-NELL2 antibodies and the performance of the described methods. This includes, but is not limited to, a reduction in the frequency or severity of the signs or symptoms of a disease. In some aspects, treatment of cancer or metastatic cancer may involve, for instance, a reduction in the size of a tumor, a reduction in the invasiveness or severity of a tumor, a reduction infiltration of cancer cells into a peripheral tissue or organ: a reduction in the growth rate of the tumor or cancer, or the prevention or reduction of metastasis. Treatment of cancer may also refer to achieving a sustained response in a subject or prolonging the survival of a subject with cancer.

As used herein, the term “administer” or “administration” refers to the act of physically delivering, e.g., via injection or an oral route, a substance as it exists outside the body into a patient, such as by oral, subcutaneous, mucosal, intradermal, intravenous, intramuscular delivery and/or any other method of physical delivery described herein or known in the art. When a disease, disorder or condition, or a symptom thereof, is being treated therapeutically, administration of the substance typically occurs after the onset of the disease, disorder or condition or symptoms thereof. Prophylactic treatment involves the administration of the substance at a time prior to the onset of the disease, disorder or condition or symptoms thereof.

As used herein, the term “effective amount” refers to the quantity or amount of a therapeutic (e.g., an antibody or pharmaceutical composition provided herein) which is sufficient to reduce, diminish, alleviate, and/or ameliorate the severity and/or duration of a cancer or a symptom related thereto. This term also encompasses an amount necessary for the reduction or amelioration of the advancement or progression of a cancer: the reduction or amelioration of the recurrence, development of a cancer; and/or the improvement or enhancement of the prophylactic or therapeutic effect(s) of another cancer therapy. In some aspects, the effective amount of an antibody provided herein is from about or equal to 0.1 mg/kg (mg of antibody per kg weight of the subject) to about or equal to 100 mg/kg. In some aspects, an effective amount of an antibody provided therein is about or equal to 0.1 mg/kg, about or equal to 0.5 mg/kg, about or equal to 1 mg/kg, about or equal to 3 mg/kg, about or equal to 5 mg/kg, about or equal to 10 mg/kg, about or equal to 15 mg/kg, about or equal to 20 mg/kg, about or equal to 25 mg/kg, about or equal to 30 mg/kg, about or equal to 35 mg/kg, about or equal to 40 mg/kg, about or equal to 45 mg/kg, about or equal to 50 mg/kg, about or equal to 60 mg/kg, about or equal to 70 mg/kg, 80 mg/kg, 90 mg/kg, or 100 mg/kg. These amounts are meant to include amounts and ranges therein. In some aspects, “effective amount” also refers to the amount of an antibody provided herein to achieve a specified result (e.g., preventing, blocking, or inhibiting NELL2 binding to Robo3).

The term “in combination” in the context of the administration of other therapies (e.g., other agents, cancer drugs, cancer therapies) includes the use of more than one therapy (e.g., drug therapy and/or cancer therapy). Administration “in combination with” one or more further therapeutic agents includes simultaneous (e.g., concurrent) and consecutive administration in any order. The use of the term “in combination” does not restrict the order in which therapies are administered to a subject. By way of non-limiting example, a first therapy (e.g., agent, such as an anti-NELL2 antibody) may be administered before (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., agent) to a subject having or diagnosed with cancer.

The combination of therapies (e.g., use of agents, including therapeutic agents) may be more effective than the additive effects of any two or more single therapy (e.g., have a synergistic effect). A synergistic effect is typically unexpected and cannot be predicted. For example, a synergistic effect of a combination of therapeutic agents frequently permits the use of lower dosages of one or more of the agents and/or less frequent administration of the agents to a cancer patient. The ability to utilize lower dosages of therapeutics and cancer therapies and/or to administer the therapies less frequently reduces the potential for toxicity associated with the administration of the therapies to a subject without reducing the effectiveness of the therapies. In addition, a synergistic effect may result in improved efficacy of therapies in the treatment or alleviation of a cancer. Also, a synergistic effect demonstrated by a combination of therapies (e.g., therapeutic agents) may avoid or reduce adverse or unwanted side effects associated with the use of any single therapy.

As used herein, the term “comprising” can include the aspects “consisting of” and “consisting essentially of.” “Comprising” can also mean “including but not limited to.”

“Inhibit,” “inhibiting” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in some aspects, the increase or promotion can be a 10, 20, 30, 40, 50, 60, 70, 80, 90, 100%, or more, or any amount of promotion in between compared to native or control levels. In some aspects, the increase or promotion is 10-20, 20-30, 30-40, 40-50, 50-60, 60-70, 70-80, 80-90, or 90-100% as compared to native or control levels. In some aspects, the increase or promotion is 0-25, 25-50, 50-75, or 75-100%, or more, such as 200, 300, 500, or 1000% more as compared to native or control levels. In some aspects, the increase or promotion can be greater than 100 percent as compared to native or control levels, such as 100, 150, 200, 250, 300, 350, 400, 450, 500% or more as compared to the native or control levels.

As used herein, the term “determining” can refer to measuring or ascertaining a quantity or an amount or a change in activity. For example, determining the amount of a disclosed polypeptide, protein, gene or antibody in a sample as used herein can refer to the steps that the skilled person would take to measure or ascertain some quantifiable value of the polypeptide protein, gene or antibody in the sample. The art is familiar with the ways to measure an amount of the disclosed polypeptide, proteins, genes or antibodies in a sample.

As used herein, the terms “disease” or “disorder” or “condition” are used interchangeably referring to any alternation in state of the body or of some of the organs, interrupting or disturbing the performance of the functions and/or causing symptoms such as discomfort, dysfunction, distress, or even death to the person afflicted or those in contact with a person. A disease or disorder or condition can also related to a distemper, ailing, ailment, disorder, sickness, illness, complaint, affection. In some aspects, the disease or disorder or condition can be a cancer or metastatic cancer. In some aspects, the cancer can be a NELL2 positive cancer. In some aspects, the cancer can be a Ewing sarcoma, neuroblastoma or a brain cancer.

As used herein, the term “NELL2” refers to the protein kinase C-binding protein NELL2. NELL2 is an enzyme that in humans is encoded by the NELL2 gene. As used herein, the term “NELL2” refers to a polypeptide (the terms “polypeptide” and “protein” are used interchangeably herein) or any native NELL2 from any vertebrate source, including mammals such as primates (e.g., humans, cynomolgus monkey (cyno)), dogs, and rodents (e.g., mice and rats), unless otherwise indicated, and, in certain aspects, included various NELL2 isoforms, related NELL2 polypeptides, including SNP variants thereof. Also, as used herein, the terms “isolated antibody” or “isolated antibodies” can be used interchangeably with the terms “anti-NELL2 antibody” or “anti-NELL2 antibodies.”

An exemplary amino acid sequence of human NELL2 is accession number Q99435.

Abbreviations for the amino acid residues that comprise polypeptides and peptides described herein, and conservative substitutions for these amino acid residues are shown in Table 1 below: A polypeptide that contains one or more conservative amino acid substitutions or a conservatively modified variant of a polypeptide described herein refers to a polypeptide in which the original or naturally occurring amino acids are substituted with other amino acids having similar characteristics, for example, similar charge, hydrophobicity/hydrophilicity, side-chain size, backbone conformation, structure and rigidity, etc. Thus, these amino acid changes can typically be made without altering the biological activity, function, or other desired property of the polypeptide, such as its affinity or its specificity for antigen. In general, single amino acid substitutions in nonessential regions of a polypeptide do not substantially alter biological activity. Furthermore, substitutions of amino acids that are similar in structure or function are less likely to disrupt the polypeptides' biological activity.

TABLE 1
Amino Acid Residues and Examples of
Conservative Amino Acid Substitutions

| Original residue | |
|---|---|
| Three letter code and | Conservative |
| Single letter code | substitution(s) |
| Alanine (Ala) (A) | Gly; Ser |
| Arginine (Arg) (R) | Lys; His |
| Asparagine (Asn) (N) | Gln; His |
| Aspartic Acid (Asp) (D) | Glu; Asn |
| Cysteine (Cys) (C) | Ser; Ala |
| Glutamine (Gln) (Q) | Asn |
| Glutamic Acid (Glu) (E) | Asp; Gln |
| Glycine (Gly) (G) | Ala |
| Histidine (His) (H) | Asn; Gln |
| Isoleucine (Ile) (I) | Leu; Val |
| Leucine (Leu) (L) | Ile; Val |
| Lysine (Lys) (K) | Arg; His |
| Methionine (Met) (M) | Leu; Ile; Tyr |
| Phenylalanine (Phe) (F) | Tyr; Met; Leu |
| Proline (Pro) (P) | Ala |
| Serine (Ser) (S) | Thr |
| Threonine (Thr) (T) | Ser |
| Tryptophan (Trp) (W) | Tyr; Phe |
| Tyrosine (Tyr) (Y) | Trp; Phe |
| Valine (Val) (V) | Ile; Leu |

As used herein, the term “polypeptide” or “peptide” refers to a polymer of amino acids of three or more amino acids in a serial array, linked through peptide bonds. As used herein, the term “amino acid sequence” refers to a list of abbreviations, letters, characters or words representing amino acid residues. “Polypeptides” can be proteins, protein fragments, protein analogs, oligopeptides and the like. The amino acids that comprise the polypeptide may be naturally derived or synthetic. The polypeptide may be purified from a biological sample. For example, a NELL2 polypeptide or peptide may be composed of at least 3, 4, 5, 6, 7, 8, 9, 10, 11, 12, 13, 14, 15, 16, 17, 18, 19, 20, 21, 22, 23, 24, or 25 contiguous amino acids of human NELL2. In some aspects, the polypeptide has at least 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, or 816 contiguous amino acids of human NELL2. In some aspects, the NELL2 polypeptide comprises at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 150 contiguous amino acid residues, at least 175 contiguous amino acid residues, at least 200 contiguous amino acid residues, at least 225 contiguous amino acid residues, at least 250 contiguous amino acid residues, at least 275 contiguous amino acid residues, at least 300 contiguous amino acid residues, at least 325 contiguous amino acid residues, at least 350 contiguous amino acid residues, at least 375 contiguous amino acid residues, at least 400 contiguous amino acid residues, at least 425 contiguous amino acid residues, at least 450 contiguous amino acid residues, at least 475 contiguous amino acid residues, at least 500 contiguous amino acid residues, at least 525 contiguous amino acid residues, at least 550 contiguous amino acid residues, at least 575 contiguous amino acid residues, at least 600 contiguous amino acid residues, at least 625 contiguous amino acid residues, at least 650 contiguous amino acid residues, at least 675 contiguous amino acid residues, at least 700 contiguous amino acid residues, at least 725 contiguous amino acid residues, at least 750 contiguous amino acid residues, at least 775 contiguous amino acid residues, at least 800 contiguous amino acid residues, or at least 816 contiguous amino acid residues of the amino acid sequence of the NELL2 polypeptide.

By “isolated polypeptide” or “purified polypeptide” is meant a polypeptide (or a fragment thereof) that is substantially free from the materials with which the polypeptide is normally associated in nature. The polypeptides of the invention, or fragments thereof, can be obtained, for example, by extraction from a natural source (for example, a mammalian cell), by expression of a recombinant nucleic acid encoding the polypeptide (for example, in a cell or in a cell-free translation system), or by chemically synthesizing the polypeptide. In addition, polypeptide fragments may be obtained by any of these methods, or by cleaving full-length polypeptides.

As used herein, the term “analog” refers to a polypeptide that possesses a similar or identical function as a reference polypeptide but does not necessarily comprise a similar or identical amino acid sequence of the reference polypeptide, or possess a similar or identical structure of the reference polypeptide. The reference polypeptide may be a NELL2 polypeptide, a fragment of a NELL2 polypeptide, or an anti-NELL2 antibody. A polypeptide that has a similar amino acid sequence with a reference polypeptide refers to a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of the reference polypeptide, which can be a NELL2 polypeptide or an anti-NELL2 antibody as described herein. A polypeptide with similar structure to a reference polypeptide refers to a polypeptide that has a secondary, tertiary, or quaternary structure similar to that of the reference polypeptide, which can be a NELL2 polypeptide or an anti-NELL2 antibody described herein. The structure of a polypeptide can determined by methods known to those skilled in the art, including, but not limited to, X-ray crystallography, nuclear magnetic resonance (NMR), and crystallographic electron microscopy.

The term “fragment” can refer to a portion (e.g., at least 5, 10, 25, 50, 100, 125, 150, 200, 250, 300, 350, 400 or 500, etc. amino acids or nucleic acids) of a protein or nucleic acid molecule that is substantially identical to a reference protein or nucleic acid and retains the biological activity of the reference. In some aspects, the fragment or portion retains at least 50%, 75%, 80%, 85%, 90%, 95% or 99% of the biological activity of the reference protein or nucleic acid described herein. Further, a fragment of a referenced peptide can be a continuous or contiguous portion of the referenced polypeptide (e.g., a fragment of a peptide that is ten amino acids long can be any 2-9 contiguous residues within that peptide).

As used herein, the term “variant” when used in relation to a NELL2 polypeptide, an isolated antibody or to an anti-NELL2 antibody refers to a polypeptide, an isolated antibody or an anti-NELL2 antibody having one or more amino acid sequence substitutions, deletions, and/or additions as compared to a native or unmodified NELL2 sequence or anti-NELL2 antibody sequence. For example, a NELL2 polypeptide or to an anti-NELL2 antibody refers to a polypeptide or an anti-NELL2 antibody having one or more amino acid sequence substitutions, deletions, and/or additions as compared to a native or unmodified NELL2 sequence or anti-NELL2 antibody sequence can have about 1 to about 25, about 1 to about 20, about 1 to about 15, about 1 to about 10, or about 1 to about 5 amino acid sequence substitutions, deletions, and/or additions as compared to a native or unmodified NELL2 sequence or anti-NELL2 antibody sequence. A NELL2 variant can result from one or more (such as, for example, about 1 to about 25, about 1 to about 20, about 1 to about 15, about 1 to about 10, or about 1 to about 5) changes to an amino acid sequence of a native NELL2. Also by way of example, a variant of an anti-NELL2 antibody can result from one or more (such as, for example, about 1 to about 25, about 1 to about 20, about 1 to about 15, about 1 to about 10, or about 1 to about 5 changes to an amino acid sequence of a native or previously unmodified anti-NELL2 antibody. Variants can be naturally occurring, such as allelic or splice variants, or can be artificially constructed. Polypeptide variants can be prepared from the corresponding nucleic acid molecules encoding the variants.

A “variant” can mean a difference in some way from the reference sequence other than just a simple deletion of an N- and/or C-terminal amino acid residue or residues. Where the variant includes a substitution of an amino acid residue, the substitution can be considered conservative or non-conservative. Conservative substitutions can include those within the following groups: Ser, Thr, and Cys; Leu, Ile, and Val; Glu and Asp; Lys and Arg; Phe, Tyr, and Trp; and Gln, Asn, Glu, Asp, and His. Variants can include at least one substitution and/or at least one addition, there may also be at least one deletion. Variants can also include one or more non-naturally occurring residues. For example, a variant may include selenocysteine (e.g., seleno-L-cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH₂(CH₂)ₙCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties of proline.

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g., Met, Ala, Val, Leu, and Ile), among residues with neutral hydrophilic side chains (e.g., Cys, Ser, Thr, Asn and Gln), among residues with acidic side chains (e.g., Asp, Glu), among amino acids with basic side chains (e.g., His, Lys, and Arg), or among residues with aromatic side chains (e.g., Trp, Tyr, and Phe). As known in the art, conservative substitution usually does not cause significant change in the protein conformational structure, and therefore could retain the biological activity of a protein.

The term “identity.” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) must be addressed by a particular mathematical model or computer program (e.g., an “algorithm”). Methods that may be used to calculate the identity of the aligned nucleic acids or polypeptides include those described in Lesk, A. M., ed., 1988, Computational Molecular Biology, New York: Oxford University Press; Smith, D. W., ed., 1993, Biocomputing Informatics and Genome Projects, New York: Academic Press; Griffin, A. M., et al., 1994, Computer Analysis of Sequence Data, Part I, New Jersey: Humana Press; von Heinje, G., 1987, Sequence Analysis in Molecular Biology, New York: Academic Press; Gribskov, M. et al., 1991, Sequence Analysis Primer, New York: M. Stockton Press; and Carillo et al., 1988, Applied Math., 48:1073.

In calculating percent identity, the sequences being compared can be aligned in a way that gives the largest match between the sequences. An example of a computer program that can be used to determine percent identity is the GCG program package, which includes GAP (Devereux et al., 1984, Nucl. Acid Res., 12:387; Genetics Computer Group, University of Wisconsin, Madison, WI), which is a computer algorithm used to align the two polypeptides or polynucleotides to determine their percent sequence identity. The sequences can be aligned for optimal matching of their respective amino acid or nucleotide sequences (the “matched span” as determined by the algorithm). A gap opening penalty (which is calculated as 3 times the average diagonal, wherein the “average diagonal” is the average of the diagonal of the comparison matrix being used, and the “diagonal” is the score or number assigned to each perfect amino acid match by the particular comparison matrix; and a gap extension penalty (which is usually 1/10 times the gap opening penalty), as well as a comparison matrix such as PAM 250 or BLOSUM 62, are used in conjunction with the algorithm. In some aspects, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. USA 89:10915-10919 for the BLOSUM 62 comparison matrix) is also used by the algorithm. Exemplary parameters for determining percent identity for polypeptides or nucleotide sequences using the GAP program include the following: (i) Algorithm: Needleman et al., 1970, J. Mol. Biol., 48:443-453; (ii) Comparison matrix: BLOSUM 62 from Henikoff et al., Id.; (iii) Gap Penalty: 12 (but with no penalty for end gaps); (iv) Gap Length Penalty: 4; and (v) Threshold of Similarity: 0).

Certain alignment schemes for aligning two amino acid sequences can result in matching only a short region of the two sequences, and this small aligned region can have very high sequence identity even though there is no significant relationship between the two full-length sequences. Accordingly, the selected alignment method (e.g., the GAP program) can be adjusted if so desired to result in an alignment that spans a representative number of amino acids, for example, at least 50 contiguous amino acids, of the target polypeptide.

Percent (%) amino acid sequence identity with respect to a reference polypeptide sequence is defined as the percentage of amino acid residues in a candidate sequence that is identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill of the practitioner in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, the term “derivative” refers to a polypeptide that comprises an amino acid sequence of a reference polypeptide that has been altered by the introduction of amino acid residue substitutions, deletions or additions. The reference polypeptide can be a NELL2 polypeptide or an anti-NELL2 antibody. The term “derivative” as used herein also refers to a NELL2 polypeptide or an anti-NELL2 antibody that has been chemically modified, e.g., by the covalent attachment of any type of molecule to the polypeptide. For example, a NELL2 polypeptide or an anti-NELL2 antibody can be chemically modified, e.g., by glycosylation, acetylation, pegylation, phosphorylation, amidation, derivatization by known protecting/blocking groups, proteolytic cleavage, linkage to a cellular ligand, linkage to a peptide or protein tag molecule, or other protein, etc. The derivatives are modified in a manner that is different from the naturally occurring or starting peptide or polypeptides, either in the type or location of the molecules attached. Derivatives may further include deletion of one or more chemical groups which are naturally present on the peptide or polypeptide. A derivative of a NELL2 polypeptide or an anti-NELL2 antibody may be chemically modified by chemical modifications using techniques known to those of skill in the art, including, but not limited to, specific chemical cleavage, acetylation, formulation, metabolic synthesis by tunicamycin, etc. Further, a derivative of a NELL2 polypeptide or an anti-NELL2 antibody can contain one or more non-classical amino acids. A polypeptide derivative possesses a similar or identical function as the reference polypeptide, which can be a NELL2 polypeptide or an anti-NELL2 antibody described herein, especially an anti-NELL2 monoclonal antibody.

The term “fusion protein” as used herein refers to a polypeptide that includes amino acid sequences of at least two heterologous polypeptides. The term “fusion” when used in relation to a NELL2 polypeptide or to an anti-NELL2 antibody refers to the joining, fusing, or coupling of a NELL2 polypeptide or an anti-NELL2 antibody, variant and/or derivative thereof, with a heterologous peptide or polypeptide. In some aspects, the fusion protein retains the biological activity of the NELL2 polypeptide or the anti-NELL2 antibody. In some aspects, the fusion protein includes a NELL2 antibody VH region, VL region, VH CDR (one, two or three VH CDRs), and/or VL CDR (one, two or three VL CDRs) coupled, fused, or joined to a heterologous peptide or polypeptide, wherein the fusion protein binds to an epitope on a NELL2 protein or peptide. Fusion proteins may be prepared via chemical coupling reactions as practiced in the art, or via molecular recombinant technology.

As used herein, the term “composition” refers to a product containing specified component ingredients (e.g., a polypeptide or an antibody provided herein) in, optionally, specified or effective amounts, as well as any desired product which results, directly or indirectly, from the combination or interaction of the specific component ingredients in, optionally, the specified or effective amounts.

As used herein, the term “carrier” includes pharmaceutically acceptable carriers, excipients, diluents, vehicles, or stabilizers that are nontoxic to the cell or mammal being exposed thereto at the dosages and concentrations employed. Often, the physiologically acceptable carrier is an aqueous pH buffered solution. Examples of physiologically acceptable carriers include buffers such as phosphate, citrate, succinate, and other organic acids; antioxidants including ascorbic acid; low molecular weight (e.g., less than about 10) amino acid residues) polypeptides; proteins, such as serum albumin, gelatin, or immunoglobulins; hydrophilic polymers such as polyvinylpyrrolidone; amino acids such as glycine, glutamine, asparagine, histidine, arginine or lysine; monosaccharides, disaccharides, and other carbohydrates including glucose, mannose, sucrose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™. The term “carrier” can also refer to a diluent, adjuvant (e.g., Freund's adjuvant, complete or incomplete), excipient, or vehicle with which the therapeutic is administered. Such carriers, including pharmaceutical carriers, can be sterile liquids, such as water and oils, including those of petroleum, animal, vegetable or synthetic origin, such as peanut oil, soybean oil, mineral oil, sesame oil and the like. Water is an exemplary carrier when a composition (e.g., a pharmaceutical composition) is administered intravenously Saline solutions and aqueous dextrose and glycerol solutions can also be employed as liquid carriers, particularly for injectable solutions. Suitable excipients (e.g., pharmaceutical excipients) include, without limitation, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. Compositions can take the form of solutions, suspensions, emulsion, tablets, pills, capsules, powders, sustained-release formulations and the like. Oral compositions, including formulations, can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, (1990) Mack Publishing Co., Easton, PA. Compositions, including pharmaceutical compounds, can contain a therapeutically effective amount of an anti-NELL2 antibody in isolated or purified form, together with a suitable amount of carrier so as to provide the form for proper administration to the subject (e.g., patient). The composition or formulation should suit the mode of administration.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, PA, which is hereby incorporated by reference in its entirety.

As used herein, the term “pharmaceutically acceptable” or “pharmacologically acceptable” refers to molecular entities, formulations and compositions that do not produce an adverse, allergic, or other untoward or unwanted reaction when administered, as appropriate, to an animal, such as a human. The preparation of a pharmaceutical composition comprising an antibody or additional active ingredient are known to those of skill in the art in light of the present disclosure, as exemplified by Remington's Pharmaceutical Sciences, Id. Moreover, for animal (e.g., human) administration, it will be understood that preparations should meet sterility, pyrogenicity, general safety, and purity standards as required by a regulatory agency of the Federal or a state government, such as the FDA Office of Biological Standards or as listed in the U.S. Pharmacopeia, European Pharmacopeia, or other generally recognized Pharmacopeia for use in animals, and more particularly, in humans.

The term “pharmaceutical formulation” refers to a preparation which is in such form as to permit the biological activity of the active ingredient (e.g., an anti-NELL2 antibody) to be effective, and which contains no additional components that would be are unacceptably toxic to a subject to whom the formulation would be administered. Such a formulation can be sterile, i.e., aseptic or free from all living microorganisms and their spores, etc.

The term “package insert” is used to refer to instructions customarily included in commercial packages of therapeutic products, that contain information about the indications, usage, dosage, administration, contraindications and/or warnings concerning the use of such therapeutic products.

The terms “antibody,” “immunoglobulin,” and “Ig” are used interchangeably herein in a broad sense and specifically cover, for example, individual anti-NELL2 antibodies, such as the monoclonal antibodies described herein, (including agonist, antagonist, neutralizing antibodies, full length or intact monoclonal antibodies, peptide fragments of antibodies that maintain antigen binding activity); anti-NELL2 antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain anti-NELL2 antibodies, and fragments of anti-NELL2 antibodies, as described herein. An antibody can be human, humanized, chimeric and/or affinity matured. An antibody may be from other species, for example, mouse, rat, rabbit, etc. The term “antibody” is intended to include a polypeptide product of B cells within the immunoglobulin class of polypeptides that is able to bind to a specific molecular antigen. An antibody is typically composed of two identical pairs of polypeptide chains, wherein each pair has one heavy chain (about 50-70 kDa) and one light chain (about 25 kDa); and wherein the amino-terminal portion of the heavy and light chains includes a variable region of about 100 to about 130 or more amino acids and the carboxy-terminal portion of each chain includes a constant region (See, Borrebaeck (ed.), 1995, Antibody Engineering, Second Ed., Oxford University Press.; Kuby, 1997 Immunology, Third Ed., W.H. Freeman and Company, New York). In some aspects, the specific molecular antigen bound by an antibody provided herein includes a NELL2 polypeptide, a NELL2 peptide fragment, or a NELL2 epitope. An antibody or a peptide fragment thereof that binds to a NELL2 antigen can be identified, for example, by immunoassays, BIAcore, or other techniques known to those of skill in the art. An antibody or a fragment thereof binds specifically to a NELL2 antigen when it binds to a NELL2 antigen with higher affinity than to any cross-reactive antigen as determined using experimental techniques, such as radioimmunoassay's (RIA) and enzyme linked immunosorbent assays (ELISAs). Typically, a specific or selective binding reaction will be at least twice background signal or noise, and more typically more than 5-10 times background signal or noise. See, e.g., Paul, ed., 1989, Fundamental Immunology Second Edition, Raven Press, New York at pages 332-336 for a discussion regarding antibody specificity.

Antibodies provided herein include, but are not limited to, synthetic antibodies, monoclonal antibodies, recombinantly produced antibodies, multispecific antibodies (including bi-specific antibodies), human antibodies, humanized antibodies, camelized antibodies, chimeric antibodies, intrabodies, anti-idiotypic (anti-Id) antibodies, and functional fragments (e.g., antigen-binding fragments such as NELL2 binding fragments) of any of the above. A binding fragment refers to a portion of an antibody heavy or light chain polypeptide, such as a peptide portion, that retains some or all of the binding activity of the antibody from which the fragment is derived. Non-limiting examples of functional fragments (e.g., antigen-binding fragments such as NELL2 binding fragments) include single-chain Fvs (scFv) (e.g., including monospecific, bispecific, etc.), Fab fragments, F(ab) fragments, F(ab)₂ fragments, F(ab)₂ fragments, disulfide-linked Fvs (sdFv), Fd fragments, Fv fragments, diabodies, triabodies, tetrabodies and minibodies. In particular, antibodies provided herein include immunoglobulin molecules and immunologically active portions of immunoglobulin molecules, for example, antigen binding domains or molecules that contain an antigen-binding site that binds to a NELL2 antigen, (e.g., one or more complementarity determining regions (CDRs) of an anti-NELL2 antibody). Description of such antibody fragments can be found in, for example, Harlow and Lane, 1989, Antibodies: A Laboratory Manual, Cold Spring Harbor Laboratory, New York; Myers (ed.), Molec. Biology and Biotechnology: A Comprehensive Desk Reference, New York: VCH Publisher, Inc.; Huston et al., 1993, Cell Biophysics, 22: 189-224; Pluckthun and Skerra, 1989, Meth. Enzymol., 178:497-515 and in Day, E. D., 1990, Advanced Immunochemistry, Second Ed., Wiley-Liss, Inc., New York, NY. The antibodies provided herein can be of any type (e.g., IgG, IgE, IgM, IgD, IgA and IgY), any class (e.g., IgG1, IgG2, IgG3, IgG4, IgA1 and IgA2), or any subclass (e.g., IgG2a and IgG2b) of immunoglobulin molecule. Anti-NELL2 antibodies can be agonistic antibodies or antagonistic antibodies. In some aspects, the anti-NELL2 antibodies can be fully human, such as fully human monoclonal anti-NELL2 antibodies. In some aspects, the anti-NELL2 antibodies can be humanized, such as humanized monoclonal anti-NELL2 antibodies. In some aspects, the antibodies provided herein can be IgG antibodies, or a class (e.g., human IgG1 or IgG4) or subclass thereof, in particular, IgG1 subclass antibodies.

A four-chain antibody unit is a heterotetrameric glycoprotein composed of two identical light (L) chains and two identical heavy (H) chains. In the case of IgGs, the molecular weight of the four-chain (unreduced) antibody unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. At the N-terminus, each H chain has a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its carboxy terminus. The V_{L} domain is aligned with the V_{H} domain, and the C_{L} domain is aligned with the first constant domain of the heavy chain (C_{H1}). Particular amino acid residues are believed to form an interface between the light chain and heavy chain variable domains. The pairing of a V_{H} and V_{L} together forms a single antigen-binding site, although certain V_{H} and V_{L} domains can bind antigen without pairing with a V_{L} or V_{H} domain, respectively. The basic structure of immunoglobulin molecules is understood by those having skill in the art. For example, the structure and properties of the different classes of antibodies may be found in Terr, Abba I. et al., 1994, Basic and Clinical Immunology, 8th edition, Appleton & Lange, Norwalk, CT, page 71 and Chapter 6.

A “single-chain variable fragment (scFv)” means a protein comprising the variable regions of the heavy and light chains of an antibody. A scFv can be a fusion protein comprising a variable heavy chain, a linker, and a variable light chain. In some aspects, the linker can be a short, flexible fragment that can be about 8 to 20 amino acids in length. For example, (G4S)n can be used (n=1, 2, 3 or 4).

A “fragment antigen-binding fragment (Fab)” is a region of an antibody that binds to antigen. An Fab comprises constant and variable regions from both heavy and light chains.

A “CDR” or complementarity determining region is a region of hypervariability interspersed within regions that are more conserved, termed “framework regions” (FR).

As used herein, the term “antigen” or “target antigen” is a predetermined molecule to which an antibody can selectively bind. A target antigen can be a polypeptide, peptide, carbohydrate, nucleic acid, lipid, hapten, or other naturally occurring or synthetic compound. In some aspects, a target antigen can be a small molecule. In some aspects, the target antigen can a polypeptide or peptide, e.g., NELL2.

As used herein, the term “antigen binding fragment.” “antigen binding domain,” “antigen binding region,” and similar terms refer to that portion of an antibody which includes the amino acid residues that interact with an antigen and confer on the antibody as binding agent its specificity and affinity for the antigen (e.g., the CDRs of an antibody are antigen binding regions). The antigen binding region can be derived from any animal species, such as rodents (e.g., rabbit, rat, or hamster) and humans. In some aspects, the antigen binding region can be of human origin.

An “isolated” antibody is substantially free of cellular material or other contaminating proteins from the cell or tissue source and/or other contaminant components from which the antibody is derived, or is substantially free of chemical precursors or other chemicals when chemically synthesized. The language “substantially free of cellular material” includes preparations of an antibody in which the antibody is separated from cellular components of the cells from which it is isolated or recombinantly produced. Thus, an antibody that is substantially free of cellular material includes preparations of an antibody that have less than about 30%, 25%, 20%, 15%, 10%, 5%, or 1% (by dry weight) of heterologous protein (also referred to herein as a “contaminating protein”). In some aspects, when the antibody is recombinantly produced, it is substantially free of culture medium, e.g., culture medium represents less than about 20%, 15%, 10%, 5%, or 1% of the volume of the protein preparation. In some aspects, when the antibody is produced by chemical synthesis, it is substantially free of chemical precursors or other chemicals, for example, it is separated from chemical precursors or other chemicals which are involved in the synthesis of the protein. Accordingly such preparations of the antibody have less than about 30%, 25%, 20%, 15%. 10%, 5%, or 1% (by dry weight) of chemical precursors or compounds other than the antibody of interest. Contaminant components can also include, but are not limited to, materials that would interfere with therapeutic uses for the antibody, and can include enzymes, hormones, and other proteinaceous or nonproteinaceous solutes. In some aspects, the antibody is purified (1) to greater than or equal to 95% by weight of the antibody, as determined by the Lowry method (Lowry et al., 1951, J. Bio. Chem., 193: 265-275), such as 95%, 96%, 97%, 98%, or 99%, by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or silver stain. Isolated antibody also includes the antibody in situ within recombinant cells since at least one component of the antibody's natural environment will not be present. An isolated antibody is typically prepared by at least one purification step. In some aspects, the antibodies provided herein are isolated.

The term “monoclonal antibody.” (monoclonal antibody) refers to an antibody, or population of like antibodies, obtained from a population of substantially homogeneous antibodies, and is not to be construed as requiring production of the antibody by any particular method, including but not limited to, monoclonal antibodies can be made by the hybridoma method first described by Kohler and Milstein (Nature, 256: 495-497, 1975), or by recombinant DNA methods.

As used herein, the term “binds” or “binding” refers to an interaction between molecules including, for example, to form a complex. Illustratively, such interactions embrace non-covalent interactions, including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site of an antibody and its epitope on a target (antigen) molecule, such as NELL2, is the affinity of the antibody or functional fragment for that epitope. The ratio of association (kₒₙ) to dissociation (k_{off}) of an antibody to a monovalent antigen (kₒₙ/k_{off}) is the association constant Kₐ, which is a measure of affinity. The value of K varies for different complexes of antibody and antigen and depends on both kₒₙ and k_{off}. The association constant Kₐ for an antibody provided herein may be determined using any method provided herein or any other method known to those skilled in the art. The affinity at one binding site does not always reflect the true strength of the interaction between an antibody and an antigen. When complex antigens containing multiple, repeating antigenic determinants come into contact with antibodies containing multiple binding sites, the interaction of antibody with antigen at one site will increase the probability of an interaction at a second binding site. The strength of such multiple interactions between a multivalent antibody and antigen is called the avidity. The avidity of an antibody can be a better measure of its binding capacity than is the affinity of its individual binding sites. For example, high avidity can compensate for low affinity as is sometimes found for pentameric IgM antibodies, which can have a lower affinity than IgG, but the high avidity of IgM, resulting from its multivalence, enables it to bind antigen effectively.

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{d}). Affinity can be measured by common methods known in the art, including those described herein. Low-affinity antibodies generally bind antigen slowly and tend to dissociate readily, while high-affinity antibodies generally bind antigen faster and tend to remain bound longer to antigen. A variety of methods for measuring binding affinity are known in the art, any of which may be used for purposes of the present disclosure. Specific illustrative aspects include the following: In some aspects, the “K_{d}” or “K_{d} value” is measured by assays known in the art, for example, by a binding assay. The K_{d} can be measured in a radiolabeled antigen binding assay (RIA), for example, performed with the Fab portion of an antibody of interest and its antigen (Chen, et al., 1999, J. Mol. Biol., 293:865-881). The K_{d} or K_{d} value may also be measured by using surface plasmon resonance (SPR) assay's (by BIAcore) using, for example, a BIAcore™-2000 or a BIAcore™-3000 (BIAcore, Inc., Piscataway, NJ), or by biolayer interferometry (BLI) using, for example, the OctetQK384 system (ForteBio, Menlo Park, CA), or by quartz crystal microbalance (QCM) technology. An “on-rate” or “rate of association” or “association rate” or “kₒₙ” can also be determined with the same surface plasmon resonance or biolayer interferometry techniques described above, using, for example, a BIAcore™-2000 or a BIAcore™-3000 (BIAcore, Inc., Piscataway, NJ), or the OctetQK384 system (ForteBio, Menlo Park, CA).

Disclosed herein are anti-NELL2 antibodies, antibodies that specifically bind to NELL2, antibodies that are specific for NELL2, antibodies that specifically bind to a NELL2 epitope, antibodies that selectively bind to a NELL2 epitope, and antibodies that preferentially binds to NELL2. The terms “anti-NELL2 antibody,” “an antibody that specifically binds to NELL2,” or “antibody that is specific for NELL2,” “antibodies that specifically bind to a NELL2 epitope,” “an antibody that selectively binds to NELL2,” “antibodies that selectively bind to a NELL2 epitope,” “an antibody that preferentially binds to NELL2”, and analogous terms refer to antibodies capable of binding NELL2, i.e., wild-type (WT) NELL2, with sufficient affinity and specificity, particularly compared with mutants of NELL2.

By “specifically binds” is meant that an antibody recognizes and physically interacts with its cognate antigen (for example, NELL2) and does not significantly recognize and interact with other antigens; such an antibody may be a polyclonal antibody or a monoclonal antibody, which are generated by techniques that are well known in the art.

“Preferential binding” of the anti-NELL2 antibodies as provided herein may be determined or defined based on the quantification of fluorescence intensity of the antibodies binding to NELL2, i.e., NELL2 polypeptide, or WT NELL2, or NELL2 expressed on cells versus an appropriate control, such as binding to variant NELL2, or to cells expressing a variant form of NELL2, for example, molecularly engineered cells, cell lines or tumor cell isolates. Preferential binding of an anti-NELL2 antibody as described to a NELL2 WT-expressing cell is indicated by a measured fluorescent binding intensity (MFI) value, as assessed by cell flow cytometry, of at least 2-fold, at least 3-fold, at least 4-fold, at least 5-fold, at least 6-fold, at least 7-fold, at least 8-fold, at least 10-fold, at least 15-fold, at least 20-fold or greater, as compared with binding of the antibody to a mutant NELL2 polypeptide or a mutant NELL2-expressing cell, wherein the antibody to be assayed is directly or indirectly detectable by a fluorescent label or marker, such as FITC. In some aspects, the antibody to be assayed is directly labeled with a fluorescent marker, such as FITC. In some aspects, an anti-NELL2 antibody that preferentially or selectively binds NELL2 exhibits an MFI value of from 1.5-fold to 25-fold, or from 2-fold to 20-fold, or from 3-fold to 15-fold, or from 4-fold to 8-fold, or from 2-fold to 10-fold, or from 2-fold to 5-fold or more greater than the MFI value of the same antibody for binding a NELL2 or a NELL2 variant. Fold-fluorescence intensity values between and equal to all of the foregoing are intended to be included. In some aspects, the anti-NELL2 antibodies specifically and preferentially bind to a NELL2 polypeptide, such as a NELL2 antigen, peptide fragment, or epitope (e.g., human NELL2 such as a human NELL2 polypeptide, antigen or epitope). An antibody that specifically binds to NELL2, (e.g., wild type human NELL2) can bind to the extracellular domain (ECD) or a peptide derived from the ECD of NELL2. An antibody that specifically binds to a NELL2 antigen (e.g., human NELL2) can be cross-reactive with related antigens (e.g., cynomolgus (cyno) NELL2). In some aspects, an antibody that specifically binds to a NELL2 antigen does not cross-react with other antigens. An antibody that specifically binds to a NELL2 antigen can be identified, for example, by immunofluorescence binding assays, immunohistochemistry assay methods, immunoassay methods, Biacore, or other techniques known to those of skill in the art.

In some aspects, an antibody that binds to NELL2, as described herein, has a dissociation constant (K_{d}) of less than or equal to 100 nM, 95 nM, 90 nM, 85 nM, 80 nM, 75 nM, 70 nM, 65 nM, 60 nM, 55 nM, 50 nM, 45 nM, 40 nM, 35 nM, 30 nM, 25 nM, 20 nM, 15 nM, 10 nM, 5 nM, 4 nM, 3 nM, 2 nM, 1 nM, 05 nM, or 0.1 nM, and/or is greater than or equal to 0.1 nM. In some aspects, an anti-NELL2 antibody binds to an epitope of NELL2 that is conserved among NELL2 proteins from different species (e.g., between human and mouse NELL2). An antibody binds specifically to a NELL2 antigen when it binds to a NELL2 antigen with higher affinity than to any cross reactive antigen as determined using experimental techniques, such as radioimmunoassay's (RIA) and enzyme linked immunosorbent assays (ELISAs). Typically a specific or selective reaction will be at least twice background signal or noise and can be more than 10 times background. See, e.g., Paul, ed., 1989, Fundamental Immunology Second Edition, Raven Press, New York at pages 332-336 for a discussion regarding antibody specificity. In some aspects, the extent of binding of the antibody to a “non-target” protein will be less than about 10% of the binding of the antibody to its particular target protein, for example, as determined by fluorescence activated cell sorting (FACS) analysis or radioimmunoprecipitation (RIA).

As used herein, in reference to an antibody, the term “heavy (H) chain” refers to a polypeptide chain of about 50-70 kDa, wherein the amino-terminal portion includes a variable (V) region (also called V domain) of about 115 to 130 or more amino acids and a carboxy-terminal portion that includes a constant (C) region. The constant region (or constant domain) can be one of five distinct types, (e.g., isotypes) referred to as alpha (α), delta (δ), epsilon (ε), gamma (γ) and mu (μ), based on the amino acid sequence of the heavy chain constant region. The distinct heavy chains differ in size: α, δ and γ contain approximately 450 amino acids, while u and & contain approximately 550 amino acids. When combined with a light chain, these distinct types of heavy chains give rise to five well known classes (e.g., isotypes) of antibodies, namely, IgA, IgD, IgE, IgG and IgM, respectively, including four subclasses of IgG, namely IgG1, IgG2, IgG3 and IgG4. An antibody heavy chain can be a human antibody heavy chain.

As used herein in reference to an antibody, the term “light (L) chain” refers to a polypeptide chain of about 25 kDa, wherein the amino-terminal portion includes a variable domain of about 100 to about 110 or more amino acids and a carboxy-terminal portion that includes a constant region. The approximate length of a light chain (both the V and C domains) is 211 to 217 amino acids. There are two distinct types of light chains, referred to as kappa (κ) and lambda (λ.), based on the amino acid sequence of the constant domains. Light chain amino acid sequences are well known in the art. An antibody light chain can be a human antibody light chain.

As used herein, the term “variable (V) region” or “variable (V) domain” refers to a portion of the light (L) or heavy (H) chains of an antibody polypeptide that is generally located at the amino-terminus of the L or H chain. The H chain V domain has a length of about 115 to 130 amino acids, while the L chain V domain is about 100 to 110 amino acids in length. The H and L chain V domains are used in the binding and specificity of each particular antibody for its particular antigen. The V domain of the H chain can be referred to as “V_{H}.” The V region of the L chain can be referred to as “V_{L}.” The term “variable” refers to the fact that certain segments of the V domains differ extensively in sequence among different antibodies. While the V domain mediates antigen binding and defines specificity of a particular antibody for its particular antigen, the variability is not evenly distributed across the 110-amino acid span of antibody V domains. Instead, the V domains consist of less variable (e.g., relatively invariant) stretches called framework regions (FRs) of about 15-30 amino acids separated by shorter regions of greater variability (e.g., extreme variability) called “hypervariable regions” or “complementarity determining regions” (CDRs) that are each about 9-12 amino acids long or 3-17 amino acids long. The V domains of antibody H and L chains each comprise four FRs, largely adopting a β sheet configuration, connected by three hypervariable regions, called, which form loops connecting, and in some cases forming part of, the β sheet structure. The hypervariable regions in each chain are held together in close proximity by the FRs and, with the hypervariable regions from the other chain, contribute to the formation of the antigen-binding site of antibodies (see, e.g., Kabat et al., 1991, Sequences of Proteins of Immunological Interest, 5th Ed. Public Health Service, National Institutes of Health, Bethesda, MD). The C domains are not involved directly in binding an antibody to an antigen, but exhibit various effector functions, such as antibody dependent cellular cytotoxicity (ADCC) and complement dependent cytotoxicity (CDC). The V domains differ extensively in sequence among different antibody classes or types. The variability in sequence is concentrated in the CDRs, which are primarily responsible for the interaction of the antibody with antigen. In some aspects, the variable domain of an antibody is a human or humanized variable domain.

As used herein, the terms “complementarity determining region,” “CDR.” “hypervariable region,” “HVR,” and “HV” are used interchangeably. A “CDR” refers to one of three hypervariable regions (H1, H2 or H3) within the non-framework region of the antibody V_{H} β-sheet framework, or to one of three hypervariable regions (L1, L2 or L3) within the non-framework region of the antibody V_{L} β-sheet framework. The term, when used herein, refers to the regions of an antibody V domain that are hypervariable in sequence and/or form structurally defined loops. Generally, antibodies comprise six hypervariable regions: three (H1, H2, H3) in the V_{H} domain and three (L1, L2, L3) in the V_{L} domain. Accordingly, CDRs are typically highly variable sequences interspersed within the framework region sequences of the V domain. “Framework” or “FR” residues are those variable region residues flanking the CDRs. FR residues are present, for example, in chimeric, humanized, human, domain antibodies, diabodies, linear antibodies, and bispecific antibodies.

A number of hypervariable region delineations are in use and are encompassed herein. CDR regions are well known to those skilled in the art and have been defined by, for example, Kabat as the regions of most hypervariability within the antibody V domains (Kabat et al., 1977, J. Biol. Chem., 252:6609-6616; Kabat, 1978, Adv. Prot. Chem., 32: 1-75). The Kabat CDRs are based on sequence variability and are the most commonly used (see, e.g., Kabat et al., 1991, Sequences of Proteins of Immunological Interest, 5th Ed. Public Health Service, National Institutes of Health, Bethesda, MD). CDR region sequences also have been defined structurally by Chothia as those residues that are not part of the conserved ß-sheet framework, and thus are able to adopt different conformations (Chothia et al., 1987, J. Mol. Biol., 196:901-917). Chothia refers instead to the location of the structural loops. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). Both numbering systems and terminologies are well recognized in the art.

Recently, a universal numbering system has been developed and widely adopted, ImMunoGeneTics (IMGT) Information System® (Lafranc et al., 2003, Dev. Comp. Immunol., 27(1):55-77). IMGT is an integrated information system specializing in immunoglobulins (Ig), T cell receptors (TR) and the major histocompatibility complex (MHC) of human and other vertebrates. Herein, the CDRs are referred to in terms of both the amino acid sequence and the location within the light or heavy chain. As the “location” of the CDRs within the structure of the immunoglobulin V domain is conserved between species and present in structures called loops, by using numbering systems that align variable domain sequences according to structural features, CDR and framework residues and are readily identified. This information can be used in grafting and in the replacement of CDR residues from immunoglobulins of one species into an acceptor framework from, typically, a human antibody. An additional numbering system (AHon) has been developed by Honegger et al., 2001, J. Mol. Biol., 309: 657-670. Correspondence between the numbering system, including, for example, the Kabat numbering and the IMGT unique numbering system, is well known to one skilled in the art (see, e.g., Kabat, Id: Chothia et al., Id.; Martin, 2010, Antibody Engineering, Vol. 2, Chapter 3, Springer Verlag; and Lefranc et al., 1999, Nuc. Acids Res., 27:209-212).

CDR region sequences have also been defined by AbM, Contact and IMGT. The AbM hypervariable regions represent a compromise between the Kabat CDRs and Chothia structural loops, and are used by Oxford Molecular's AbM antibody modeling software (see, e.g., Martin, 2010, Antibody Engineering, Vol. 2, Chapter 3, Springer Verlag). The “contact” hypervariable regions are based on an analysis of the available complex crystal structures. The residues from each of these hypervariable regions or CDRs are noted below.

Exemplary delineations of CDR region sequences are illustrated in Table 2. The positions of CDRs within a canonical antibody variable region have been determined by comparison of numerous structures (Al-Lazikani et al., 1997, J. Mol. Biol., 273:927-948); Morea et al., 2000, Methods, 20:267-279). Because the number of residues within a hypervariable region varies in different antibodies, additional residues relative to the canonical positions are conventionally numbered with a, b, c and so forth next to the residue number in the canonical variable region numbering scheme (Al-Lazikani et al., Id). Such nomenclature is similarly well known to those skilled in the art. In some aspects, the exemplary delineations of CDR region sequences can be found here: bioinf.org.uk/abs/info.html#kabatnum.

TABLE 2
Exemplary Delineations of CDR Region Sequences

| | IMGT | Kabat |
|---|---|---|
| V_{H} CDR1 | 28-35 | 31-35 |
| V_{H} CDR2 | 51-58 | 47-56 |
| V_{H} CDR3 | 97-103 | 99-103 |
| V_{L} CDR1 | 27-32 | 24-34 |
| V_{L} CDR2 | 50-52 | 50-56 |
| V_{L} CDR3 | 89-97 | 89-97 |

An “affinity matured” antibody is one with one or more alterations (e.g., amino acid sequence variations, including changes, additions and/or deletions) in one or more HVRs thereof that result in an improvement in the affinity of the antibody for antigen, compared to a parent antibody which does not possess those alteration(s). In some aspects, affinity matured antibodies will have nanomolar or even picomolar affinities for the target antigen, such as NELL2. Affinity matured antibodies are produced by procedures known in the art. For reviews, see Hudson and Souriau, 2003, Nature Medicine, 9:129-134; Hoogenboom, 2005, Nature Biotechnol., 23:1105-1116; Quiroz and Sinclair, 2010, Revista Ingeneria Biomedia, 4: 39-51.

A “chimeric” antibody is one in which a portion of the H and/or L chain, e.g., the V domain, is identical with or homologous to a corresponding amino acid sequence in an antibody derived from a particular species or belonging to a particular antibody class or subclass, while the remainder of the chain(s), e.g., the C domain, is identical with or homologous to corresponding sequences in antibodies derived from another species or belonging to another antibody class or subclass, as well as a fragment of such an antibody, so long as it exhibits the desired biological activity (see, e.g., U.S. Pat. No. 4,816,567; and Morrison et al., 1984, Proc. Natl. Acad. Sci. USA, 81:6851-6855).

The term “humanized antibody” refers to forms of antibodies that contain sequences from non-human (e.g., murine) antibodies as well as human antibodies. A humanized antibody can include conservative amino acid substitutions or non-natural residues from the same or different species that do not significantly alter its binding and/or biologic activity. Such antibodies are chimeric antibodies that contain minimal sequence derived from non-human immunoglobulins. For the most part, humanized antibodies are human immunoglobulins (recipient antibody) in which residues from a complementary-determining region (CDR) of the recipient are replaced by residues from a CDR of a non-human species (donor antibody) such as mouse, rat, camel, bovine, goat, or rabbit having the desired properties. Furthermore, humanized antibodies can comprise residues that are found neither in the recipient antibody nor in the imported CDR or framework sequences. These modifications are made to further refine and maximize antibody performance. Thus, in general, a humanized antibody can comprise all or substantially all of at least one, and in one aspect two, variable domains, in which all or substantially all of the hypervariable loops correspond to those of a non-human immunoglobulin and all or substantially all of the FR regions are those of a human immunoglobulin sequence. The humanized antibody optionally also can comprise at least a portion of an immunoglobulin constant region (Fc), or that of a human immunoglobulin (see, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European U.S. Pat. No. 239,400 B1; Padlan, E. A. et al., European Patent Application No. 0,519,596 A1: Queen et al. (1989) Proc. Natl. Acad. Sci. USA, Vol 86:10029-10033). The terms “human antibody” and “fully human antibody” are used interchangeably herein and refer to an antibody that possesses an amino acid sequence which corresponds to that of an antibody produced by a human and/or has been made using any of the techniques for making human antibodies as practiced by those skilled in the art. This definition of a human antibody specifically excludes a humanized antibody comprising non-human antigen-binding residues. Human antibodies can be produced using various techniques known in the art, including phage-display libraries (Hoogenboom et al., 1991, J. Mol. Biol., 227:381; Marks et al., 1991, J. Mol. Biol., 222:581 and yeast display libraries (Chao et al., 2006, Nature Protocols, 1:755-768). Also available for the preparation of human monoclonal antibodies are methods described in Cole et al., 1985 Monoclonal Antibodies and Cancer Therapy, Alan R. Liss, p. 77: Boerner et al., 1991, J. Immunol., 147(1): 86-95. See also van Dijk et al., 2001, Curr. Opin. Pharmacol., 5: 368-74. Human antibodies can be prepared by administering an antigen to a transgenic animal whose endogenous Ig loci have been disabled, e.g., a mouse, and that has been genetically modified to harbor human immunoglobulin genes which encode human antibodies, such that human antibodies are generated in response to antigenic challenge (see, e.g., Jakobovits, A., 1995, Curr. Opin. Biotechnol. 6(5):561-566; Brüggemann et al., 1997 Curr. Opin. Biotechnol., 8(4):455-8; and U.S. Pat. Nos. 6,075,181 and 6,150,584 regarding XENOMOUSE™ technology). See also, for example, Li et al., 2006, Proc. Natl. Acad. Sci. USA, 103:3557-3562 regarding human antibodies generated via a human B-cell hybridoma technology. In some aspects, human antibodies comprise a variable region and constant region of human origin. “Fully human” anti-NELL2 antibodies, in some aspects, can also encompass antibodies which bind NELL2 polypeptides and are encoded by nucleic acid sequences which are naturally occurring somatic variants of human germline immunoglobulin nucleic acid sequence. In some aspects, the anti-NELL2 antibodies provided herein are fully human antibodies. The term “fully human antibody” includes antibodies having variable and constant regions corresponding to human germline immunoglobulin sequences as described by Kabat et al. (See Kabat et al., 1991, Sequences of Proteins of Immunological Interest, Fifth Edition, U.S. Department of Health and Human Services, NIH Publication No. 91-3242). The phrase “recombinant human antibody.” includes human antibodies that are prepared, expressed, created, or isolated by recombinant means, such as antibodies expressed using a recombinant expression vector transfected into a host cell; antibodies isolated from a recombinant, combinatorial human antibody library; antibodies isolated from an animal (e.g., a mouse or cow) that is transgenic and/or transchromosomal for human immunoglobulin genes (see e.g., Taylor, L. D. et al., 1992, Nucl. Acids Res. 20:6287-6295); or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat et al., 1991, Sequences of Proteins of Immunological Interest, Fifth Edition, U.S. Department of Health and Human Services, NIH Publication No. 91-3242). In some aspects, however, such recombinant human antibodies are subjected to in vitro mutagenesis (or, when an animal transgenic for human Ig sequences is used, in vivo somatic mutagenesis) and, thus, the amino acid sequences of the VH and VL regions of the recombinant antibodies are sequences that, while derived from and related to human germline VH and VL sequences, may not naturally exist within the human antibody germline repertoire in vivo.

As used herein, the term “epitope” is the site(s) or region(s) on the surface of an antigen molecule to which a single antibody molecule binds, such as a localized region on the surface of an antigen, e.g., a NELL2 polypeptide that is capable of being bound by one or more antigen binding regions of an anti-NELL2 antibody. An epitope can be immunogenic and capable of eliciting an immune response in an animal. Epitopes need not necessarily be immunogenic. Epitopes often consist of chemically active surface groupings of molecules such as amino acids or sugar side chains and have specific three dimensional structural characteristics as well as specific charge characteristics. An epitope can be a linear epitope and a conformational epitope. A region of a polypeptide contributing to an epitope can be contiguous amino acids of the polypeptide, forming a linear epitope, or the epitope can be formed from two or more non-contiguous amino acids or regions of the polypeptide, typically called a conformational epitope. The epitope may or may not be a three-dimensional surface feature of the antigen. In some aspects, a NELL2 epitope is a three-dimensional surface feature of a NELL2 polypeptide. In some aspects, a NELL2 epitope is linear feature of a NELL2 polypeptide.

An antibody binds “an epitope” or “essentially the same epitope” or “the same epitope” as a reference antibody, when the two antibodies recognize identical, overlapping, or adjacent epitopes in a three-dimensional space. The most widely used and rapid methods for determining whether two antibodies bind to identical, overlapping, or adjacent epitopes in a three-dimensional space are competition assays, which can be configured in a number of different formats, for example, using either labeled antigen or labeled antibody. In some assays, the antigen is immobilized on a 96-well plate, or expressed on a cell surface, and the ability of unlabeled antibodies to block the binding of labeled antibodies to antigen is measured using a detectable signal, e.g., radioactive, fluorescent or enzyme labels.

The term “compete” when used in the context of anti-NELL2 antibodies that compete for the same epitope or binding site on a NELL2 target protein or peptide thereof means competition as determined by an assay in which the antibody under study, or binding fragment thereof, prevents, blocks, or inhibits the specific binding of a reference molecule (e.g., a reference ligand, or reference antigen binding protein, such as a reference antibody) to a common antigen (e.g., NELL2 or a fragment thereof). Numerous types of competitive binding assays can be used to determine if a test antibody competes with a reference antibody for binding to NELL2 (e.g., human NELL2). Examples of assays that can be employed include solid phase direct or indirect radioimmunoassay (RIA); solid phase direct or indirect enzyme immunoassay (EIA); sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619); solid phase direct labeled assay; solid phase direct labeled sandwich assay (see, e.g., Harlow and Lane, 1988, Antibodies, A Laboratory Manual, Cold Spring Harbor Press); solid phase direct label RIA using labeled iodine (1125 label) (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of a purified antigen (e.g., NELL2) bound to a solid surface, or cells bearing either of an unlabeled test antigen binding protein (e.g., test anti-NELL2 antibody) or a labeled reference antigen binding protein (e.g., reference anti-NELL2 antibody). Competitive inhibition can be measured by determining the amount of label bound to the solid surface or cells in the presence of a known amount of the test antigen binding protein. Usually the test antigen binding protein is present in excess. Antibodies identified by competition assay (competing antibodies) include antibodies binding to the same epitope as the reference antibody and/or antibodies binding to an adjacent epitope sufficiently proximal to the epitope bound by the reference antibody causing steric hindrance to occur. Additional details regarding methods for determining competitive binding are described herein. Usually, when a competing antibody protein is present in excess, it will inhibit specific binding of a reference antibody to a common antigen by at least 15%, or at least 20%, for example, without limitation, 25%, 30%, 35%, 40%, 45%, 50%, 55%, 60%, 65%, 70% or 75% or greater, as well as percent amounts between the amounts stated. In some aspects, binding can be inhibited by at least 80%, 85%, 90%, 95%, 96% or 97%, 98%, 99% or more.

As used herein, the term “blocking” antibody or an “antagonist” antibody refers to an antibody that prevents, inhibits, blocks, or reduces biological or functional activity of the antigen to which it binds. Blocking antibodies or antagonist antibodies can substantially or completely prevent, inhibit, block, or reduce the biological activity or function of the antigen. For example, a blocking anti-NELL2 antibody can prevent, inhibit, block, or reduce the binding interaction between NELL2 and Robo3, thus preventing, blocking, inhibiting, or reducing the growth-promoting functions associated with the NELL2/Robo3 interaction. The terms block, inhibit, and neutralize are used interchangeably herein and refer to the ability of the anti-NELL2 antibodies to prevent or otherwise disrupt or reduce the NELL2/Robo3 interaction.

Slit-Robo signaling plays an important role in axon guidance (Blockus, H., and Chedotal, A. (2016). Development 143, 3037-3044). Binding of Slit ligand to Robo1/2 receptor on the surface of axons results in repulsion of the axons (Brose, K., et al. (1999) Cell 96, 795-806; Long, H., et al. (2004) Neuron 42, 213-223; and Zhou, F., et al. (2020) Cell reports 33, 108332). Mammalian Robo3, a divergent member of the Robo family, does not bind Slits (Mambetisaeva, E. T., et al. (2005) Developmental dynamics: an official publication of the American Association of Anatomists 233, 41-51; and Zelina, P., et al. (2014) Neuron 84, 1258-1272), but is, nonetheless, required for midline crossing of commissural axons (Chen, Z., et al. (2008) Neuron 58, 325-332; and Sabatier, C., et al. (2004) Cell 117, 157-169). The search for a ligand for Robo3 identified NELL2 (neural epidermal growth factor-like-like 2) (Jaworski, A., et al. Science 350, 961-965). NELL2 is a secreted glycoprotein predominantly expressed in neural tissues and was shown to repel axons through Robo3 (Jaworski, A., et al. Science 350, 961-965). A chicken homolog of NELL2, Nel (neural EGF-like), also functions as an inhibitory axon guidance cue (Jiang, Y., et al. (2009) Molecular and cellular neurosciences 41, 113-119; and Nakamura, R., et al. (2012) J Biol Chem 287, 3282-3291.

The BAF (Brg/Brahma-associated factors) or mammalian SWI/SNF complexes are ATP-dependent chromatin remodeling complexes implicated in a variety of cancers (Hodges, C., et al. (2016) The Many Roles of BAF (mSWI/SNF) and PBAF Complexes in Cancer. Cold Spring Harbor perspectives in medicine 6; Kadoch, C., and Crabtree, G. R. (2013) Cell 153, 71-85; and St Pierre, R., and Kadoch, C. (2017) Curr Opin Genet Dev 42, 56-67). The 15 BAF subunits encoded by 29 genes are collectively mutated in more than 20% of human cancers (Hodges, C., et al. (2016) The Many Roles of BAF (mSWI/SNF) and PBAF Complexes in Cancer. Cold Spring Harbor perspectives in medicine 6; Kadoch, C., and Crabtree, G. R. (2013) Cell 153, 71-85; and St Pierre, R., and Kadoch, C. (2017) Curr Opin Genet Dev 42, 56-67). Recurrent inactivating mutations of important BAF subunits in cancers suggested a tumor suppressor role for the BAF complexes. In addition, cancer-specific alteration or re-targeting of BAF complexes was proposed to play a tumor-promoting role in certain cancer types (Boulay, G., et al. (2017) Cell 171, 1-16; and Kadoch, C., and Crabtree, G. R. (2013) Cell 153, 71-85). Despite the importance of BAF complexes in chromatin remodeling and cancers, the signaling pathways regulating these complexes are poorly understood.

Ewing sarcoma is a cancer of bone and soft tissue in children that is characterized by a chromosomal translocation generating a fusion between EWS and an Ets family transcription factor, most commonly FLI1 (Jedlicka, P. (2010) Int J Clin Exp Pathol 3, 338-347; Lawlor, E. R., and Sorensen, P. H. (2015) Critical reviews in oncogenesis 20, 155-171; Lessnick, S. L., and Ladanyi, M. (2012) Annu Rev Pathol 7, 145-159; Mackintosh, C., et al. (2010) Cancer Biol Ther 9, 655-667; and Toomey, E. C., et al. (2010) Oncogene 29, 4504-4516). EWS-FLI1 fusion accounts for 85% of the cases. EWS-FLI1 regulates the expression of a number of genes important for cell proliferation and tumor progression (Hancock, J. D., and Lessnick, S. L. (2008) Cell Cycle 7, 250-256)), can transform mouse cells (Gonzalez, I., et al. (2007) J Mol Med (Berl) 85, 1015-1029; and May, W. A., et al. (1993) Proc Natl Acad Sci USA 90, 5752-5756), and is required for proliferation and tumorigenicity of Ewing sarcoma cells. Therefore, EWS-FLI1 is considered a causative oncoprotein for Ewing sarcoma. Concerning the mechanism of gene activation by EWS-FLI1, it was recently demonstrated that EWS-FLI1 recruits the BAF complexes to the target genes to activate their expression (Boulay, G., et al. (2017) Cell 171, 1-16), suggesting a tumor-promoting role for the BAF complexes in this cancer.

BAF chromatin remodeling complexes play important roles in chromatin regulation and cancer. The Examples herein describe Ewing sarcoma cells that are dependent on the autocrine signaling mediated by NELL2, a secreted glycoprotein that has been characterized as an axon guidance molecule. NELL2 uses Robo3 as the receptor to transmit important growth signaling. NELL2 signaling inhibits cdc42 and upregulates the BAF complexes and EWS-FLI1 transcriptional output. The results described herein demonstrate that cdc42 is a negative regulator of the BAF complexes, inducing actin polymerization and complex disassembly. Furthermore, NELL2^{high}CD133^{high}EWS-FLI1^{high} and NELL2^{low}CD133^{low} EWS-FLI1^{low} populations were identified in Ewing sarcoma which display phenotypes consistent with high and low NELL2 signaling, respectively. The results also show that NELL2, CD133, and EWS-FLI1 positively regulate each other and upregulate the BAF complexes and cell proliferation in Ewing sarcoma. These results reveal a signaling pathway regulating important chromatin remodeling complexes and cancer cell proliferation.

Ewing sarcoma. Ewing sarcoma is a rare bone tumor that occurs most often in adolescents. It can also arise outside of the bone in soft tissue. This type of tumor is associated with chromosomal translocation resulting in the fusion of the gene EWS and an Ets family transcription factor, most commonly FLI1.

Ewing sarcomas tend to affect the middle part of the arms and legs. These tumors also commonly affect flat bones such as the pelvis, chest wall, and spinal column. These tumors are often aggressive and may spread (metastasize) to additional areas of the body, e.g., to other bones and the lungs.

Ewing sarcoma affects males more often than females. It may affect individuals of any age, but most often occurs in individuals between 10 and 20 years of age. Ewing sarcoma is the second most common primary bone tumor in children and accounts for approximately 2% of childhood cancer diagnoses. The annual incidence of Ewing sarcoma is 2.93 children per 1,000,000. Approximately 200-250 children and adolescents in the United States are diagnosed with a tumor in the Ewing family of tumors each year. Two-thirds will be long-term survivors (more than five years). The tumor occurs with greater frequency in Caucasians. It is extremely rare in African Americans and Asians.

Individuals with a tumor in the Ewing family of tumors are treated with multiple anticancer drugs (chemotherapy) in combination with surgical procedures and/or radiation. Surgical removal of the malignancy and affected tissue or radiation is used to treat the primary tumor site. Chemotherapy kills cancer cells in the primary site as well as hidden cancer cells that may have spread into other areas of the body. Generally, systemic chemotherapy is administered first, followed by surgery or radiation. Surgery or radiation therapy without adjuvant chemotherapy has been far less effective than combination therapy. Radiation is often used to treat tumors that are inoperable and sometimes for metastatic disease. Chemotherapy drugs often used to treat individuals with Ewing sarcoma include doxorubicin, vincristine, cyclophosphamide, dactinomycin, ifosfamide, and etoposide.

Neuroblastoma. Neuroblastoma is a solid cancerous tumor that begins in the nerve cells outside the brain of infants and young children. It can start in the nerve tissue near the spine in the neck, chest, abdomen, or pelvis, but it most often begins in the adrenal glands. The adrenal glands are located on top of both kidneys. These glands make hormones that help control body functions, such as heart rate and blood pressure.

Neuroblasts are immature nerve cells found in unborn babies. Normal neuroblasts mature into nerve cells or adrenal medulla cells, which are cells found in the center of the adrenal gland. Neuroblastoma forms when neuroblasts don't mature properly.

Neuroblastoma develops most often in infants and children younger than 5. It can form before the baby is born and can sometimes be found during a prenatal (before birth) ultrasound. Most often, neuroblastoma is found after the cancer has spread to other parts of the body, such as the lymph nodes, which are tiny, bean-shaped organs that help fight infection, liver, lungs, bones, and bone marrow, which is the spongy, red tissue in the inner part of large bones.

Approximately 1% to 2% of children with neuroblastoma have a family history of the disease. In most patients with a family history of neuroblastoma, germline mutations in the anaplastic lymphoma kinase (ALK) gene are detected. These genetic mutations result in abnormal ALK activation. Activating ALK mutations have also been identified in DNA from neuroblastoma tumors in patients without a family history and in a subset of patients, ALK amplification is found in neuroblastoma tumors.

Each year, about 800 children ages 0 to 14 are diagnosed with neuroblastoma in the United States. Neuroblastoma accounts for 6% of all childhood cancers in the United States. Almost 90% of neuroblastoma is found in children younger than 5. The average age of diagnosis is between 1 and 2 years old. The disease is the most commonly diagnosed cancer in children younger than 1. The 5-year survival rate for neuroblastoma is 81%. However, a child's survival rate depends on many factors, particularly the risk grouping of the tumor. For children with low-risk neuroblastoma, the 5-year survival rate is higher than 95%. For children with intermediate-risk neuroblastoma, the 5-year survival rate is between 90% and 95%. For high-risk neuroblastoma, the-5-year survival rate is around 40% to 50%. See Stages and Groups for information on risk groupings. About 2 out of 3 children with neuroblastoma are diagnosed with the disease after it has spread to the lymph nodes or to other parts of the body.

The treatment of recurrent neuroblastoma depends on where the tumor recurred, the previous treatment, tumor biology and its possible gene mutations. While there are treatments that work well for patients with low-risk and intermediate-risk disease who have a recurrence where the original tumor started, recurrent high-risk neuroblastoma remains difficult to cure completely. Neuroblastoma comes back in approximately 50% of children with high-risk disease. Current treatments include chemotherapy, immunotherapy, tyrosine kinase inhibitors, aurora kinase inhibitors, and angiogenesis inhibitors.

The advantage of the disclosed compositions and methods is that the disclosed compositions and methods allow molecularly targeted therapy for Ewing sarcoma, neuroblastoma, medulloblastoma, and glioblastoma, which will be more effective with fewer side effects compared with the state-of-the-art chemotherapy, which use non-specific cytotoxic drugs.

### Compositions

Anti-NELL2 antibodies. Disclosed herein are anti-NELL2 antibodies or binding fragments thereof. Disclosed herein are anti-NELL2 antibodies or binding fragments thereof that bind to NELL2. Disclosed herein are anti-NELL2 antibodies or binding fragments thereof that bind to NELL2 and block or inhibit the immune suppressive function of the NELL2/Robo3 interaction (e.g., block or inhibit the binding of NELL2 to Robo3). Disclosed herein are anti-NELL2 antibodies or binding fragments thereof useful in the treating cancer and inhibiting or preventing tumor or cancer metastases.

The anti-NELL2 antibodies can be of the IgG, IgM, IgA, IgD, and IgE Ig classes, as well as polypeptides comprising one or more antibody CDR domains that retain antigen binding activity. Illustratively, the anti-NELL2 antibodies can be chimeric, affinity matured, humanized, or human antibodies. In some aspects, the anti-NELL2 antibodies can be monoclonal antibodies. In some aspects, the monoclonal anti-NELL2 antibody can be a humanized antibody. By known means and as described herein, polyclonal or monoclonal antibodies, antibody fragments, binding domains and CDRs (including engineered forms of any of the foregoing) can be created that are specific for NELL2 antigen, one or more of its respective epitopes, or conjugates of any of the foregoing, whether such antigens or epitopes are isolated from natural sources or are synthetic derivatives or variants of the natural protein.

Also disclosed herein are compositions comprising anti-NELL2 antibodies. In some aspects, the antibodies disclosed herein can be isolated antibodies. Examples of the CDR sequences and heavy or light chain variable region sequences of anti-NELL2 antibodies are shown in Table 3.

Disclosed herein are isolated antibodies comprising a light chain variable region and a heavy chain variable region. In some aspects, the light chain variable region can comprise a complementarity determining region light chain 1 (CDRL1) amino acid sequence of SEQ ID NO: 1; a complementarity determining region light chain 2 (CDRL2) amino acid sequence of SEQ ID NO: 2; and a complementarity determining region light chain 3 (CDRL3) amino acid sequence of SEQ ID NO: 3. In some aspects, the heavy chain variable region can comprise a complementarity determining region heavy chain 1 (CDRH1) amino acid sequence of SEQ ID NO: 11; a complementarity determining region heavy chain 2 (CDRH2) amino acid sequence of SEQ ID NO: 12; and a complementarity determining region heavy chain 3 (CDRH3) amino acid sequence of SEQ ID NO: 13.

Also disclosed herein, are isolated antibodies comprising a light chain variable region amino acid sequence of SEQ ID NO: 4 and a heavy chain variable region amino acid sequence of SEQ ID NO: 14.

In some aspects, any of the antibodies disclosed herein can comprise a light chain variable region amino acid sequence comprising SEQ ID NO: 4. In some aspects, any of the antibodies disclosed herein can comprise a heavy chain variable region amino acid sequence comprising SEQ ID NO: 14. In some aspects, a light chain variable region has an amino acid sequence that is at least 90% identical to amino acid sequence SEQ ID NO: 4. In some aspects, a heavy chain variable region has an amino acid sequence that is at least 90% identical to amino acid sequence SEQ ID NO: 14.

Disclosed herein are isolated antibodies comprising a light chain variable region and a heavy chain variable region, wherein the light chain variable region comprises a complementarity determining region light chain 1 (CDRL1) amino acid sequence of SEQ ID NO: 1; a determining region light chain 2 (CDRL2) amino acid sequence of SEQ ID NO: 2; and a determining region light chain 3 (CDRL3) amino acid sequence of SEQ ID NO: 3; and wherein the heavy chain variable region comprises a complementarity determining region heavy chain 1 (CDRH1) amino acid sequence of SEQ ID NO: 11: a complementarity determining region heavy chain 2 (CDRH2) amino acid sequence of SEQ ID NO: 12; and a complementarity determining region heavy chain 3 (CDRH3) amino acid sequence of SEQ ID NO: 13, wherein one or more of the CDRL1, CDRL2, CDRL3, CDRH1, CDRH2, or CDRH3 comprise 1, 2, 3, 4, or 5 amino acid substitutions. In some aspects, the amino acid substitutions are conservative amino acid substitutions.

Disclosed herein are isolated antibodies comprising a light chain variable region amino acid sequence of SEQ ID NO: 4 and a heavy chain variable region amino acid sequence of SEQ ID NO: 14, wherein the isolated antibody comprises 1, 2, 3, 4, or 5 amino acid substitutions in the light or heavy chain variable region amino acid sequences. In some aspects, the amino acid substitutions are conservative amino acid substitutions.

Disclosed herein are isolated antibodies comprising a light chain variable region and a heavy chain variable region. In some aspects, the light chain variable region can comprise a complementarity determining region light chain 1 (CDRL1) amino acid sequence of SEQ ID NO: 96; a complementarity determining region light chain 2 (CDRL2) amino acid sequence of SEQ ID NO: 97; and a complementarity determining region light chain 3 (CDRL3) amino acid sequence of SEQ ID NO: 98. In some aspects, the heavy chain variable region can comprise a complementarity determining region heavy chain 1 (CDRH1) amino acid sequence of SEQ ID NO: 86; a complementarity determining region heavy chain 2 (CDRH2) amino acid sequence of SEQ ID NO: 87; and a complementarity determining region heavy chain 3 (CDRH3) amino acid sequence of SEQ ID NO: 88.

Also disclosed herein, are isolated antibodies comprising a light chain variable region amino acid sequence of SEQ ID NO: 99 and a heavy chain variable region amino acid sequence of SEQ ID NO: 89.

In some aspects, any of the antibodies disclosed herein can comprise a light chain variable region amino acid sequence comprising SEQ ID NO: 99. In some aspects, any of the antibodies disclosed herein can comprise a heavy chain variable region amino acid sequence comprising SEQ ID NO: 89. In some aspects, a light chain variable region has an amino acid sequence that is at least 90% identical to amino acid sequence SEQ ID NO: 99. In some aspects, a heavy chain variable region has an amino acid sequence that is at least 90% identical to amino acid sequence SEQ ID NO: 89.

Disclosed herein are isolated antibodies comprising a light chain variable region and a heavy chain variable region, wherein the light chain variable region comprises a complementarity determining region light chain 1 (CDRL1) amino acid sequence of SEQ ID NO: 96; a determining region light chain 2 (CDRL2) amino acid sequence of SEQ ID NO: 97; and a determining region light chain 3 (CDRL3) amino acid sequence of SEQ ID NO: 98; and wherein the heavy chain variable region comprises a complementarity determining region heavy chain 1 (CDRH1) amino acid sequence of SEQ ID NO: 86; a complementarity determining region heavy chain 2 (CDRH2) amino acid sequence of SEQ ID NO: 87; and a complementarity determining region heavy chain 3 (CDRH3) amino acid sequence of SEQ ID NO: 88, wherein one or more of the CDRL1, CDRL2, CDRL3, CDRH1, CDRH2, or CDRH3 comprise 1, 2, 3, 4, or 5 amino acid substitutions. In some aspects, the amino acid substitutions are conservative amino acid substitutions.

Disclosed herein are isolated antibodies comprising a light chain variable region amino acid sequence of SEQ ID NO: 99 and a heavy chain variable region amino acid sequence of SEQ ID NO: 89, wherein the isolated antibody comprises 1, 2, 3, 4, or 5 amino acid substitutions in the light or heavy chain variable region amino acid sequences. In some aspects, the amino acid substitutions are conservative amino acid substitutions.

TABLE 3
Exemplary Amino Acid Sequences of anti-NELL2 antibody

| SEQ ID | | |
|---|---|---|
| NO: | Sequence | Description |
| 1 | RASQEISDYLT | CDRL1, |
| | | IMGT |
| 2 | AASTLDS | CDRL2, |
| | | IMGT |
| 3 | LQYASFPLT | CDRL3, |
| | | IMGT |
| 4 | DIQMTQSPSSLSASLGERVSLTCRASQEIS | Light |
| | DYLTWLQQKPDGTIKRLIYAASTLDSGVPK | Chain |
| | RFSGSRSGSDYSLTISSLESEDFADYYCLQ | Variable |
| | YASFPLTFGAGTKLELK | Domain |
| | | IMGT |
| 5 | MRVPAHVFGFLLLWFPGTRCDIQMTQSPSS | Light |
| | LSASLGERVSLTCRASQEISDYLTWLQQKP | Chain |
| | DGTIKRLIYAASTLDSGVPKRFSGSRSGSD | Variable |
| | YSLTISSLESEDFADYYCLQYASFPLTFGA | Domain, |
| | GTKLELK | with signal |
| | | peptide |
| | | (underline), |
| | | IMGT |
| 6 | CGGGCAAGTCAGGAAATTAGTGATTACTTAACC | CDRL1, |
| | | IMGT |
| 7 | GCCGCATCCACTTTAGATTCT | CDRL2, |
| | | IMGT |
| 8 | CTACAATATGCTAGTTTTCCGCTCACG | CDRL3, |
| | | IMGT |
| 9 | GACATCCAGATGACCCAGTCTCCATCCTCCTTAT | Light |
| | CTGCCTCTCTGGGAGAAAGAGTCAGTCTCACTTGT | Chain |
| | CGGGCAAGTCAGGAAATTAGTGATTACTTAACCTG | Variable |
| | GCTTCAGCAGAAACCAGATGGAACTATTAAACGCC | Domain, |
| | TGATCTACGCCGCATCCACTTTAGATTCTGGTGTC | IMGT |
| | CCAAAAAGGTTCAGTGGCAGTAGGTCTGGGTCAGA | |
| | TTATTCTCTCACCATCAGCAGCCTTGAGTCTGAAGA | |
| | TTTTGCAGACTATTACTGTCTACAATATGCTAGTTT | |
| | TCCGCTCACGTTCGGTGCTGGGACTAAGCTGGAGC | |
| | TGAAA | |
| 10 | ATGAGGGTTCCTGCTCACGTTTTTGGCTTCTTGTTG | Light |
| | CTCTGGTTTCCAGGTACCAGATGTGACATCCAGAT | Chain |
| | GACCCAGTCTCCATCCTCCTTATCTGCCTCTCTGGG | Variable |
| | AGAAAGAGTCAGTCTCACTTGTCGGGCAAGTCAGG | Domain, |
| | AAATTAGTGATTACTTAACCTGGCTTCAGCAGAAA | with signal |
| | CCAGATGGAACTATTAAACGCCTGATCTACGCCGC | sequence |
| | ATCCACTTTAGATTCTGGTGTCCCAAAAAGGTTCAG | (underline), |
| | TGGCAGTAGGTCTGGGTCAGATTATTCTCTCACCAT | IMGT |
| | CAGCAGCCTTGAGTCTGAAGATTTTGCAGACTATTA | |
| | CTGTCTACAATATGCTAGTTTTCCGCTCACGTTCGGT | |
| | GCTGGGACTAAGCTGGAGCTGAAA | |
| 11 | SFGMH | CDRH1, |
| | | IMGT |
| 12 | YVSGGSSTIYYADTVKG | CDRH2, |
| | | IMGT |
| 13 | SFSLLRLPVYFDF | CDRH3, |
| | | IMGT |
| 14 | DVQLVESGGGLVQPGGSRKLSCAASG | Heavy |
| | FTFSSFGMHWVRQAPEKGLEWVAYVS | Chain |
| | GGSSTIYYADTVKGRFTISRDNPKNT | Variable |
| | LFLQMTSLRSEDTAMYYCARSFSLLR | Domain, |
| | LPVYFDFWGQGTTLTVSS | IMGT |
| 15 | MDSRLNLVFLVLILKGVQCDVQLVESG | Heavy |
| | GGLVQPGGSRKLSCAASGFTFSSFGMH | Chain |
| | WVRQAPEKGLEWVAYVSGGSSTIYYAD | Variable |
| | TVKGRFTISRDNPKNTLFLQMTSLRSE | Domain, |
| | DTAMYYCARSFSLLRLPVYFDFWGQGT | with signal |
| | TLTVSS | peptide |
| | | (underline), |
| | | IMGT |
| 16 | AGCTTTGGAATGCAC | CDRH1, |
| | | IMGT |
| 17 | TACGTTAGTGGTGGCAGTAGTACCATCTACTAT | CDRH2, |
| | GCAGACACAGTGAAGGGC | IMGT |
| 106 | TCCTTCTCATTACTGCGACTACCGGTCTACTTTG | CDRH3, |
| | ACTTC | IMGT |
| 18 | ATGGACTCCAGGCTCAATTTAGTTTTCCTTG | Heavy |
| | TCCTTATTTTAAAAGGTGTCCAGTGTGATGT | Chain |
| | GCAGCTGGTGGAGTCTGGGGGAGGCTTAGTG | Variable |
| | CAGCCTGGAGGGTCCCGGAAACTCTCCTGTG | Domain, |
| | CAGCCTCTGGATTCACTTTCAGTAGCTTTGG | nucleic |
| | AATGCACTGGGTTCGTCAGGCTCCAGAGAAG | acid |
| | GGGCTGGAGTGGGTCGCATACGTTAGTGGTG | sequence, |
| | GCAGTAGTACCATCTACTATGCAGACACAGT | with signal |
| | GAAGGGCCGATTCACCATCTCCAGAGACAAT | sequence |
| | CCCAAGAACACCCTGTTCCTGCAAATGACCA | (underline), |
| | GTCTCAGGTCTGAGGACACGGCCATGTATTAC | IMGT |
| | TGTGCAAGATCCTTCTCATTACTGCGACTACC | |
| | GGTCTACTTTGACTTCTGGGGCCAAGGCACCA | |
| | CTCTCACAGTCTCCTCA | |
| 19 | GATGTGCAGCTGGTGGAGTCTGGGGGAGGCTT | Heavy |
| | AGTGCAGCCTGGAGGGTCCCGGAAACTCTCCT | Chain |
| | GTGCAGCCTCTGGATTCACTTTCAGTAGCTTTG | Variable |
| | GAATGCACTGGGTTCGTCAGGCTCCAGAGAAG | Domain, |
| | GGGCTGGAGTGGGTCGCATACGTTAGTGGTG | IMGT |
| | GCAGTAGTACCATCTACTATGCAGACACAGT | |
| | GAAGGGCCGATTCACCATCTCCAGAGACAAT | |
| | CCCAAGAACACCCTGTTCCTGCAAATGACCA | |
| | GTCTCAGGTCTGAGGACACGGCCATGTATTAC | |
| | TGTGCAAGATCCTTCTCATTACTGCGACTACC | |
| | GGTCTACTTTGACTTCTGGGGCCAAGGCACCA | |
| | CTCTCACAGTCTCCTCA | |
| 80 | ATGGACTCCAGGCTCAATTTAGTTTTCCTTGTC | Heavy |
| | CTTATTTTAAAAGGTGTCCAGTGTGATGTGCAG | Chain |
| | CTGGTGGAGTCTGGGGGAGGCTTAGTGCAGCC | Variable |
| | TGGAGGGTCCCGGAAACTCTCCTGTGCAGCCT | Domain, |
| | CTGGATTCACTTTCAGTAGCTTTGGAATGCAC | F10 |
| | TGGGTTCGTCAGGCTCCAGAGAAGGGGCTGG | |
| | AGTGGGTCGCATACGTTAGTGGTGGCAGTAGT | |
| | ACCATCTACTATGCAGACACAGTGAAGGGCCG | |
| | ATTCACCATCTCCAGAGACAATCCCAAGAACA | |
| | CCCTGTTCCTGCAAATGACCAGTCTCAGGTCTG | |
| | AGGACACGGCCATGTATTACTGTGCAAGATCCT | |
| | TCTCATTACTGCGACTACCGGTCTACTTTGACT | |
| | TCTGGGGCCAAGGCACCACTCTCACAGTCTCCT | |
| | CA | |
| 81 | ATGGACTCCAGGCTCAATTTAGTTTTCCTTGTCC | Heavy |
| | TTATTTTAAAAGGTGTCCAGTGTGATGTGCAGCT | Chain |
| | GGTGGAGTCTGGGGGAGGCTTAGTGCAGCCTGG | Variable |
| | AGGGTCCCGGAAACTCTCCTGTGCAGCCTCTGGA | Domain, |
| | TTCACTTTCAGTAGCTTTGGAATGCACTGGGTTC | H03 |
| | GTCAGGCTCCAGAGAAGGGGCTGGAGTGGGTCG | |
| | CATACGTTAGTGGTGGCAGTAGTACCATCTACTA | |
| | TGCAGACACAGTGAAGGGCCGATTCACCATCTCC | |
| | AGAGACAATCCCAAGAACACCCTGTTCCTGCAAA | |
| | TGACCAGTCTCAGGTCTGAGGACACGGCCATGTA | |
| | TTACTGTGCAAGATCCTTCTCATTACTGCGACTAC | |
| | CGGTCTACTTTGACTTCTGGGGCCAAGGCACCAC | |
| | TCTCACAGTCTCCTCA | |
| 82 | TGGACTCCAGGCTCAATTTAGTTTTCCTTGTCCTT | Heavy |
| | ATTTTAAAAGGTGTCCAGTGTGATGTGCAGCTGG | Chain |
| | TGGAGTCTGGGGGAGGCTTAGTGCAGCCTGGAGG | Variable |
| | GTCCCGGAAACTCTCCTGTGCAGCCTCTGGATTCA | Domain, |
| | CTTTCAGTAGCTTTGGAATGCACTGGGTTCGTCAG | B11 |
| | GCTCCAGAGAAGGGGCTGGAGTGGGTCGCATACG | |
| | TTAGTGGTGGCAGTAGTACCATCTACTATGCAGAC | |
| | ACAGTGAAGGGCCGATTCACCATCTCCAGAGACA | |
| | ATCCCAAGAACACCCTGTTCCTGCAAATGACCAGT | |
| | CTCAGGTCTGAGGACACGGCCATGTATTACTGTGC | |
| | AAGATCCTTCTCATTACTGCGACTACCGGTCTAC | |
| | TTTGACTTCTGGGGCCAAGGCACCACTCTCACAGT | |
| | CTCCTCA | |
| 83 | GACTCCAGGCTCAATTTAGTTTTCCTTGTCCTTATTTT | Heavy |
| | AAAAGGTGTCCAGTGTGATGTGCAGCTGGTGGAGTC | Chain |
| | TGGGGGAGGCTTAGTGCAGCCTGGAGGGTCCCGGAAA | Variable |
| | CTCTCCTGTGCAGCCTCTGGATTCACTTTCAGTAGCTTT | Domain, |
| | GGAATGCACTGGGTTCGTCAGGCTCCAGAGAAGGGGCT | H04 |
| | GGAGTGGGTCGCATACGTTAGTGGTGGCAGTAGTACC | |
| | ATCTACTATGCAGACACAGTGAAGGGCCGATTCACCAT | |
| | CTCCAGAGACAATCCCAAGAACACCCTGTTCCTGCAAA | |
| | TGACCAGTCTCAGGTCTGAGGACACGGCCATGTATTAC | |
| | TGTGCAAGATCCTTCTCATTACTGCGACTACCGGTCTAC | |
| | TTTGACTTCTGGGGCCAAGGCACCACTCTCACAGTCTCC | |
| | TCA | |
| 84 | ATTTAGTTTTCCTTGTCCTTATTTTAAAAGGTGTCC | Heavy |
| | AGTGTGATGTGCAGCTGGTGGAGTCTGGGGGAGG | Chain |
| | CTTAGTGCAGCCTGGAGGGTCCCGGAAACTCTCCT | Variable |
| | GTGCAGCCTCTGGATTCACTTTCAGTAGCTTTGGA | Domain, |
| | ATGCACTGGGTTCGTCAGGCTCCAGAGAAGGGGCT | C04 |
| | GGAGTGGGTCGCATACGTTAGTGGTGGCAGTAGTA | |
| | CCATCTACTATGCAGACACAGTGAAGGGCCGATTCA | |
| | CCATCTCCAGAGACAATCCCAAGAACACCCTGTT | |
| | CCTGCAAATGACCAGTCTCAGGTCTGAGGACACGGC | |
| | CATGTATTACTGTGCAAGATCCTTCTCATTACTGCG | |
| | ACTACCGGTCTACTTTGACTTCTGGGGCCAAGGCA | |
| | CCACTCTCACAGTCTCCTCA | |
| 85 | ATGGACTCCAGGCTCAATTTAGTTTTCCTTGTCCTT | Heavy |
| | ATTTTAAAAGGTGTCCAGTGTGATGTGCAGCTGGT | Chain |
| | GGAGTCTGGGGGAGGCTTAGTGCAGCCTGGAGGG | Variable |
| | TCCCGGAAACTCTCCTGTGCAGCCTCTGGATTCAC | Domain |
| | TTTCAGTAGCTTTGGAATGCACTGGGTTCGTCAGG | |
| | CTCCAGAGAAGGGGCTGGAGTGGGTCGCATACG | |
| | TTAGTGGTGGCAGTAGTACCATCTACTATGCAGA | |
| | CACAGTGAAGGGCCGATTCACCATCTCCAGAGAC | |
| | AATCCCAAGAACACCCTGTTCCTGCAAATGACCA | |
| | GTCTCAGGTCTGAGGACACGGCCATGTATTAC | |
| | TGTGCAAGATCCTTCTCATTACTGCGACTACCGG | |
| | TCTACTTTGACTTCTGGGGCCAAGGCACCACTCT | |
| | CACAGTCTCCTCA | |
| 86 | DYYLY | CDRH1, |
| | | IMGT |
| 87 | YISNGGDITYYPDTVKG | CDRH2, |
| | | IMGT |
| 88 | GRVRSYYGNPDAMDF | CHRH3, |
| | | IMGT |
| 89 | EVKLVESGGGLVQPGGSLKLSCATSGFTF | Heavy |
| | SDYYLYWVRQTPEKRLEWVAYISNGGDI | Chain |
| | TYYPDTVKGRFAISRDNAKNTLYLQMSRL | Variable |
| | KSEDTAMYYCARGRVRSYYGNPDAMDF | Domain |
| | WGQGTSVTVSS | IMGT |
| 90 | MNLGLSLIFLVLVLKGVQCEVKLVESGG | Heavy |
| | GLVQPGGSLKLSCATSGFTFSDYYLYWV | Chain |
| | RQTPEKRLEWVAYISNGGDITYYPDTVK | Variable |
| | GRFAISRDNAKNTLYLQMSRLKSEDTAM | Domain, |
| | YYCARGRVRSYYGNPDAMDFWGQGTSV | with signal |
| | TVSS | peptide |
| | | (underline), |
| | | IMGT |
| 91 | GACTATTACTTATAT | CDRH1, |
| | | IMGT |
| 92 | TACATTAGCAATGGTGGTGATATTACCTATT | CDRH2, |
| | ATCCAGACACTGTAAAGGGC | IMGT |
| 93 | GGAAGAGTCCGATCCTACTATGGTAACCCC | CDRH3, |
| | GATGCTATGGACTTC | IMGT |
| 94 | ATGAACTTGGGGCTCAGCTTGATTTTCCTT | Heavy |
| | GTCCTTGTTTTAAAAGGTGTCCAGTGTGAA | Chain |
| | GTTAAGCTGGTGGAGTCTGGGGGAGGCTT | Variable |
| | AGTGCAGCCTGGAGGGTCCCTGAAACTCT | Domain, |
| | CCTGTGCTACCTCTGGATTCACTTTCAGTG | nucleic |
| | ACTATTACTTATATTGGGTTCGCCAGACTC | acid |
| | CAGAGAAGAGGCTGGAGTGGGTCGCATAC | sequence, |
| | ATTAGCAATGGTGGTGATATTACCTATTAT | with signal |
| | CCAGACACTGTAAAGGGCCGATTCGCCAT | sequence |
| | CTCCAGAGACAATGCCAAGAACACCCTGT | (underline), |
| | ACCTGCAAATGAGCCGTCTGAAGTCTGAG | IMGT |
| | GACACAGCCATGTATTACTGTGCAAGGGG | |
| | AAGAGTCCGATCCTACTATGGTAACCCCG | |
| | ATGCTATGGACTTCTGGGGTCAAGGAACC | |
| | TCAGTCACCGTCTCCTCA | |
| 95 | GAAGTTAAGCTGGTGGAGTCTGGGGGAGG | Heavy |
| | CTTAGTGCAGCCTGGAGGGTCCCTGAAACT | Chain |
| | CTCCTGTGCTACCTCTGGATTCACTTTCAGT | Variable |
| | GACTATTACTTATATTGGGTTCGCCAGACT | Domain, |
| | CCAGAGAAGAGGCTGGAGTGGGTCGCATA | IMGT |
| | CATTAGCAATGGTGGTGATATTACCTATTA | |
| | TCCAGACACTGTAAAGGGCCGATTCGCCAT | |
| | CTCCAGAGACAATGCCAAGAACACCCTGTA | |
| | CCTGCAAATGAGCCGTCTGAAGTCTGAGGA | |
| | CACAGCCATGTATTACTGTGCAAGGGGAAG | |
| | AGTCCGATCCTACTATGGTAACCCCGATGCT | |
| | ATGGACTTCTGGGGTCAAGGAACCTCAGTCA | |
| | CCGTCTCCTCA | |
| 96 | RSSQSLVHSNGNTYLH | CDRL1, |
| | | IMGT |
| 97 | KVSKRFS | CDRL2, |
| | | IMGT |
| 98 | SQSIYVPHT | CDRL3, |
| | | IMGT |
| 99 | DVVMTQTPLSLPVSLGDQASISCRSSQSLVHSN | Light |
| | GNTYLHWYLQKPGQSPKLLIYKVSKRFSGVPD | Chain |
| | RFSGSGSGTDFTLKITRVEAEDLGVYFCSQSIY | Variable |
| | VPHTFGGGTKLEIK | Domain |
| | | IMGT |
| 100 | MKLPVRLLVLMFWIPASSSDVVMTQTPLSLPV | Light |
| | SLGDQASISCRSSQSLVHSNGNTYLHWYLQKP | Chain |
| | GQSPKLLIYKVSKRFSGVPDRFSGSGSGTDFTL | Variable |
| | KITRVEAEDLGVYFCSQSIYVPHTFGGGTKLEIK | Domain, |
| | | with signal |
| | | peptide |
| | | (underline), |
| | | IMGT |
| 101 | AGATCTAGTCAGAGCCTTGTACACAGTAATG | CDRL1, |
| | GAAACACCTATTTACAT | IMGT |
| 102 | AAAGTTTCCAAACGATTTTCT | CDRL2, |
| | | IMGT |
| 103 | TCTCAAAGTATATATGTTCCGCACACG | CDRL3, |
| | | IMGT |
| 104 | GATGTTGTGATGACCCAAACTCCACTCTCCCTG | Light |
| | CCTGTCAGTCTTGGAGATCAGGCCTCCATCTCT | Chain |
| | TGCAGATCTAGTCAGAGCCTTGTACACAGTAAT | Variable |
| | GGAAACACCTATTTACATTGGTACCTGCAGAAG | Domain, |
| | CCAGGCCAGTCTCCAAAGCTCCTGATCTACAAA | IMGT |
| | GTTTCCAAACGATTTTCTGGGGTCCCAGACAGG | |
| | TTCAGTGGCAGTGGATCAGGGACAGATTTCACA | |
| | CTCAAGATCACCAGAGTGGAGGCTGAGGATCTG | |
| | GGAGTTTATTTCTGCTCTCAAAGTATATATGTTC | |
| | CGCACACGTTCGGAGGGGGGACCAAGCTGGAAA | |
| | TAAAA | |
| 105 | ATGAAGTTGCCTGTTAGGCTGTTGGTGCTGATGT | Light |
| | TCTGGATTCCTGCTTCCAGCAGTGATGTTGTGAT | Chain |
| | GACCCAAACTCCACTCTCCCTGCCTGTCAGTCTT | Variable |
| | GGAGATCAGGCCTCCATCTCTTGCAGATCTAGTC | Domain, |
| | AGAGCCTTGTACACAGTAATGGAAACACCTATTT | with signal |
| | ACATTGGTACCTGCAGAAGCCAGGCCAGTCTCCA | sequence |
| | AAGCTCCTGATCTACAAAGTTTCCAAACGATTTT | (underline), |
| | CTGGGGTCCCAGACAGGTTCAGTGGCAGTGGAT | IMGT |
| | CAGGGACAGATTTCACACTCAAGATCACCAGAG | |
| | TGGAGGCTGAGGATCTGGGAGTTTATTTCTGCTC | |
| | TCAAAGTATATATGTTCCGCACACGTTCGGAGGG | |
| | GGGACCAAGCTGGAAATAAAA | |
| 106 | GACTATTACTTATAT | CDRH1, |
| | | IMGT |
| 107 | TACATTAGCAATGGTGGTGATATTACCTATTAT | CDRH2, |
| | CCAGACACTGTAAAGGGC | IMGT |
| 108 | GGAAGAGTCCGATCCTACTATGGTAACCCCGA | CHRH3, |
| | TGCTATGGACTTC | IMGT |
| 109 | AGATCTAGTCAGAGCCTTGTACACAGTAATGG | CDRL1, |
| | AAACACCTATTTACAT | IMGT |
| 110 | AAAGTTTCCAAACGATTTTCT | CDRL2, |
| | | IMGT |
| 111 | TCTCAAAGTATATATGTTCCGCACACG | CDRL3, |
| | | IMGT |

The CDRs disclosed herein may also include variants. Generally, the amino acid identity between individual variant CDRs is at least 90%, 91%, 92%, 93%, 94%, 95%, 96%, 97%, 98%, 99%, or 100%. Thus, a “variant CDR” is one with the specified identity to the parent or reference CDR of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR. For example, a “variant CDR” can be a sequence that contains 1, 2, 3, 4 or 5 amino acid changes as compared to the parent or reference CDR of the invention, and shares or improves biological function, specificity and/or activity of the parent CDR.

In some aspects, the disclosed antibodies can comprise any of CDR sequences disclosed herein and can include a single amino acid change as compared to the parent or reference CDR. In some aspects, any of the CDR sequences disclosed herein can include at least two amino acid changes as compared to the parent or reference CDR. In some aspects, the amino acid change can be a change from a cysteine residue to another amino acid. In some aspects, the amino acid change can be a change from a glycine residue to another amino acid. The amino acid identity between individual variant CDRs can be at least 60%, 65%, 70%, 75%, 80%, 85%, 90%, 91%, 92%, 93%, 94%, 95%, 96%, 97%, 98%, 99% or 100%. Thus, a “variant CDR” can be one with the specified identity to the parent CDR of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR. For example, the parent CDR sequence can be one or more of SEQ ID NOs: 1, 2, 3, 11, 12, 13, 86, 87, 88, 96, 87 and/or 99. The variant CDR sequence can be at least 60%, 65%, 70%, 75%, 80%, 85%, 90%, 91%, 92%, 93%, 94%, 95%, 96%, 97%, 98%, 99% or 100% identical to any one of SEQ ID NOs: 1, 2, 3, 11, 12, 13, 86, 87, 88, 96, 97, and/or 98. The variant CDR sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR.

As discussed herein, minor variations in the amino acid sequences of any of the antibodies disclosed herein are contemplated as being encompassed by the instant disclosure, providing that the variations in the amino acid sequence maintains at least 75%, more preferably at least 80%, 90%, 95%, and most preferably 99% sequence identity to the parent sequence. In some aspects, conservative amino acid replacements are contemplated. Conservative replacements are those that take place within a family of amino acids that are related in their side chains. Genetically encoded amino acids are generally divided into families: (1) acidic-aspartate, glutamate; (2) basic=lysine, arginine, histidine; (3) non-polar-alanine, valine, leucine, isoleucine, proline, phenylalanine, methionine, tryptophan; and (4) uncharged polar-glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are known to one of ordinary skill in the art.

In some aspects, amino acid substitutions can be those which: (1) reduce susceptibility to proteolysis, (2) reduce susceptibility to oxidation, (3) alter binding affinity for forming protein complexes, (4) alter binding affinities, and (4) confer or modify other physiocochemical or functional properties of such analogs. In some aspects, single or multiple amino acid substitutions (preferably conservative amino acid substitutions) may be made in the non-CDR sequence of the heavy chain, the light chain or both. In some aspects, one or more amino acid substitutions can be made in one or more of the CDR sequences of the heavy chain, the light chain or both.

Many methods have been developed for chemical labeling and enhancement of the properties of antibodies and their common fragments, including the Fab and F(ab)2 fragments. Somewhat selective reduction of some antibody disulfide bonds has been previously achieved, yielding antibodies and antibody fragments that can be labeled at defined sites, enhancing their utility and properties. Selective reduction of the two hinge disulfide bonds present in F(ab)2 fragments using mild reduction has been useful. In some aspects, cysteine and methionine can be susceptible to rapid oxidation, which can negatively influence the cleavage of protecting groups during synthesis and the subsequent peptide purification. In some instances, cysteine residues in peptides used for antibody production can affect the avidity of the antibody, because free cysteines are uncommon in vivo and therefore may not be recognized by the native peptide structure. In some aspects, the disclosed antibodies and fragments thereof comprise a sequence where a cysteine reside outside of the CDR (e.g. in the non-CDR sequence of the heavy chain, the light chain or both) is substituted. In some aspects, cysteine can be replaced with serine and methionine replaced with norleucine (Nle). Multiple cysteines on a peptide or in one of the disclosed antibodies or fragments thereof may be susceptible to forming disulfide linkages unless a reducing agent such as dithiothreitol (DTT) is added to the buffer or the cysteines can be replaced with serine residues.

While the site or region for introducing an amino acid sequence variation is predetermined, the mutation per se need not be predetermined. For example, in order to optimize the performance of a mutation at a given site, random mutagenesis may be conducted at the target codon or region and the expressed antigen binding protein CDR variants screened for the optimal combination of desired activity. Techniques for making substitution mutations at predetermined sites in DNA having a known sequence are well known, for example, M13 primer mutagenesis and PCR mutagenesis. Screening of the mutants is done using assays of antigen binding protein activities as described herein.

Amino acid substitutions are typically of single residues: insertions usually will be on the order of from about one (1) to about twenty (20) amino acid residues, although considerably larger insertions may be tolerated. Deletions range from about one (1) to about twenty (20) amino acid residues, although in some cases deletions may be much larger.

Substitutions, deletions, insertions or any combination thereof may be used to arrive at a final derivative or variant. Generally these changes are done on a few amino acids to minimize the alteration of the molecule, particularly the immunogenicity and specificity of the antigen binding protein. However, larger changes may be tolerated in certain circumstances.

By “Fab” or “Fab region” as used herein is meant the polypeptide that comprises the VH, CH1, VL, and CL immunoglobulin domains. Fab may refer to this region in isolation, or this region in the context of a full length antibody, antibody fragment or Fab fusion protein, or any other antibody embodiments as outlined herein.

By “Fv” or “Fv fragment” or “Fv region” as used herein is meant a polypeptide that comprises the VL and VH domains of a single antibody.

By “framework” as used herein is meant the region of an antibody variable domain exclusive of those regions defined as CDRs. Each antibody variable domain framework can be further subdivided into the contiguous regions separated by the CDRs (FR1, FR2, FR3 and FR4).

Disclosed herein are isolated antibodies comprising a light chain variable region, wherein the light chain variable region comprises a variant complementarity determining region light chain 1 (CDRL1), positions 24-34 of SEQ ID NO: 4. In some aspects, the variant CDRL1 can comprise one or two amino acid substitutions. Also, disclosed herein are isolated antibodies comprising a light chain variable region, wherein the light chain variable region comprises a variant CDRL2, positions 50-56 of SEQ ID NO: 4. In some aspects, the variant CDRL2 can comprise one or two amino acid substitutions. Further disclosed herein are isolated antibodies comprising a light chain variable region, wherein the light chain variable region comprises a variant CDRL3, 89-97 positions of SEQ ID NO: 4. In some aspects, the variant CDRL3 can comprise one or two amino acid substitutions. In some aspects, the amino acid substitutions are conservative amino acid substitutions.

Disclosed herein are isolated antibodies comprising a heavy chain variable region, wherein the heavy chain variable region comprises a variant complementarity determining region heavy chain 1 (CDRH1), positions 31-35 of SEQ ID NO: 14. In some aspects, the variant CDRH1 can comprise one or two amino acid substitutions. Also disclosed herein are isolated antibodies comprising a heavy chain variable region, wherein the heavy chain variable region comprises a variant CDRH2, positions 50-66 of SEQ ID NO: 14. In some aspects, the variant CDRH2 can comprise one or two amino acid substitutions. Further disclosed herein are isolated antibodies comprising a heavy chain variable region, wherein the heavy chain variable region can comprise a variant CDRH3, positions 99-111 of SEQ ID NO: 14. In some aspects, the variant CDRH3 can comprise one or two amino acid substitutions. In some aspects, the amino acid substitutions are conservative amino acid substitutions.

Disclosed herein are isolated antibodies comprising a light chain variable region, wherein the light chain variable region comprises a variant complementarity determining region light chain 1 (CDRL1), positions 24-39 of SEQ ID NO: 99. In some aspects, the variant CDRL1 can comprise one or two amino acid substitutions. Also, disclosed herein are isolated antibodies comprising a light chain variable region, wherein the light chain variable region comprises a variant CDRL2, positions 55-61 of SEQ ID NO: 99. In some aspects, the variant CDRL2 can comprise one or two amino acid substitutions. Further disclosed herein are isolated antibodies comprising a light chain variable region, wherein the light chain variable region comprises a variant CDRL3, 94-102 positions of SEQ ID NO: 99. In some aspects, the variant CDRL3 can comprise one or two amino acid substitutions. In some aspects, the amino acid substitutions are conservative amino acid substitutions.

Disclosed herein are isolated antibodies comprising a heavy chain variable region, wherein the heavy chain variable region comprises a variant complementarity determining region heavy chain 1 (CDRH1), positions 31-35 of SEQ ID NO: 89. In some aspects, the variant CDRH1 can comprise one or two amino acid substitutions. Also disclosed herein are isolated antibodies comprising a heavy chain variable region, wherein the heavy chain variable region comprises a variant CDRH2, positions 50-66 of SEQ ID NO: 89. In some aspects, the variant CDRH2 can comprise one or two amino acid substitutions. Further disclosed herein are isolated antibodies comprising a heavy chain variable region, wherein the heavy chain variable region can comprise a variant CDRH3, positions 99-113 of SEQ ID NO: 89. In some aspects, the variant CDRH3 can comprise one or two amino acid substitutions. In some aspects, the amino acid substitutions are conservative amino acid substitutions.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are as described in Table 1. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

In some aspects, the CDRs can be defined according to the Kabat definition. In some aspects, the CDRs can be defined according to the IMGT definition.

In some aspects, the antibodies disclosed herein can be recombinantly engineered, chimerized, or humanized. In some aspects, the antibodies disclosed herein can be affinity matured or human antibodies. In some aspects, the antibodies disclosed herein can be a Fab, an Fab′, an F(ab)2, a Fv, a scFv, a diabody or fragments thereof. In some aspects, the antibody can be a monoclonal antibody. In some aspects, the monoclonal antibodies can be humanized or chimeric forms thereof. In some aspects, the monoclonal antibody can be a humanized antibody. By known means and as described herein, polyclonal or monoclonal antibodies, antibody fragments, binding domains and CDRs (including engineered forms of any of the foregoing) may be created that are specific for NELL2 antigen, one or more of its respective epitopes, or conjugates of any of the foregoing, whether such antigens or epitopes are isolated from natural sources or are synthetic derivatives or variants of the natural protein.

A monoclonal antibody is a single, clonal species of antibody wherein every antibody molecule recognizes the same epitope because all antibody producing cells are derived from a single, antibody-producing B-lymphocyte (or other clonal cell, such as a cell that recombinantly expresses the antibody molecule). The methods for generating monoclonal antibodies (MAbs) generally begin along the same lines as those for preparing polyclonal antibodies. In some aspects, rodents such as mice and rats are used in generating monoclonal antibodies. In some aspects, rabbit, sheep, or frog cells are used in generating monoclonal antibodies. The use of rats is well known and may provide certain advantages. Mice (e.g., BALB/c mice) are routinely used and generally give a high percentage of stable fusions. Hybridoma technology as used in monoclonal antibody production involves the fusion of a single, antibody-producing B lymphocyte isolated from a mouse previously immunized with a NELL2 protein or peptide with an immortalized cell, e.g., a mouse cell line. This technology provides a method to propagate a single antibody-producing cell for an indefinite number of generations, such that unlimited quantities of structurally identical antibodies having the same antigen or epitope specificity, i.e., monoclonal antibodies, may be produced.

Methods have been developed to replace light and heavy chain constant domains of the monoclonal antibody with analogous domains of human origin, leaving the variable regions of the foreign antibody intact. Alternatively, “fully human” monoclonal antibodies are produced in mice or rats that are transgenic for human immunoglobulin genes. Methods have also been developed to convert variable domains of monoclonal antibodies to more human form by recombinantly constructing antibody variable domains having both rodent and human amino acid sequences. In “humanized” monoclonal antibodies, only the hypervariable CDRs are derived from non-human (e.g., mouse, rat, chicken, llama, etc.) monoclonal antibodies, and the framework regions are derived from human antibody amino acid sequences. The replacement of amino acid sequences in the antibody that are characteristic of rodents with amino acid sequences found in the corresponding positions of human antibodies reduces the likelihood of adverse immune reaction to foreign protein during therapeutic use in humans. A hybridoma or other cell producing an antibody may also be subject to genetic mutation or other changes, which may or may not alter the binding specificity of antibodies produced by the hybridoma.

Engineered antibodies may be created using monoclonal and other antibodies and recombinant DNA technology to produce other antibodies or chimeric molecules that retain the antigen or epitope binding specificity of the original antibody, i.e., the molecule has a specific binding domain. Such techniques may involve introducing DNA encoding the immunoglobulin variable region or the CDRs of an antibody into the genetic material for the framework regions, constant regions, or constant regions plus framework regions, of a different antibody. See, for instance, U.S. Pat. Nos. 5,091,513 and 6,881,557, which are incorporated herein by reference.

By known means as described herein, polyclonal or monoclonal antibodies, antibody fragments having binding activity, binding domains and CDRs (including engineered forms of any of the foregoing), may be created that specifically bind to NELL2 protein, one or more of its respective epitopes, or conjugates of any of the foregoing, whether such antigens or epitopes are isolated from natural sources or are synthetic derivatives or variants of the natural compounds.

Antibodies may be produced from any animal source, including birds and mammals. In some aspects, the antibodies can be ovine, murine (e.g., mouse and rat), rabbit, goat, guinea pig, camel, horse, or chicken. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546, which is incorporated herein by reference. These techniques are further described in Marks et al., 1992, Bio/Technol., 10:779-783; Stemmer, 1994, Nature, 370:389-391; Gram et al., 1992, Proc. Natl. Acad. Sci. USA, 89:3576-3580; Barbas et al., 1994, Proc. Natl. Acad. Sci. USA, 91:3809-3813; and Schier et al., 1996, Gene, 169(2): 147-155.

Methods for producing polyclonal antibodies in various animal species, as well as for producing monoclonal antibodies of various types, including humanized, chimeric, and fully human, are well known in the art and are highly reproducible. For example, the following U.S. patents provide descriptions of such methods and are herein incorporated by reference: U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861, 155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; 6,891,024; 7,407,659; and 8,178,098.

In some aspects, the antibody can be a single chain antibody. In some aspects, the antibody can be linked to a detectable label. In some aspects, antibody can be a monovalent or a bivalent antibody.

In some aspects, the antibodies disclosed herein can be an IgG, an IgM, an IgA, an IgD, or an IgE antibody or antigen binding fragment thereof. In some aspects, the antibodies can be of the IgG, IgM, IgA, IgD, and IgE Ig classes or a genetically modified IgG class antibody, as well as polypeptides comprising one or more antibody CDR regions that retain antigen binding activity. In some aspects, the antibody can be an IgG class of antibody. In some aspects, the IgG class antibody can be an IgG1, IgG2, IgG3, or IgG4 class antibody.

Also disclosed herein are isolated nucleotide molecules comprising a nucleotide sequence selected from SEQ ID NOs: 9 or 10 and/or a nucleotide sequence selected from SEQ ID NO: 19, or 18, respectively. In some aspects, the isolated nucleotide sequence encoding an anti-NELL2 VL domain can be a nucleotide sequence that is at least 90-98% identical to the nucleotide sequence of SEQ ID NOs: 9 or 10. In some aspects, the isolated nucleotide sequence encoding an anti-NELL2 VH domain can be nucleotide sequence that is at least 90-98% identical to the nucleotide sequence of SEQ ID NOs: 19 or 18. In some aspects, the nucleotide sequences encoding the VL and/or the VH domains can be 90%, 91%, 92%, 93%, 94%, 95%, 96%, 97%, 98%, 99%, or more identical to SEQ ID NOs: 9 or 10, or SEQ ID NOs: 19 or 18, respectively. These nucleotide sequences encode VL domains and VH domains that at least in the context of a bivalent antibody, specifically bind to NELL2.

Also disclosed herein are isolated nucleotide molecules comprising a nucleotide sequence selected from SEQ ID NOs: 104 or 105 and/or a nucleotide sequence selected from SEQ ID NO: 94, or 95, respectively. In some aspects, the isolated nucleotide sequence encoding an anti-NELL2 VL domain can be a nucleotide sequence that is at least 90-98% identical to the nucleotide sequence of SEQ ID NOs: 104 or 105. In some aspects, the isolated nucleotide sequence encoding an anti-NELL2 VH domain can be nucleotide sequence that is at least 90-98% identical to the nucleotide sequence of SEQ ID NOs: 94 or 95. In some aspects, the nucleotide sequences encoding the VL and/or the VH domains can be 90%, 91%, 92%, 93%, 94%, 95%, 96%, 97%, 98%, 99%, or more identical to SEQ ID NOs: 104 or 105, or SEQ ID NOs: 94 or 95, respectively. These nucleotide sequences encode VL domains and VH domains that at least in the context of a bivalent antibody, specifically bind to NELL2.

Further disclosed herein are isolated polynucleotides or sets of isolated polynucleotides. In some aspects, the isolated polynucleotides or sets of isolated polynucleotides can comprise at least one nucleic acid sequence that encodes any of isolated antibodies disclosed herein. Optionally, the isolated polynucleotide or set of isolated polynucleotides can be cDNA.

Disclosed herein are vectors or sets of vectors. In some aspects, the vectors or sets of vectors can comprise one or more of the polynucleotides or sets of polynucleotides disclosed herein. Optionally, the vectors or sets of vectors can be selected from the group consisting of a plasmid, a viral vector, a non-episomal mammalian vector, an expression vector, and a recombinant expression vector.

Disclosed herein are isolated cells. In some aspects, the isolated cells can comprise a polynucleotide or set of polynucleotides disclosed herein. In some aspects, the isolated cells can optionally be selected from a hybridoma, a Chinese Hamster Ovary (CHO) cell, or a HEK293 cell. Also disclosed herein are methods of producing any of the isolated antibodies disclosed herein. In some aspects, the methods can comprise culturing a host cell under conditions suitable for expressing the isolated antibody wherein the host cell comprises a polynucleotide or set of polynucleotides as disclosed herein, and purifying the isolated antibody.

In some aspects, the antibody can be a bispecific antibody. Unifying two antigen binding sites of different specificity into a single construct, bispecific antibodies have the ability to bring together two discreet antigens with exquisite specificity and therefore have great potential as therapeutic agents. Bispecific antibodies were originally made by fusing two hybridomas, each capable of producing a different immunoglobulin. Bispecific antibodies can also be produced by joining two scFv antibody fragments while omitting the Fc portion present in full immunoglobulins. Each scFv unit in such constructs can contain one variable domain from each of the heavy (V_{H}) and light (V_{L}) antibody chains, joined with one another via a synthetic polypeptide linker, the latter often being genetically engineered so as to be minimally immunogenic while remaining maximally resistant to proteolysis. Respective scFv units may be joined by a number of known techniques, including incorporation of a short (usually less than 10 amino acids) polypeptide spacer bridging the two scFv units, thereby creating a bispecific single chain antibody. The resulting bispecific single chain antibody is therefore a species containing two V_{H}/V_{L} pairs of different specificity on a single polypeptide chain, in which the V_{H} and V_{L} domains in a respective scFv unit are separated by a polypeptide linker long enough to allow intramolecular association between these two domains, such that the so-formed scFv units are contiguously tethered to one another through a polypeptide spacer kept short enough to prevent unwanted association between, for example, the V_{H} domain of one scFv unit and the V_{L} of the other scFv unit.

Examples of antibody fragments suitable for use include, without limitation: (i) the Fab fragment, consisting of V_{L}, V_{H}, C_{L}, and C_{H1} domains; (ii) the “Fd” fragment consisting of the V_{H} and C_{H1} domains; (iii) the “Fv” fragment consisting of the V_{L} and V_{H} domains of a single antibody; (iv) the “dAb” fragment, which consists of a V_{H} domain; (v) isolated CDR regions; (vi) F(ab′)2 fragments, a bivalent fragment comprising two linked Fab fragments; (vii) single chain Fv molecules (“scFv”), in which a V_{H} domain and a V_{L} domain are linked by a peptide linker that allows the two domains to associate to form a binding domain; (viii) bi-specific single chain Fv dimers (see U.S. Pat. No. 5,091,513); and (ix) diabodies, multivalent, or multispecific fragments constructed by gene fusion (U.S. Patent Appln. Pub. No. 20050214860). Fv, scFv, or diabody molecules may be stabilized by the incorporation of disulfide bridges linking the V_{H} and V_{L} domains. Minibodies comprising a scFv joined to a C_{H3} domain (Hu et al., 1996, Cancer Res., 56:3055-3061) may also be useful. In addition, antibody-like binding peptidomimetics are also contemplated. “Antibody like binding peptidomimetics” (ABiPs), which are peptides that act as pared-down antibodies and have certain advantages of longer serum half-life as well as less cumbersome synthesis methods, have been reported by Liu et al., 2003, Cell Mol. Biol., 49:209-216.

Animals may be inoculated with an antigen, such as a NELL2 polypeptide or peptide to generate an immune response and produce antibodies specific for the NELL2 polypeptide. Frequently, an antigen is bound or conjugated to another molecule to enhance the immune response. As used herein, a conjugate can be any peptide, polypeptide, protein, or non-proteinaceous substance bound to an antigen that is used to elicit an immune response in an animal. Antibodies produced in an animal in response to antigen inoculation comprise a variety of non-identical molecules (polyclonal antibodies) made from a variety of individual antibody producing B lymphocytes. A polyclonal antibody is a mixed population of antibody species, each of which may recognize a different epitope on the same antigen. Given the correct conditions for polyclonal antibody production in an animal, most of the antibodies in the animal's serum will recognize the collective epitopes on the antigenic compound to which the animal has been immunized. This specificity is further enhanced by affinity purification to select only those antibodies that recognize the antigen or epitope of interest.

The antibodies described herein directed to NELL2 have the ability to neutralize, block, inhibit, or counteract the effects of NELL2 binding to Robo3 regardless of the animal species, monoclonal cell line or other source of the antibody. Certain animal species may be less preferable for generating therapeutic antibodies because they may be more likely to cause an immune or allergic response due to activation of the complement system through the “Fc” portion of the antibody. However, whole antibodies may be enzymatically digested into the “Fc” (complement binding) fragment, and into peptide fragments having the binding domains or CDRs. Removal of the Fc portion reduces the likelihood that this antibody fragment will elicit an undesirable immunological response and, thus, antibodies without an Fc portion may be preferential for prophylactic or therapeutic treatments. As described above, antibodies may also be constructed so as to be chimeric, humanized, or partially or fully human, so as to reduce or eliminate potential adverse immunological effects resulting from administering to an animal an antibody that has been produced in, or has amino acid sequences from, another species.

In some aspects, the antibodies disclosed herein bind to human NELL2. In some aspects, the antibodies disclosed herein bind to human NELL2. In some aspects, the antibody selectively binds to NELL2 and inhibits binding of NELL2 to Robo3. In some aspects, the antibody selectively binds to human NELL2 and inhibits binding of human NELL2 to human Robo3.

The term “specifically binds” (or “immunospecifically binds”) is not intended to indicate that an antibody binds exclusively to its intended target. Rather, an antibody “specifically binds” if its affinity for its intended target is about, for example, 5-fold greater when compared to its affinity for a non-target molecule. Suitably there is no significant cross-reaction or cross-binding with undesired substances. The affinity of the antibody will, for example, be at least about 5-fold, such as 10-fold, such as 25-fold, especially 50-fold, and particularly 100-fold or more, greater for a target molecule than its affinity for a non-target molecule. In some aspects, specific binding between an antibody or other binding agent and an antigen means a binding affinity of at least 10⁶ M⁻¹. Antibodies may, for example, bind with affinities of at least about 10⁷ M⁻¹, such as between about 10⁸ M⁻¹ to about 10⁹ M⁻¹, about 10⁹ M⁻¹ to about 10¹⁰ M⁻¹, or about 10⁻¹⁰M⁻¹ to about 10¹¹ M⁻¹. Antibodies may, for example, bind with an EC50 of 1000 nM or less, 500 nM or less, 100 nM or less, 50 nM or less, 10 nM or less, 1 nM or less. In some aspects, the antibodies can bind with an EC50 of about 500 μg/ml, 100 μg/ml, 75 μg/ml, 70 μg/ml, 65 μg/ml, 60 μg/ml, 55 μg/ml, 54 μg/ml, 53 μg/ml, 52 μg/ml, 51 μg/ml, 50 μg/ml or less.

In some aspects, the antibodies described herein comprise a heavy chain variable region, wherein the heavy chain variable region comprises one or more complementarity determining region (CDRHs) CDRH1, CDRH2 and CDRH3, wherein the CDRH1, CDRH2 and CDRH3 comprise the amino acid sequences of SEQ ID NO: 11, SEQ ID NO: 12, and SEQ ID NO: 13, respectively, wherein at least one of CDRH1, CDRH2 and CDRH3 further comprise 0), 1, 2, 3, 4, or 5 conservative amino acid substitutions; and/or a light chain variable region comprising one or more complementarity determining region (CDRLs) CDRL1, CDRL2 and CDRL3, wherein CDRL1, CDRL2 and CDRL3 comprise the amino acid sequences of SEQ ID NO: 1, SEQ ID NO: 2, and SEQ ID NO: 3, respectively, wherein at least one of CDRL1, CDRL2 and CDRL3 further comprise 0), 1, 2, 3, 4, or 5 conservative amino acid substitutions. In some aspects, the amino acid substitutions are conservative amino acid substitutions.

In some aspects, the antibodies described herein comprise a heavy chain variable region, wherein the heavy chain variable region comprises one or more complementarity determining region (CDRHs) CDRH1, CDRH2 and CDRH3, wherein the CDRH1, CDRH2 and CDRH3 comprise the amino acid sequences of SEQ ID NO: 86, SEQ ID NO: 87, and SEQ ID NO: 88, respectively, wherein at least one of CDRH1, CDRH2 and CDRH3 further comprise 0), 1, 2, 3, 4, or 5 conservative amino acid substitutions; and/or a light chain variable region comprising one or more complementarity determining region (CDRLs) CDRL1, CDRL2 and CDRL3, wherein CDRL1, CDRL2 and CDRL3 comprise the amino acid sequences of SEQ ID NO: 96, SEQ ID NO: 97, and SEQ ID NO: 98, respectively, wherein at least one of CDRL1, CDRL2 and CDRL3 further comprise 0), 1, 2, 3, 4, or 5 amino acid substitutions. In some aspects, the amino acid substitutions are conservative amino acid substitutions.

In some aspects, the antibodies disclosed herein can specifically bind to an epitope of NELL2. In some aspects, the epitope of NELL2 can be in a region comprising EGF-like repeats. In some aspects, the amino acid sequence of the EGF-like repeats is:
GYDFCSERHNCMENSICRNLNDRAVCSCRDGFRALREDNAYCEDIDECAEGR HYCRENTMCVNTPGSFMCICKTGYIRIDDYSCTEHDECITNQHNCDENALCFNTVGG HNCVCKPGYTGNGTTCKAFCKDGCRNGGACIAANVCACPQGFTGPSCETDIDECSD GFVQCDSRANCINLPGWYHCECRDGYHDNGMFSPSGESCEDIDECGTGRHSCANDTI CFNLDGGYDCRCPHGKNCT (SEQ ID NO: 79).

In some aspects, the antibodies disclosed herein can prevent, inhibit or block NELL2 binding to Robo3. In some aspects, the antibodies disclosed herein can inhibit the binding of human NELL2 to human Robo3. In some aspects, the antibodies disclosed herein can prevent, inhibit or block the interaction of human NELL2 to human Robo3. In some aspects, the antibodies disclosed herein can increase active cdc42 levels. As used herein, the term “active cdc42” is defined as GTP-bound cdc42. cdc42 exists in two forms: GTP-bound active form and GDP-bound inactive form. In some aspects, the antibodies disclosed herein can reduce GTP-bound cdc42 levels. In some aspects, the antibodies disclosed herein can prevent, inhibit or block tumor metastases generally and, in some aspects, specifically, for example, prevent, inhibit or block metastatic spread of cancers brain, Ewing sarcoma or neuroblastoma.

Antibody proteins may be recombinant, or synthesized in vitro. It is contemplated that in anti-NELL2 antibody-containing compositions as described herein can comprise between about 0.001 mg and about 10 mg of total antibody polypeptide per ml. Thus, the concentration of antibody protein in a composition can be about, at least about or at most about or equal to 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein). Of this, about, at least about, at most about, or equal to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% may be an antibody that binds NELL2.

Disclosed herein are compositions comprising any of the antibodies or isolated antibodies described herein. In some aspects, the compositions can further comprise at least one pharmaceutically acceptable carrier or diluent.

In some aspects, the compositions described herein can comprise a detectable label or reporter. An antibody or an immunological portion of an antibody that retains binding activity, can be chemically conjugated to, or recombinantly expressed as, a fusion protein with other proteins. For the purposes as described herein, all such fused proteins are included in the definition of antibodies or an immunological portion of an antibody. In some aspects, antibodies and antibody-like molecules generated against NELL2 or polypeptides that are linked to at least one agent to form an antibody conjugate or payload are encompassed. In order to increase the efficacy of antibody molecules as diagnostic or therapeutic agents, it is conventional to link or covalently bind or complex at least one desired molecule or moiety to the antibody. Such a linked molecule or moiety may be, but is not limited to, at least one effector, detectable label or reporter molecule. Effector molecules comprise molecules having a desired activity, e.g., cytotoxic activity. Non-limiting examples of effector molecules that may be attached to antibodies include toxins, therapeutic enzymes, antibiotics, radio-labeled nucleotides and the like. By contrast, a reporter molecule or detectable label is defined as any moiety that may be detected using an assay. Non-limiting examples of reporter molecules and detectable labels that can be conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin, and the like. Several methods are known in the art for attaching or conjugating an antibody to a conjugate molecule or moiety. Some attachment methods involve the use of a metal chelate complex, employing by way of nonlimiting example, an organic chelating agent such a diethylenetriaminepentaacetic acid anhydride (DTPA); ethylenetriaminetetraacetic acid: N-chloro-p-toluenesulfonamide; and/or tetrachloro-3-6α-diphenylglycouril-3 attached to the antibody. Antibodies, particularly the monoclonal antibodies as described herein, may also be reacted with an enzyme in the presence of a coupling agent such as glutaraldehyde or periodate. Conjugates with fluorescein markers are conventionally prepared in the presence of these coupling agents or by reaction with an isothiocyanate. In some aspects, an anti-NELL2 antibody as described herein, particularly a binding fragment thereof, may be coupled or linked to a compound or substance, such as polyethylene glycol (PEG), to increase its in vivo half-life in plasma, serum, or blood following administration.

In some aspects, the antibodies described herein can be specifically bind to their intended target (e.g., NELL2). In some aspects, the antibodies described herein have no off site binding.

Disclosed herein are hybridomas that can produce any of the isolated antibodies disclosed herein.

### Methods

Disclosed herein are methods for treating a NELL2 positive cancer in a subject. The methods can comprise administering to the subject a therapeutically effective amount of any of the isolated antibodies described herein or any of the compositions described herein. In some aspects, the NELL2 positive can be Ewing sarcoma, neuroblastoma or a brain cancer. In some aspects, the brain cancer can be glioblastoma or medulloblastoma.

Disclosed herein are methods of blocking the binding of NELL2 to or with Robo3 in a subject. In some aspects, the NELL2 levels can be increased compared to a reference sample or a control subject. In some aspects, the method comprises administering therapeutically effective amount of any of the isolated antibodies disclosed herein or any of the compositions disclosed herein. In some aspects, the method comprises administering therapeutically effective amount of any of the anti-NELL2 antibodies disclosed herein. In some aspects, the therapeutically effective amount of the anti-NELL2 antibodies can be an amount to form a complex between the anti-NELL2 antibody and NELL in the subject, thereby reducing, inhibiting or blocking the binding or interaction of NELL2 with Robo3. In some aspects, the methods of blocking the binding of NELL2 to or with Robo3 can result in decreased or reduced levels of NELL2 in the subject compared to the NELL2 levels in the subject before the administration of a therapeutically effective amount of any of the isolated antibodies disclosed herein or any of the compositions disclosed herein. In some aspects, the methods of blocking the binding of NELL2 to or with Robo3 can result in decreased or reduced or no change in the levels of NELL2 in the subject compared to a reference sample or a control subject. In some aspects, the subject can suffer from a cancer. In some aspects, the subject can have a NELL2 positive cancer.

Disclosed herein are methods of increasing active cdc42 (GTP-bound cdc42) levels in a subject. In some aspects, the active cdc42 levels can be decreased or reduced compared to a reference sample or a control subject. In some aspects, the method comprises administering therapeutically effective amount of any of the isolated antibodies disclosed herein or any of the compositions disclosed herein. In some aspects, the method comprises administering therapeutically effective amount of any of the anti-NELL2 antibodies disclosed herein. In some aspects, the therapeutically effective amount of the anti-NELL2 antibodies can be an amount to form a complex between the anti-NELL2 antibody and NELL in the subject, thereby increasing active cdc42. In some aspects, the subject can suffer from a cancer. In some aspects, the subject can have a NELL2 positive cancer.

Also disclosed herein are methods of treating metastatic cancer in a subject or preventing metastasis in a subject at risk of metastasis or metastatic cancer. Also disclosed herein are methods of preventing reoccurrence of a tumor in a subject. In some aspects, the method comprises administering therapeutically effective amount of any of the isolated antibodies disclosed herein or any of the compositions disclosed herein. In some aspects, the cancer can be a cancer of breast, colon, lymphatic system, pancreas, lung, skin (including melanoma), esophagus, head and neck, Ewing sarcoma, neuroblastoma, medulloblatoma, glioblastoma, or stomach. In some aspects, the subject has cancer. In some aspects, the subject has metastatic cancer. In some aspects, the subject can have cancer or be a cancer patient and is at risk for cancer metastasis. In some aspects, administration of any of the antibodies disclosed herein can reduce the number of metastases. In some aspects, administration of any of the antibodies disclosed herein can prevent the occurrence or reoccurrence of metastasis. In some aspects, administration of any of the antibodies disclosed herein can increase the subject's or patient's survival time. In some aspects, administration of any of the antibodies disclosed herein can prevent the reoccurrence of a tumor in the subject. In some aspects, the subject can have a NELL2 positive cancer.

In some aspects, the subject can be identified in need of treatment before the administering step. In some aspects, the antibody can be administered in a pharmaceutically acceptable composition. In some aspects, the antibody can be administered systemically, intravenously, intradermally, intramuscularly, intraperitoneally, subcutaneously or locally into tissues, organs or tumors.

In some aspects, the methods can further comprising administering one or more drugs or therapeutic agents to the subject. Examples of drugs or therapeutic agents that can be administered in combination with any of the antibodies described herein include but are not limited to doxorubicin, etoposide, cyclophosphamide, vincristine, ifosfamide, prednisone, mesalamine, budesonide (Entocort EC), sulfasalazine (Azulfidine®), mesalamine (Asacol, Asacol HD, Lialda, Pentasa®, Apriso), azathioprine (Imuran), sulfazine, Remicade® (infliximab), dexamethasone, mercaptopurine, Acthar®, cyclosporine, tacrolimus, rapamycin, mycophenolate mofetil, rituximab, obinutuzumab, fedratinib, ruxolitinib, idelalisib, alpelisib, duvelisib, copanlisib, ibrutinib, zanubrutinib, or acalabrutinib.

Disclosed herein are antibodies or antigen binding fragments thereof, as described herein (e.g., an antibody that specifically and preferentially binds to NELL2 and blocks or inhibits binding of NELL2 to Robo3) that can be used in treatment methods and administered to treat or prevent cancer or metastatic cancer. Accordingly, provided herein are methods of treating a cancer, and treating metastatic cancer or preventing metastasis in a subject having cancer at risk for metastasis. In some aspects, the methods can comprise administering to a subject a therapeutically effective amount of any of the antibodies described herein or any of the compositions comprising at least one of antibodies as described herein. In some aspects, the drug or therapeutic agent can be an anti-NELL2 antibody or a composition comprising at least one anti-NELL2 antibody.

The compositions described herein can be administered to the subject (e.g., a human patient) in an amount sufficient to delay, reduce, or preferably prevent the onset of clinical disease. Accordingly, in some aspects, the patient can be a human patient. In therapeutic applications, compositions can be administered to a subject (e.g., a human patient) already with or diagnosed with cancer in an amount sufficient to at least partially improve a sign or symptom or to inhibit the progression of (and preferably arrest) the symptoms of the disease or condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the cancer is delayed, hindered, or prevented, or a symptom of the cancer is ameliorated or its frequency can be reduced. One or more of the symptoms can be less severe. Recovery can be accelerated in an individual who has been treated. For example, treatment of cancer or metastatic cancer may involve, for example, a reduction in the size of a tumor, a reduction in the invasiveness of a tumor, reduction in the growth rate of the cancer, or prevention of metastasis. Treatment of cancer or metastatic cancer may also refer to prolonging survival of a subject with cancer. In some aspects, the antibodies described herein can prolong the lifespan of a subject with cancer. In some aspects, the antibodies described herein can reduce or inhibit tumor cell growth.

Treatment of these subjects with an effective amount of at least one of the anti-NELL2 antibodies as described herein can result in binding of one or more of the disclosed antibodies to NELL2, thereby preventing, blocking or inhibiting NELL2 from binding to its antigen, Robo3, and thereby reducing NELL2 levels. Accordingly, the methods as provided are advantageous for a subject who is in need of, capable of benefiting from, or who is desirous of receiving the benefit of, the anti-cancer results or the amelioration of one or more cancer symptoms achieved by the practice of the present methods. A subject's seeking the therapeutic benefits of the methods involving administration of at least one anti-NELL2 antibody in a therapeutically effective amount, or receiving such therapeutic benefits offer advantages to the art. In addition, the present methods offer the further advantages of eliminating or avoiding side effects, adverse outcomes, contraindications, and the like, or reducing the risk or potential for such issues to occur compared with other treatments and treatment modalities.

Cancers for which the present methods are useful include but are not limited to Ewing sarcoma, neuroblastoma, glioblastoma, and medulloblastoma. Other cancers for which the present methods are useful include but are not limited to breast cancer, colon cancer, lymphatic system cancers, pancreatic cancer, lung cancer, skin cancers (including melanoma), esophageal cancer, head and neck cancer, Ewing sarcoma, neuroblastoma, medulloblatoma, glioblastoma and stomach cancer.

The anti-NELL2 antibodies, such as monoclonal anti-NELL2 antibodies, can be used as immunosuppressant agents in a variety of modalities. In some aspects, the methods described herein use the antibodies disclosed herein as immunosuppressant agents, and, thus, comprise contacting a population of cells with a therapeutically effective amount of one or more of the antibodies, or a composition containing one or more of the antibodies, for a time period sufficient to block or inhibit the actions of T lymphocytes. In some aspects, contacting a cell in vivo is accomplished by administering to a subject in need, for example, by intravenous, subcutaneous, intraperitoneal, or intratumoral injection, a therapeutically effective amount of a physiologically tolerable composition comprising an anti-NELL2 antibody as described herein. The antibody may be administered parenterally by injection or by gradual infusion over time. Useful administration and delivery regimens include intravenous, intraperitoneal, oral, intramuscular, subcutaneous, intracavity, transdermal, dermal, peristaltic means, or direct injection into the tissue containing the cells.

Therapeutic compositions comprising antibodies are conventionally administered intravenously, such as by injection of a unit dose, for example. The term “unit dose” when used in reference to a therapeutic composition refers to physically discrete units suitable as unitary dosage for the subject, each unit containing a predetermined quantity of active material calculated to produce the desired therapeutic effect in association with the required diluent, i.e., carrier, or vehicle. The compositions comprising any of the anti-NELL2 antibodies disclosed herein can be administered in a manner compatible with the dosage formulation, and in a therapeutically effective amount. The quantity to be administered depends on the subject to be treated, capacity of the subject's system to utilize the active ingredient, and degree of therapeutic effect desired. Precise amounts of active ingredient required to be administered depend on the judgment of the practitioner and are peculiar to each individual. However, suitable dosage ranges for systemic application are disclosed herein and depend on the route of administration. Suitable regimens for initial and booster administration are also contemplated and may typically involve an initial administration followed by repeated doses at one or more intervals (hours) by a subsequent injection or other administration. In some aspects, multiple administrations can be suitable for maintaining continuously high serum and tissue levels of antibody. Alternatively, continuous intravenous infusion sufficient to maintain concentrations in the blood in the ranges specified for in vivo therapies are contemplated.

It is contemplated that an anti-NELL2 antibody as described herein can be administered systemically or locally to treat disease, such as to inhibit tumor cell growth or to kill cancer cells in cancer patients with locally advanced or metastatic cancers or at risk for metastatic cancers. The antibodies can be administered alone or in combination with anti-proliferative drugs or anticancer drugs. In some aspects, the anti-NELL2 antibodies can be administered to reduce the cancer load in the patient prior to surgery or other procedures. Alternatively, they can be administered at periodic intervals after surgery to ensure that any remaining cancer (e.g., cancer that the surgery failed to eliminate) is reduced in size or growth capacity and/or does not survive. As noted herein, a therapeutically effective amount of an antibody can be a predetermined amount calculated to achieve the desired effect. Thus, the dosage ranges for the administration of an anti-NELL2 antibody are those large enough to produce the desired effect in which the symptoms of tumor cell division and cell cycling are reduced. Optimally, the dosage should not be so large as to cause adverse side effects, such as hyperviscosity syndromes, pulmonary edema, congestive heart failure, neurological effects, and the like. Generally, the dosage will vary with age of, condition of, size and gender of, and extent of the disease in the subject or patient and can be determined by one of skill in the art such as a medical practitioner or clinician. Of course, the dosage may be adjusted by the individual physician in the event of any complication.

In some aspects, the compositions and methods as described herein comprise the administration of an anti-NELL2 antibody as described herein, alone, or in combination with a second or additional drug or therapy. Such drug or therapy may be applied in the treatment of any disease that is associated with NELL2, and in some aspects, the interaction of human NELL2 or with human Robo3. For example, the disease can be a cancer or metastatic cancer. The compositions and methods described herein can comprise at least one anti-NELL2 antibody that preferentially binds to Robo3 protein and has a therapeutic or protective effect in the treatment of a cancer or metastatic cancer, particularly by preventing, reducing, blocking, or inhibiting the NELL2/Robo3 interaction, thereby providing a therapeutic effect and treatment.

The compositions and methods, including combination therapies, have a therapeutic or protective effect and may enhance the therapeutic or protective effect, and/or increase the therapeutic effect of another drug, therapy or therapeutic agent (e.g., anti-cancer or anti-hyperproliferative therapy).

Therapeutic and prophylactic methods and compositions can be provided in a combined amount effective to achieve the desired effect, such as the killing of a cancer cell and/or the inhibition of cellular hyperproliferation; reducing NELL2 levels). This process may involve administering an anti-NELL2 antibody or a binding fragment thereof and a second therapy. The second therapy may or may not have a direct cytotoxic effect. A tissue, tumor, and/or cell can be exposed to one or more compositions or pharmacological formulation(s) comprising one or more of the agents (e.g., an antibody or an anti-cancer agent), or by exposing the tissue, tumor, and/or cell with two or more distinct compositions or formulations, wherein one composition provides, for example, 1) an antibody, 2) an anti-cancer agent, 3) both an antibody and an anti-cancer agent, or 4) two or more antibodies. In some aspects, the second therapy can be also an anti-NELL2 antibody. Also, it is contemplated that such a combination therapy can be used in conjunction with chemotherapy, radiotherapy, surgical therapy, or immunotherapy.

By way of example, the terms “contacted” and “exposed,” when applied to a cell, are used herein to describe a process by which a therapeutic polypeptide, for example, an anti-NELL2 antibody as described herein, is delivered to a target cell or is placed in direct juxtaposition with the target cell, particularly to bind specifically to the target antigen, e.g., NELL2, expressed or highly expressed on neurons, on CD4 T cells, on CD8, T cells, on photoreceptor cells, in blood, and in fallopian tubes. Such binding by a therapeutic anti-NELL2 antibody or binding fragment thereof prevents, blocks, inhibits, or reduces the interaction of NELL2 with Robo3. In some aspects, a chemotherapeutic or radiotherapeutic agent can also be administered or delivered to the subject in conjunction with the anti-NELL2 antibody or binding fragment thereof. To achieve cell killing, for example, one or more agents are delivered to a cell in a combined amount effective to kill the cell or prevent it from dividing.

Any of the anti-NELL2 antibodies disclosed herein may be administered before, during, after, or in various combinations relative to another treatment (e.g., anti-cancer). The administrations may be in intervals ranging from concurrently to minutes to days to weeks before or after one another. In some aspects, in which the antibody is provided to a patient separately from an anti-cancer agent, it would be generally ensured that a significant period of time did not expire between the time of each delivery, such that the administered compounds would still be able to exert an advantageously combined effect for the patient. Illustratively, in such instances, it is contemplated that one may provide a patient with the antibody and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In some aspects, a course of treatment or treatment cycle will last 1-90 days or more (this range includes intervening days and the last day). It is contemplated that one agent may be given on any day of day 1 to day 90 (this such range includes intervening days and the last day) or any combination thereof, and another agent is given on any day of day 1 to day 90 (this such range includes intervening days and the last day) or any combination thereof. Within a single day (24-hour period), the patient may be given one or multiple administrations of the agent(s). Moreover, after a course of treatment, it is contemplated that there may be a period of time at which no second agent (e.g., anti-cancer treatment) is administered. This time period may last, for example, for 1-7 days, and/or 1-5 weeks, and/or 1-12 months or more (this such range includes intervening days and the upper time point), depending on the condition of the patient, such as prognosis, strength, health, etc. Treatment cycles would be repeated as necessary. Various combinations of treatments may be employed. In the representative examples of combination treatment regimens shown below; an antibody, such as an anti-NELL2 antibody or binding fragment thereof is represented by “A” and an anti-cancer therapy is represented by “B”:
A/B/A B/A/B B/B/A A/A/B A/B/B B/A/A A/B/B/B B/A/B/B B/B/B/A B/B/A/B A/A/B/B A/B/A/B A/B/B/A B/B/A/A B/A/B/A B/A/A/B A/A/A/B B/A/A/A A/B/A/A A/A/B/A.

Administration of any antibody or therapy as described herein to a patient will follow general protocols for the administration of such compounds, taking into account the toxicity, if any, of the agents. Therefore, in some aspects there is a step of monitoring adverse events and toxicity, particularly those that may be attributable to combination therapy.

In some aspects, methods are disclosed comprising administering an anti-NELL2 antibody alone or in combination with another agent (e.g., anticancer agent) to a subject in need thereof, i.e., a subject with a cancer or a tumor). Prior to administration of the anti-NELL2 antibody, a sample of the subject's tumor or cancer may be evaluated for the presence or level of NELL2. If the results of such an evaluation reveals that the subject's tumor or cancer is positive for NELL2 or the level of NELL2 is increased compared to a reference sample or prior sample from the same subject, the subject would be selected for treatment based on the likelihood that subject's NELL2+tumor or cancer or disease state or condition would be more amenable to treatment with the anti-NELL2 antibody and treatment may proceed with a more likely beneficial outcome. A medical professional or physician may advise the subject to proceed with the anti-NELL2 antibody treatment method, and the subject may decide to proceed with treatment based on the advice of the medical professional or physician. In addition, during the course of treatment, the subject's tumor or cancer cells or blood cells may be assayed for the presence of NELL2 as a way to monitor the progress or effectiveness of treatment. If the assay shows a change, loss, or decrease, for example, in NELL2 on the subject's tumor or cancer cells or blood cells, a decision may be taken by the medical professional in conjunction with the subject as to whether the treatment should continue or be altered in some fashion, e.g., a higher dosage, the addition of another anti-cancer agent or therapy, and the like.

Chemotherapy. A wide variety of chemotherapeutic agents may be used in accordance with the treatment or therapeutic methods as described herein. The term “chemotherapy” refers to the use of drugs to treat cancer. A “chemotherapeutic agent” connotes a compound or composition that is administered in the treatment of cancer. Such agents or drugs are categorized by their mode of activity within a cell, for example, whether and at what stage they affect the cell cycle and cell growth and proliferation. Alternatively, a chemotherapeutic agent may be characterized based on its ability to directly cross-link DNA, to intercalate into DNA, or to induce chromosomal and mitotic aberrations by affecting nucleic acid synthesis in a cell.

Non-limiting examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabine, navelbine, farnesyl-protein tansferase inhibitors, transplatinum, and pharmaceutically acceptable salts, acids, or derivatives of any of the above.

Radiotherapy. Radiotherapy includes treatments with agents that cause DNA damage. Radiotherapy has been used extensively in cancer and disease treatments and embraces what are commonly known as y-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA itself, on the precursors of DNA, on the replication and repair of DNA, and on the assembly and maintenance of chromosomes. Exemplary dosage ranges for X-rays range from daily doses of 50 to 200 roentgens for prolonged periods of time (3 to 4 weeks) to single doses of 2000 to 6000 roentgens. Dosage ranges for radioisotopes vary widely and depend on the half-life of the isotope, the strength and type of radiation emitted, the uptake by the neoplastic cells, and tolerance of the subject undergoing treatment.

Immunotherapy. In some aspects of the methods, immunotherapies may be used in combination or in conjunction with administration of anti-NELL2 antibodies as described herein. In the context of cancer treatment, immunotherapeutics generally rely on the use of immune effector cells and molecules to target and destroy cancer cells. Rituximab (RITUXAN®) is such an example. In some aspects, checkpoint inhibitors can also be administered in combination, including ipilimumab. The anti-NELL2 antibodies can be administered in combination with anti-PD-1 or anti-PD-L1 inhibitors, such as antibodies against PD-L1, which include atezolizumab, durvalumab, or avelumab, or antibodies against PD-1, including nivolumab, pembrolizumab, or pidilizumab. In addition, one or more of the anti-NELL2 antibodies as described herein may be administered in combination with each other. In some aspects, the anti-NELL2 antibodies can also be administered in combination with NELL2 inhibitors. The antibody alone may serve as an effector of therapy or it may recruit other cells to actually affect cell killing or suppress the immune system. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target, e.g., the PD-1 on T-cells/PD-L1 on tumor cells interaction. Various effector cells include cytotoxic T cells and natural killer (NK) cells.

In some aspects of immunotherapy, the tumor cell must bear some marker (protein/receptor) that is amenable to targeting. Optimally, the tumor marker protein/receptor is not present on the majority of other cells, such as non-cancer cells or normal cells. Many tumor markers exist and any of these may be suitable for targeting by another drug or therapy administered with an anti-NELL2 antibody as disclosed herein. Common tumor markers include, for example, CD20, carcinoembryonic antigen (CEA), tyrosinase (p97), gp68, TAG-72, HMFG, Sialyl Lewis Antigen, MucA, MucB, PLAP, laminin receptor, erbB, and p155. An alternative aspect of immunotherapy is to combine anticancer effects with immune stimulatory effects. Immune stimulating molecules also exist and include cytokines, such as IL-2, IL-4, IL-12, GM-CSF, gamma-IFN; chemokines, such as MIP-1, MCP-1, IL-8; and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui et al., 1998, Infection Immun., 66(11):5329-5336; Christodoulides et al., 1998, Microbiology, 144(Pt 11):3027-3037); cytokine therapy, e.g., α, β, and γ interferons; IL-1, GM-CSF, and TNF (Bukowski et al., 1998, Clinical Cancer Res., 4(10):2337-2347: Davidson et al., 1998, J. Immunother., 21(5):389-398; Hellstrand et al., 1998, Acta Oncologica, 37(4):347-353); gene therapy, e.g., TNF, IL-1, IL-2, and p53 (Qin et al., 1998, Proc. Natl. Acad. Sci. USA, 95(24): 14411-14416; Austin-Ward and Villaseca, 1998, Revista Medica de Chile, 126(7): 838-845: U.S. Pat. Nos. 5,830,880 and 5,846,945); and monoclonal antibodies, e.g., anti-CD20, anti-ganglioside GM2, and anti-p185 (Hollander, 2012, Front. Immun., 3:3; Hanibuchi et al., 1998, Int. J. Cancer, 78(4): 480-485: U.S. Pat. No. 5,824,311). It is contemplated that one or more anti-cancer therapies may be employed with the antibody therapies described herein.

Surgery. Approximately 60% of individuals with cancer undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as anti-NELL2 antibody treatment as described herein, chemotherapy, radiotherapy, hormonal therapy, gene therapy, immunotherapy, and/or alternative therapies, as well as combinations thereof. Tumor resection refers to physical removal of at least part of a tumor. In addition to tumor resection, treatment by surgery includes laser surgery, cryosurgery, electrosurgery, and microscopically-controlled surgery (Mohs surgery). Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

Other Agents. It is contemplated that other agents may be used in combination with certain aspects of the compositions and methods disclosed herein to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions may increase the anti-hyperproliferative effects on the neighboring hyperproliferative cell population. In some aspects, cytostatic or differentiation agents may be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

Protein Purification. Protein, including antibody and, particularly, anti-NELL2 antibody, purification techniques are well known to those of skill in the art. These techniques involve, at one level, the homogenization and crude fractionation of the cells, tissue, or organ into polypeptide and non-polypeptide fractions. The protein or polypeptide of interest may be further purified using chromatographic and electrophoretic techniques to achieve partial or complete purification (or purification to homogeneity) unless otherwise specified. Analytical methods particularly suited to the preparation of a pure protein or peptide are ion-exchange chromatography, size-exclusion chromatography, reverse phase chromatography, hydroxyapatite chromatography, polyacrylamide gel electrophoresis, affinity chromatography, immunoaffinity chromatography, and isoelectric focusing. A particularly efficient method of purifying peptides is fast-performance liquid chromatography (FPLC) or even high-performance liquid chromatography (HPLC). As is generally known in the art, the order of conducting the various purification steps may be changed, and/or certain steps may be omitted, and still result in a suitable method for the preparation of a substantially purified polypeptide.

A purified polypeptide, such as an anti-NELL2 antibody as described herein, refers to a polypeptide which is isolatable or isolated from other components and purified to any degree relative to its naturally-obtainable state. An isolated or purified polypeptide, therefore, also refers to a polypeptide free from the environment in which it may naturally occur, e.g., cells, tissues, organs, biological samples, and the like. Generally, “purified” will refer to a polypeptide composition that has been subjected to fractionation to remove various other components, and which composition substantially retains its expressed biological activity. A “substantially purified” composition refers to one in which the polypeptide forms the major component of the composition, and as such, constitutes about 50%, about 60%, about 70%, about 80%, about 90%, about 95%, or more of the protein component of the composition.

Various methods for quantifying the degree of purification of polypeptides, such as antibody proteins, are known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity therein, assessed by a “fold purification number.” The actual units used to represent the amount of activity will, of course, be dependent upon the particular assay technique chosen to follow the purification, and whether or not the expressed polypeptide exhibits a detectable activity.

There is no general requirement that the polypeptide will always be provided in its most purified state. Indeed, it is contemplated that less substantially purified products may have utility in some aspects. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “fold” purification than the same technique utilizing a low pressure chromatography system. Methods exhibiting a lower degree of relative purification may have advantages in total recovery of protein product, or in maintaining the activity of an expressed protein.

Affinity chromatography is a chromatographic procedure that relies on the specific affinity between a substance (protein) to be isolated and a molecule to which it can specifically bind, e.g., a receptor-ligand type of interaction. The column material (resin) is synthesized by covalently coupling one of the binding partners to an insoluble matrix. The column material is then able to specifically adsorb the substance from the solution that is passed over the column resin. Elution occurs by changing the conditions to those in which binding will be disrupted/will not occur (e.g., altered pH, ionic strength, temperature, etc.). The matrix should be a substance that does not adsorb molecules to any significant extent and that has a broad range of chemical, physical, and thermal stability. The ligand should be coupled in such a way as to not affect its binding properties. The ligand should also provide relatively tight binding; however, elution of the bound substance should occur without destroying the sample protein desired or the ligand.

Size-exclusion chromatography (SEC) is a chromatographic method in which molecules in solution are separated based on their size, or in more technical terms, their hydrodynamic volume. It is usually applied to large molecules or macromolecular complexes, such as proteins and industrial polymers. Typically, when an aqueous solution is used to transport the sample through the column, the technique is known as gel filtration chromatography, versus the name gel permeation chromatography, which is used when an organic solvent is used as a mobile phase. The underlying principle of SEC is that particles of different sizes will elute (filter) through a stationary phase at different rates, resulting in the separation of a solution of particles based on size. Provided that all of the particles are loaded simultaneously or near simultaneously, particles of the same size should elute together.

High-performance (aka high-pressure) liquid chromatography (HPLC) is a form of column chromatography used frequently in biochemistry and analytical chemistry to separate, identify, and quantify compounds. HPLC utilizes a column that holds chromatographic packing material (stationary phase), a pump that moves the mobile phase(s) through the column, and a detector that shows the retention times of the molecules. Retention time varies depending on the interactions between the stationary phase, the molecules being analyzed, and the solvent(s) used

Pharmaceutical Preparations. Where clinical application of a pharmaceutical composition comprising an anti-NELL2 antibody is undertaken, it is generally beneficial to prepare a pharmaceutical or therapeutic composition appropriate for the intended application. In general, pharmaceutical compositions can comprise an effective amount of one or more polypeptides or additional agents dissolved or dispersed in a pharmaceutically acceptable carrier. In some aspects, pharmaceutical compositions may comprise, for example, at least about 0.1% of a polypeptide or antibody. In some aspects, a polypeptide or antibody may comprise between about 2% to about 75% of the weight of the unit, or between about 25% to about 60%, for example, and any range derivable there between, including the upper and lower values. The amount of active compound(s) in each therapeutically useful composition may be prepared in such a way that a suitable dosage will be obtained in any given unit dose. Factors, such as solubility, bioavailability, biological half-life, route of administration, product shelf life, as well as other pharmacological considerations, are contemplated by one skilled in the art of preparing such pharmaceutical formulations, and as such, a variety of dosages and treatment regimens may be desirable.

Further in some aspects, the composition suitable for administration can be provided in a pharmaceutically acceptable carrier with or without an inert diluent. The carrier should be assimilable and include liquid, semi-solid, e.g., gels or pastes, or solid carriers. Examples of carriers or diluents include but are not limited to fats, oils, water, saline solutions, lipids, liposomes, resins, binders, fillers, and the like, or combinations thereof. As used herein, “pharmaceutically acceptable carrier” includes any and all aqueous solvents (e.g., water, alcoholic/aqueous solutions, ethanol, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings (e.g., lecithin), surfactants, antioxidants, preservatives (e.g., antibacterial or antifungal agents, anti-oxidants, chelating agents, inert gases, parabens (e.g., methylparabens, propylparabens), chlorobutanol, phenol, sorbic acid, thimerosal), isotonic agents (e.g., sugars, sodium chloride), absorption delaying agents (e.g., aluminum monostearate, gelatin), salts, drugs, drug stabilizers (e.g., buffers, amino acids, such as glycine and lysine, carbohydrates, such as dextrose, mannose, galactose, fructose, lactose, sucrose, maltose, sorbitol, mannitol, etc.), gels, binders, excipients, disintegration agents, lubricants, sweetening agents, flavoring agents, dyes, fluid and nutrient replenishers, such like materials and combinations thereof, as would be known to one of ordinary skill in the art. Except insofar as any conventional media, agent, diluent, or carrier is detrimental to the recipient or to the therapeutic effectiveness of the composition contained therein, its use in an administrable composition for the practice of the methods is appropriate. The pH and exact concentration of the various components in a pharmaceutical composition are adjusted according to well-known parameters. In some aspects, the composition can be combined with the carrier in any convenient and practical manner, i.e., by solution, suspension, emulsification, admixture, encapsulation, absorption, grinding, and the like. Such procedures are routine for those skilled in the art.

In some aspects, the compositions may comprise different types of carriers depending on whether they are to be administered in solid, liquid, or aerosol form, and whether it needs to be sterile for the route of administration, such as injection. The compositions can be formulated for administration intravenously, intradermally, transdermally, intrathecally, intra-arterially, intraperitoneally, intranasally, intravaginally, intrarectally, intramuscularly, subcutaneously, mucosally, orally, topically, locally, by inhalation (e.g., aerosol inhalation), by injection, by infusion, by continuous infusion, by localized perfusion bathing target cells directly, via a catheter, via a lavage, in lipid compositions (e.g., liposomes), or by other methods or any combination of the forgoing as would be known to one of ordinary skill in the art. See, for example, Remington's Pharmaceutical Sciences, 18th Ed., 1990. Typically, such compositions can be prepared as either liquid solutions or suspensions; solid or reconstitutable forms suitable for use to prepare solutions or suspensions upon the addition of a liquid prior to injection can also be prepared; and, the preparations can also be emulsified.

The antibodies may be formulated into a composition in a free base, neutral, or salt form. Pharmaceutically acceptable salts include the acid addition salts, e.g., those formed with the free amino groups of a proteinaceous composition, or which are formed with inorganic acids, such as, for example, hydrochloric or phosphoric acids, or such organic acids as acetic, oxalic, tartaric, or mandelic acid. Salts formed with the free carboxyl groups may also be derived from inorganic bases, such as, for example, sodium, potassium, ammonium, calcium, or ferric hydroxides; or such organic bases as isopropylamine, trimethylamine, histidine, or procaine.

In some aspects, a pharmaceutical lipid vehicle composition that includes polypeptides, one or more lipids, and an aqueous solvent may be used. As used herein, the term “lipid” refers to any of a broad range of substances that are characteristically insoluble in water and extractable with an organic solvent. This broad class of compounds is well known to those of skill in the art, and as the term “lipid” is used herein, it is not limited to any particular structure. Examples include compounds that contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether- and ester-linked fatty acids, polymerizable lipids, and combinations thereof. Of course, compounds other than those specifically described herein that are understood by one of skill in the art as lipids are also encompassed by the compositions and methods. One of ordinary skill in the art would be familiar with the range of techniques that can be employed for dispersing a composition in a lipid vehicle. For example, the antibody may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The term “unit dose” or “dosage” refers to physically discrete units suitable for use in a subject, each unit containing a predetermined quantity of the therapeutic antibody or composition containing the therapeutic antibody calculated to produce the desired responses discussed above in association with its administration, i.e., the appropriate route and treatment regimen. The quantity to be administered, both according to number of treatments and unit dose, depends on the effect desired. The actual dosage amount of a composition as described herein that can be administered to a patient or subject can be determined by physical and physiological factors, such as body weight, the age, health, and sex of the subject, the type of disease being treated, the extent of disease penetration, previous or concurrent therapeutic interventions, idiopathy of the subject, the route of administration, and the potency, stability, and toxicity of the particular therapeutic substance. In other non-limiting examples, a dose may also comprise from about 1 microgram/kg/body weight, about 5 microgram/kg/body weight, about 10 microgram/kg/body weight, about 50) microgram/kg/body weight, about 100 microgram/kg/body weight, about 200 microgram/kg/body weight, about 350) microgram/kg/body weight, about 500) microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350) milligram/kg/body weight, about 500 milligram/kg/body weight, to about 1000 milligram/kg/body weight or more per administration, and any range derivable therein. In non-limiting examples of a derivable range from the numbers listed herein, a range of about 5 milligram/kg/body weight to about 100 milligram/kg/body weight, about 5 microgram/kg/body weight to about 500 milligram/kg/body weight, etc., can be administered, based on the numbers described above. The foregoing doses include amounts between those indicated and are intended to also include the lower and upper values of the ranges. The practitioner responsible for administration will, in any event, determine the concentration of active ingredient(s) in a composition and appropriate dose(s) for the individual subject.

The particular nature of the therapeutic composition or preparation is not intended to be limiting. For example, suitable compositions may be provided in formulations together with physiologically tolerable liquid, gel, or solid carriers, diluents, and excipients. In some aspects, the therapeutic preparations may be administered to mammals for veterinary use, such as with domestic animals, and clinical use in humans in a manner similar to other therapeutic agents. In general, the dosage required for therapeutic efficacy will vary according to the type of use and mode of administration, as well as the particularized requirements of individual subjects, as described supra.

NELL2 as a biomarker. Disclosed herein are methods comprising the use of at least one anti-NELL2 antibody as described herein. In some aspects, the methods can comprise detecting the amount of level of NELL2 gene or protein in a subject or in a sample obtained from a subject who has a cancer or tumor or is exhibiting one or more symptoms of cancer. Such methods may be useful in biomarker evaluations of the level of NELL2 in a sample obtained from a subject who has a cancer or tumor or is exhibiting one or more symptoms of a cancer. For example, if the subject's sample is tested and determined to comprise a higher level of NELL2 compared to a reference sample, then the subject is a candidate for treatment with an anti-NELL2 antibody as described herein, alone, or in combination with another agent, for example, would benefit from the treatment. Such methods comprise obtaining a sample from a subject having a cancer or tumor (or exhibiting one or more symptoms of a cancer), testing the sample for the presence of NELL2 derived from the subject's sample using binding methods known and used in the art and as described herein, for example, using an anti-NELL2 antibody as described herein, and administering to the subject an effective amount of an anti-NELL2 antibody alone, or in combination with another agent, if the subject's sample is found to have a higher level of NELL2 when compared to a reference sample. Diagnosing the subject as having a cancer or tumor prior to treatment allows for more effective treatment and benefit to the subject, as the administered anti-NELL2 antibody is more likely to block or inhibit the interaction of the subject's NELL2 with the subject's Robo3, thereby reducing NELL2 levels. In some aspects, the methods can involve first selecting a subject whose cancer or tumor or disease state or condition may be amenable to testing for the presence of NELL2 levels.

Similar methods may be used to monitor the presence of NELL2 levels during a course of treatment or therapy, including combination treatments with an anti-NELL2 antibody and another anticancer drug or treatment, over time, as well as after treatment has ceased. Such methods may also be used in companion diagnostic methods in which a treatment regimen or combination treatment, involves testing or assaying a sample obtained from the subject for NELL2 levels, prior to treatment and during the course of treatment, e.g., monitoring, to determine a successful outcome or the likelihood thereof.

Disclosed herein are methods of detecting increased levels of NELL2 in a biological sample. In some aspects, the methods can comprise: contacting a biological sample with any of the antibodies disclosed herein, detecting of binding of the antibody to NELL2 in the biological sample, and comparing the binding of the antibody to NELL2 in the biological sample to a reference sample, wherein the increased levels of NELL2 in the biological sample indicates cancer. In some aspects, the sample can be a cell sample. In some aspects, the cell sample can be a cancer or a tumor of a subject.

### Fusions and Conjugates

The anti-NELL2 antibodies or polypeptides disclosed herein can also be expressed as fusion proteins with other proteins or chemically conjugated to another moiety. In some aspects, the antibodies or polypeptides can have an Fc portion that can be varied by isotype or subclass, can be a chimeric or hybrid, and/or can be modified, for example to improve effector functions, control half-life or tissue accessibility, augment biophysical characteristics, such as stability, and improve efficiency of production, which can be associated with cost reductions. Many modifications useful in the construction of fusion proteins and methods for making them are known in the art, for example, as reported by Mueller, J. P. et al., 1997, Mol. Immun. 34(6): 441-452; Swann, P. G., 2008, Curr. Opin. Immunol., 20:493-499; and Presta, L. G., 2008, Curr. Opin. Immunol., 20:460-470. In some aspects, the Fc region can be the native IgG1, IgG2, or IgG4 Fc region of the antibody. In some aspects, the Fc region can be a hybrid, for example, a chimera containing IgG2/IgG4 Fc constant regions. Modifications to the Fc region include, but are not limited to, IgG4 modified to prevent binding to Fc gamma receptors and complement: IgG1 modified to improve binding to one or more Fc gamma receptors: IgG1 modified to minimize effector function (amino acid changes); and IgG1 with altered pH-dependent binding to FcRn. The Fc region can include the entire hinge region, or less than the entire hinge region of the antibody.

In some aspects, IgG2-4 hybrids and IgG4 mutants have reduced binding to FcR which can increase their half-life. Representative IG2-4 hybrids and IgG4 mutants are described, for example, in Angal et al., 1993, Molec. Immunol., 30(1): 105-108; Mueller et al., 1997, Mol. Immun., 34(6): 441-452; and U.S. Pat. No. 6,982,323: all of which are hereby incorporated by references in their entireties. In some aspects, the IgG1 and/or IgG2 domain can be deleted. For example, Angal et al., Id., describe proteins in which IgG1 and IgG2 domains have serine 241 replaced with a proline. In some aspects, fusion proteins or polypeptides having at least 10, at least 20, at least 30, at least 40, at least 50, at least 60, at least 70, at least 80, at least 90 or at least 100 amino acids are contemplated.

In some aspects, anti NELL2 antibodies or polypeptides can be linked to or covalently bind or form a complex with at least one moiety. Such a moiety may be, but is not limited to, one that increases the efficacy of the antibody as a diagnostic or a therapeutic agent. In some aspects, the moiety can be an imaging agent, a toxin, a therapeutic enzyme, an antibiotic, a radio-labeled nucleotide, a chemotherapeutic agent, and the like.

In some aspects, antibodies and polypeptides as described herein may be conjugated to a marker, such as a peptide, to facilitate purification. In some aspects, the marker can be a hexa-histidine peptide, i.e., the hemagglutinin “HA” tag, which corresponds to an epitope derived from the influenza hemagglutinin protein (Wilson, I. A. et al., Cell, 37:767-778 (1984)), or the “flag” tag (Knappik, A. et al., Biotechniques 17(4): 754-761 (1994)).

In some aspects, the moiety conjugated to the antibodies and polypeptides as described herein can be an imaging agent that can be detected in an assay. Such imaging agents can be enzymes, prosthetic groups, radiolabels, nonradioactive paramagnetic metal ions, haptens, fluorescent labels, phosphorescent molecules, chemiluminescent molecules, chromophores, luminescent molecules, bioluminescent molecules, photoaffinity molecules, or colored particles or ligands, such as biotin. In some aspects, suitable enzymes include, but are not limited to, horseradish peroxidase, alkaline phosphatase, beta-galactosidase, or acetylcholinesterase; prosthetic group complexes include, but are not limited to, streptavidin/biotin and avidin/biotin; fluorescent materials include, but are not limited to, umbelliferone, fluorescein, fluorescein isothiocyanate, rhodamine, dichlorotriazinylamine fluorescein, dansylchloride or phycoerythrin; luminescent materials include, but are not limited to, luminol; bioluminescent materials include, but are not limited to, luciferase, luciferin, and aequorin; radioactive materials include, but are not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions.

The imaging agent can be conjugated to the antibodies or polypeptides described herein either directly or indirectly through an intermediate (such as, for example, a linker known in the art) using techniques known in the art. See, for example, U.S. Pat. No. 4,741,900 which reports on metal ions that can be conjugated to antibodies and other molecules as described herein for use as diagnostics. Some conjugation methods involve the use of a metal chelate complex employing, for example, an organic chelating agent, such as diethylenetriaminepentaacetic acid anhydride (DTPA); ethylenetriaminetetraacetic acid; N-chloro-p-toluenesulfonamide; and/or tetrachloro-3-6a-diphenylglycouril-3, attached to the antibody. Monoclonal antibodies can also be reacted with an enzyme in the presence of a coupling agent such as glutaraldehyde or periodate. Conjugates with fluorescein markers can be prepared in the presence of these coupling agents or by reaction with an isothiocyanate.

In some aspects, the anti-NELL2 antibodies polypeptides as described herein can be conjugated to a second antibody to form an antibody heteroconjugate, for example, as described in U.S. Pat. No. 4,676,980. Such heteroconjugate antibodies can additionally bind to haptens (e.g., fluorescein), or to cellular markers.

In some aspects, the anti-NELL2 antibodies or polypeptides described herein can also be attached to solid supports, which can be useful for carrying out immunoassays or purification of the target antigen or of other molecules that are capable of binding to the target antigen that has been immobilized to the support via binding to an antibody or antigen binding fragment as described herein. Such solid supports include, but are not limited to, glass, cellulose, polyacrylamide, nylon, polystyrene, polyvinyl chloride or polypropylene.

### Kits and Diagnostics

Disclosed herein are kits comprising therapeutic agents and/or other therapeutic and delivery agents disclosed herein. Disclosed herein are kits comprising the polypeptides, antibodies or fusion constructs disclosed herein. In some aspects, the kits can be used for preparing and/or administering a therapy involving the polypeptides, antibodies or fusion constructs described herein. In some aspects, the kits can be used for preparing and/or administering a therapy involving the anti-NELL2 antibodies described herein. The kits can comprise one or more sealed vials containing any of the pharmaceutical compositions as described herein. The kits can include, for example, at least one NELL2 antibody, as well as reagents to prepare, formulate, and/or administer one or more anti-NELL2 antibodies or to perform one or more steps of the described methods. In some aspects, the kits can also comprise a suitable container means, which is a container that will not react with components of the kit, such as an Eppendorf tube, an assay plate, a syringe, a bottle, or a tube. The container may be made from sterilizable materials, such as plastic or glass.

The kits can further include an instruction sheet that outlines the procedural steps of the methods set forth herein, and will follow substantially the same procedures as described herein or are known to those of ordinary skill. The instruction information may be in a computer readable medium containing machine-readable instructions that, when executed using a computer, cause the display of a real or virtual procedure of delivering a pharmaceutically effective amount of the therapeutic agent.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, certain changes and modifications may be practiced within the scope of the appended claims.

### EXAMPLES

### Example 1: NELL2-Cdc42 Signaling Regulates BAF Complexes and Ewing Sarcoma Cell Growth

Described herein is a NELL2 cytokine signaling pathway that regulates the BAF complexes and Ewing sarcoma growth. The results demonstrate that NELL2 signaling inhibits cdc42, which induces actin polymerization and BAF complex disassembly.

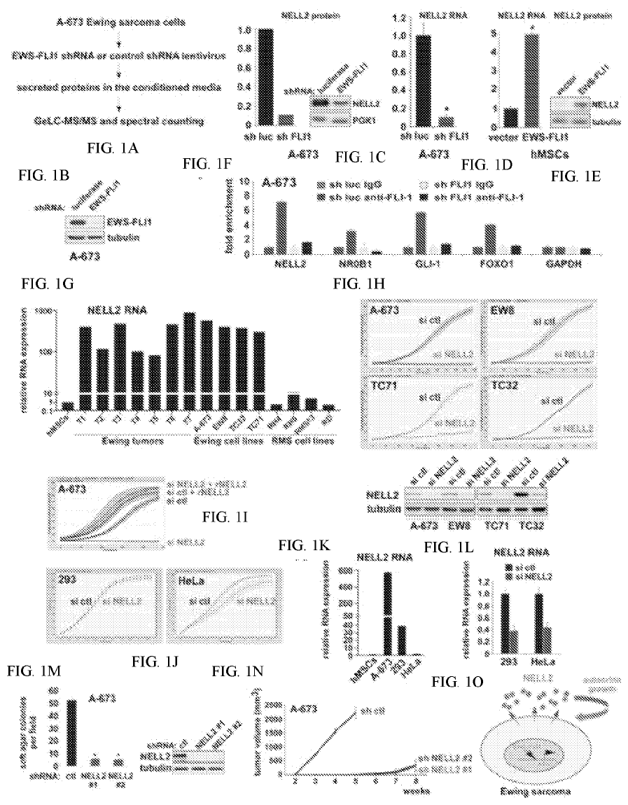

Results. Secretome proteomics identifies NELL2 as a target of EWS-FLI1. While the gene expression program regulated by EWS-FLI1 has been well studied, the effect of EWS-FLI1 on protein secretion has not been systematically analyzed. shRNA-mediated silencing of EWS-FLI1 and secretome proteomics was used to address this question (FIG. 1A). A-673 Ewing sarcoma cells were infected with lentiviruses expressing an shRNA against FLI1 C-terminal region or luciferase (control). After verifying the silencing of EWS-FLI1 protein (FIG. 1B), the proteins secreted in the conditioned medium were identified by mass spectrometry and quantified by spectral counting.

One of the high-confidence proteins that displayed a significant alteration in abundance upon EWS-FLI1 silencing was NELL2, which exhibited a nearly 10-fold decrease after silencing EWS-FLI1 (FIG. 1C). NELL2 is predominantly expressed in neural tissues and has been studied as an axon guidance molecule (Jaworski et al., 2015; Jiang et al., 2009; and Nakamura et al., 2015).

It was found that NELL2 transcript levels are significantly reduced upon EWS-FLI1 silencing (FIG. 1D). Conversely, lentiviral expression of EWS-FLI1 in human mesenchymal stem cells, putative cells of origin of Ewing sarcoma, increased NELL2 transcript and protein levels (FIG. 1E). Using chromatin immunoprecipitation, it was determined that endogenous EWS-FLI1 binds to the NELL2 gene promoter in A-673 cells and this binding was abolished by EWS-FLI1 silencing (FIG. 1F), suggesting that NELL2 is a direct transcriptional target of EWS-FLI1. Consistent with NELL2 being an EWS-FLI1 target, NELL2 is highly expressed in Ewing sarcoma tumors and cell lines compared with mesenchymal stem cells (FIG. 1G).

Ewing sarcoma is dependent on NELL2. To elucidate the role of NELL2 in Ewing sarcoma, the effects of manipulation of NELL2 expression was tested. Silencing of NELL2 by a pool of siRNAs strongly inhibited the proliferation of the fifteen Ewing sarcoma cell lines tested (A-673, EW8, TC71, TC32, SK-N-MC, CHLA-9, ES1, ES2, ES3, ES6, ES7, ES8, SK-NEP-1, SK-ES-1, and RD-ES; FIG. 1H). Importantly, the proliferation inhibition by NELL2 silencing was completely rescued by the addition of purified recombinant NELL2 protein to the culture medium (FIG. 1I), indicating that Ewing sarcoma is dependent on extracellular NELL2. NELL2 siRNAs had little effect on the proliferation of 293/HEK293 and HeLa cells (FIG. 1J). NELL2 RNA expression was lower in 293 and HeLa cells than in A-673 cells (FIG. 1K) and NELL2 siRNAs did reduce NELL2 RNA levels in 293 and HeLa cells (FIG. 1L).

Lentivirus-mediated shRNA expression was used to stably silence NELL2. shRNA-mediated silencing of NELL2 severely impaired the anchorage-independent growth (FIG. 1M) and xenograft tumorigenicity (FIG. 1N) of Ewing sarcoma cells. These results suggest that Ewing sarcoma is dependent on NELL2, a newly identified EWS-FLI1 target (FIG. 1O).

Robo3 serves as the NELL2 receptor in Ewing sarcoma. NELL2 was previously identified as the ligand for the axon guidance receptor, Robo3 (Jaworski, A., et al. (2015). Science 350, 961-965). NELL2 repels axons through Robo3 and contributes to commissural axon guidance to the midline Jaworski, A., et al. (2015). Science 350, 961-965). Additional experiments were carried out to determine whether Robo3 also serves as the NELL2 receptor in Ewing sarcoma.

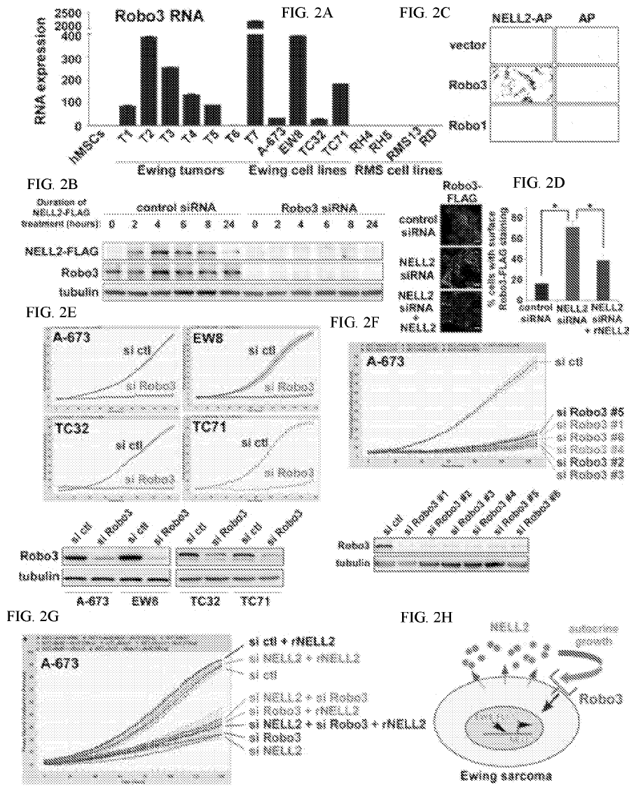

Robo3 expression was readily detectable in Ewing sarcoma tumors and cell lines, but undetectable in mesenchymal stem cells (FIG. 2A). The role of Robo3 as the NELL2 receptor in Ewing sarcoma was tested using ligand-binding assays (FIG. 2B). C-terminally FLAG-tagged NELL2 was produced from transfected 293T/HEK293T cells and was incubated with A-673 cells that were transfected with Robo3 siRNA pool or control siRNA pool. After washing the cells to remove unbound NELL2-FLAG, a whole cell lysate was prepared and analyzed by immunoblotting to assess the binding of NELL2-FLAG to A-673 cells. As shown in FIG. 2B, NELL2-FLAG bound to control siRNA transfected A-673 cells, and this binding was abolished by silencing of Robo3, indicating that NELL2 binds Robo3 in Ewing sarcoma. The specificity of the NELL2-Robo3 interaction was further confirmed by ligand-binding assays using alkaline phosphatase-fused NELL2 (NELL2-AP) (FIG. 2C). NELL2-AP bound to COS cells expressing Robo3, but not Robo1 or vector (FIG. 2C). NELL2 silencing resulted in accumulation of Robo3-FLAG on the A-673 cell surface, which was reversed by the addition of recombinant NELL2 (FIG. 2D).

Next, the role of Robo3 was examined in Ewing sarcoma by siRNA-mediated silencing. Robo3 silencing by siRNA pool strongly inhibited the proliferation of Ewing sarcoma cell lines (FIG. 2E). This was further confirmed by six individual siRNAs targeting different regions of Robo3 (FIG. 2F), which each reduced Robo3 protein levels and inhibited cell proliferation. Subsequently, it was tested whether NELL2 requires Robo3 to stimulate Ewing sarcoma cell proliferation. Recombinant NELL2 was able to rescue the proliferation of cells transfected with NELL2 siRNAs, but not cells that were co-transfected with NELL2 siRNAs and Robo3 siRNAs (FIG. 2G), demonstrating that NELL2 requires Robo3 to transmit growth signaling. These results indicate that Robo3 functions as the NELL2 receptor in Ewing sarcoma (FIG. 2H).

NELL2 signaling downregulates cdc42. In Slit-Robo1/2 signaling, the binding of Slit ligand to Robo1/2 receptor activates Slit-Robo GTPase activating proteins (srGAPs), which inactivate Rho family G proteins, primarily cdc42, resulting in axon repulsion (Wong. K., et al. (2001). Cell 107, 209-221). Unlike Robo1/2, mammalian Robo3 does not bind Slit and, instead, uses NELL2 as the ligand (Jaworski, A., et al. (2015). Science 350, 961-965). While the signaling downstream of the NELL2-Robo3 interaction has not been studied, the binding site for srGAPs, conserved cytoplasmic motif 3 (Wong, K., et al. (2001). Cell 107, 209-221, is also present in Robo3, which led to testing whether NELL2-Robo3 signaling inhibits cdc42 through srGAPs.

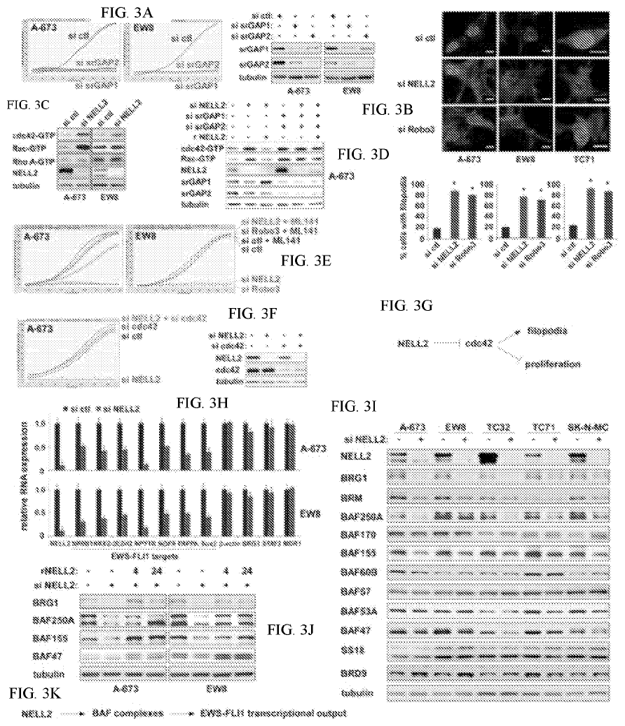

It was found that the silencing of srGAP1/2 strongly inhibits the proliferation of Ewing sarcoma cells (FIG. 3A). It is noted the cross-silencing of srGAP1 and srGAP2 (FIG. 3A, right), which might reflect their hetero-oligomerization (Muller, P. M., et al. (2020) Nat Cell Biol 22, 498-511). Silencing of either NELL2 or Robo3 in Ewing sarcoma cells resulted in increased filopodia (FIG. 3B), which is primarily regulated by cdc42 (Mattila, P. K., and Lappalainen, P. (2008). Nat Rev Mol Cell Biol 9, 446-454). NELL2 silencing robustly increases the levels of active cdc42 and active Rac in Ewing sarcoma cells Using the pull-down of cell lysate with GST-PAKI, which selectively interacts with GTP-bound active cdc42 and active Rac, we showed that (FIG. 3C). Activation of cdc42 and Rac by NELL2 silencing was abolished by the addition of recombinant NELL2 (FIG. 3D, lane 1-3). The silencing of srGAP1 and srGAP2 increased the levels of active cdc42 and active Rac, which were not affected by NELL2 silencing or recombinant NELL2 (FIG. 3D, lane 4-6). These results indicate that srGAPs act downstream of NELL2 to inhibit cdc42 and Rac. Furthermore, proliferation inhibition of Ewing sarcoma cells induced by silencing of either NELL2 or Robo3 was abolished by a cdc42-specific inhibitor, ML141 (FIG. 3E), which does not inhibit Rac. Ras, or Rab (Surviladze, Z., et al. (2010) A Potent and Selective Inhibitor of Cdc42 GTPase. In Probe Reports from the NIH Molecular Libraries Program (Bethesda (MD)). Additionally, siRNA-mediated silencing of cdc42 abrogated the proliferation inhibition by NELL2 silencing (FIG. 3F). These results suggest that NELL2 signaling primarily inhibits cdc42, the normal function of which is to stimulate filopodia formation and inhibit the proliferation of Ewing sarcoma cells (FIG. 3G).

NELL2 signaling upregulates the BAF chromatin remodeling complexes and EWS-FLI1 transcriptional output. Analysis of gene expression changes induced by NELL2 silencing revealed reduced expression of EWS-FLI1 target genes in Ewing sarcoma (FIG. 3H). As EWS-FLI1 was recently shown to recruit the BAF chromatin remodeling complexes to activate its target genes (Boulay, G., et al. (2017) Cell 171, 1-16), the effect of NELL2 silencing on the levels of the BAF complex subunits in five Ewing sarcoma cell lines were examined and the results show that NELL2 silencing selectively reduces BRG1, BRM, BAF250A/ARID1A, BAF155, and BAF47 (FIG. 3I). In addition, a few other subunits such as BAF53A and BRD9 were affected by NELL2 silencing in a cell line-specific manner. NELL2 silencing reduced the protein levels of BRG1, BAF155, and BAF47 (FIG. 3I), but not their RNA levels (FIG. 3H). This suggests that NELL2-cdc42 signaling regulates the protein stability of these BAF subunits, which was confirmed.

Importantly, the reduced BAF subunit levels in NELL2 siRNA-transfected cells were rescued by the addition of recombinant NELL2 to the culture medium (FIG. 3J). These results suggest that extracellular NELL2 signals to increase the protein levels of important BAF subunits and enhances the transcriptional output of EWS-FLI1 (FIG. 3K).

cdc42 downregulates the BAF complexes. Because NELL2 signaling affects both cdc42 and BAF complexes, the link between cdc42 and BAF was next investigated. While NELL2 silencing normally results in reduced BRG1, BAF250A, BAF155, and BAF47 in Ewing sarcoma cells, the cdc42 inhibitor, ML141, abolished this response, suggesting that cdc42 acts downstream of NELL2 to regulate the BAF complexes. The role of cdc42 in BAF downregulation was further supported by activating cdc42. A four-hour treatment with a cdc42/Rac/Rho A activator, CN04, significantly reduced the levels of BRG1, BAF250A. BAF155, and BAF47 in A-673 cells. Furthermore, transfection of a constitutively active cdc42 Q61L mutant reduced BRG1, BRM, BAF250A, BAF155, and BAF47 in A-673 cells. Reduced protein levels of BRG1, BAF250A, BAF155, and BAF47 upon CN04 treatment were restored by a proteasome inhibitor, MG-132, suggesting that CN04 induces the proteasomal degradation of these BAF subunits. Importantly, the BAF downregulation by cdc42 is not limited to Ewing sarcoma cells. CN04 treatment reduced BRG1, BAF155, and BAF47, but not BAF60B, in 293 (embryonic kidney), HeLa (cervical carcinoma), HCT116 (colon adenocarcinoma), and IMR-90) (fibroblast) cells. CN04 also reduced BRG1 and BAF155 in BT-12 malignant rhabdoid tumor cells, which lack BAF47 expression due to inactivating mutations, suggesting that BAF47 is not required for the downregulation of BRG1 and BAF155 by CN04. By transfecting active and inactive mutants of cdc42, Rac1, and Rho A in 293T cells, it was found that active cdc42 (Q61L), active Rac1 (Q61L), and, to a lesser extent, active Rho A (Q63L) reduce endogenous BRG1, BAF155, and BAF47 or co-transfected GFP-BRG1. These results indicate that cdc42 and other Rho family G-proteins downregulate the BAF complexes.

NELL2-cdc42 signaling regulates actin polymerization and assembly of BAF complexes. The BAF complexes contain B-actin monomer as a subunit, which is bound to the ATPase subunit, BRG1 (Zhao, K., et al. (1998) Cell 95, 625-636). B-actin monomer as well as an actin-related subunit, BAF53, is required for maximal ATPase activity of BRG1 and for association of the BAF complexes with the nuclear matrix (Zhao, K., et al. (1998) Cell 95, 625-636). Because cdc42 is a well-established activator of actin polymerization, the status of actin in the BAF complexes were analyzed upon cdc42 activation.

Anti-BRG1 immunoprecipitation of A-673 cell nuclear extract revealed that CN04 treatment significantly increases the abundance of B-actin and, to a lesser extent, BAF53A in the BAF complexes in A-673 cells. A-673 cells were treated or not with 1 μg/ml CN04 for four hours, and the nuclear extract was immunoprecipitated with anti-BRG1 antibody or control IgG. The abundance of indicated BAF subunits in the BAF complexes was examined by immunoblotting.

Similar results were obtained by immunoprecipitation with anti-BAF57, antibody. The BAF complexes immuno-purified by anti-BRG1 antibody were also analyzed by dot blot for the binding to biotin-conjugated phalloidin, which selectively binds actin polymers. CN04 treatment increased the phalloidin-binding of BAF complexes in A-673 cells. The BAF complexes immuno-purified as in A were analyzed for the binding to Biotin-XX-Phalloidin by dot blot. Similar results were obtained by immuno-purifying the BAF complexes using anti-BAF57 antibody.

FLAG-tagged BRG1 was transfected in 293T cells and the BAF complexes were isolated by anti-FLAG immunoprecipitation of nuclear extract. CN04 treatment increased the abundance of B-actin and BAF53A in the FLAG-BRG1-containing BAF complexes in 293T cells. 293T cells were transfected with FLAG-BRG1 or FLAG-vector and were treated or not with 1 μg/ml CN04 for four hours as indicated. FLAG-BRG1-containing BAF complexes were isolated by anti-FLAG immunoprecipitation and the abundance of indicated BAF subunits was examined by immunoblotting. CN04 treatment increases the phalloidin reactivity of BAF complexes in 293T cells. The phalloidin dot blot analysis revealed that FLAG-BRG1-containing BAF complexes purified from CN04-treated 293T cells react more strongly with phalloidin than those from untreated cells. The BAF complexes immuno-purified as in C were analyzed for the binding to Biotin-XX-Phalloidin by dot blot.

The pull-down of A-673 cell nuclear extract with biotin-conjugated phalloidin revealed that more BRG1 and BRM are pulled down by biotin-conjugated phalloidin upon CN04 treatment (1 μg/ml for four hours), NELL2 silencing, and treatment with Jasplakinolide, a pharmacological inducer of actin polymerization.

Collectively, these results suggest that polymerized actin is bound to the BAF complexes upon cdc42 activation or NELL2 silencing.

Then gel filtration chromatography of A-673 cell nuclear extract was used to evaluate the assembly of the BAF complexes in response to NELL2 signaling. NELL2 signaling reversibly regulates the assembly of BAF complexes in A-673 cells. Nuclear extract of A-673 cells treated as indicated was analyzed by gel filtration chromatography and immunoblotting. In control siRNA-transfected cells, the BAF subunits mostly co-eluted with BRG1 in high molecular weight complexes (fractions 40-52). NELL2 silencing caused a size shift of the BAF complexes to a lower molecular weight range (fractions 52-64). Importantly, four-hour treatment with recombinant NELL2 largely restored the size of the BAF complexes. NELL2 silencing did not affect the elution profiles of Rpb1 and cdc6, a component of RNA polymerase II and replication pre-initiation complex, respectively. These results indicate that extracellular NELL2 signals to regulate the assembly of the BAF complexes.

Gel filtration chromatography was also used to assess the effect of cdc42 activation on the BAF complex assembly. It was found that CN04 treatment reduces the size of BAF complexes in A-673 cells and 293T cells. Furthermore, transfection of cdc42 Q61L active mutant similarly reduced the size of BAF complexes in 293T cells. These results demonstrate that NELL2 silencing or cdc42 activation results in disassembly of the BAF complexes.

SW13 adrenal carcinoma cells do not express BRG1 and BRM (Muchardt, C., and Yaniv, M. (1993) EMBO J 12, 4279-4290); thus actin is not associated with the BAF complexes in this cell line (Zhao, K., et al. (1998) Cell 95, 625-636). Importantly, CN04 treatment barely affected the size of BAF complexes in vector-transfected SW13 cells whereas, in BRG1-transfected SW13 cells, CN04 treatment significantly reduced the size of BAF complexes. Furthermore, CN04 treatment did not affect the protein levels of BAF155 and BAF47 in vector-transfected SW13 cells, but reduced these BAF subunits in BRG1-transfected SW13 cells. These results suggest that cdc42 induces actin polymerization, leading to BAF complex disassembly and degradation of specific BAF subunits.

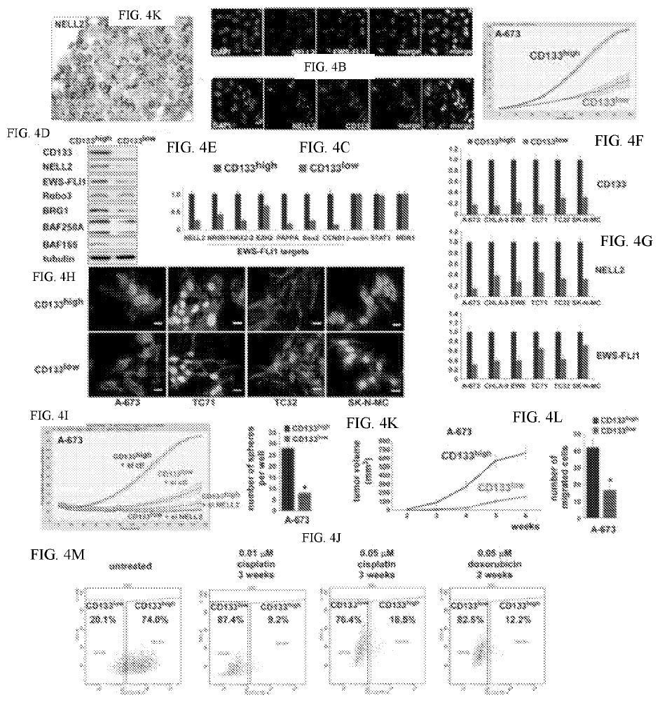

NELL2^{high}CD133^{high} EWS-FL1^{high} and NELL2^{low}CD133^{low}EWS-FLI1^{low} populations in Ewing sarcoma display phenotypes consistent with high and low NELL2 signaling, respectively. Immunohistochemical staining of Ewing sarcoma tumors revealed that NELL2 expression is heterogeneous (FIG. 4A). The heterogeneity of NELL2 expression was also observed in Ewing sarcoma cell lines. Immunofluorescent staining of A-673 cells detected the cells that express NELL2 and the cells that do not detectably express NELL2, and these correlated well with the expression of EWS-FLI1 (FIG. 4B). shRNA-mediated silencing of EWS-FLI1 abolished the staining of EWS-FLI1 in A-673 cells, indicating the specificity of staining. Furthermore, it was found that NELL2 expression also correlates with the expression of CD133 (FIG. 4C), a transmembrane protein used as a marker of stem cells in some normal tissues and cancers (Glumac, P. M., and LeBeau, A. M. (2018) Clinical and translational medicine 7, 18).

Using anti-CD133 (AC133) antibody, Ewing sarcoma cells were sorted into the CD133^{high} and CD133^{low} populations, which revealed that the CD133^{low} population displays lower expression of NELL2, EWS-FLI1, BRG1, BAF250A, and BAF155 than the CD133^{high} population (FIG. 4D, 4G). There was comparable expression of Robo3 in the two populations (FIG. 4D). The CD133^{low} population also exhibited lower expression of EWS-FLI1 target genes (FIG. 4E), slower proliferation (FIG. 4F), and more filopodia (FIG. 4H) than the CD133^{high} population. These results suggest that NELL2 expression is heterogeneous in Ewing sarcoma and that the NELL2^{high}CD133^{high}EWS-FLI1^{high} and NELL2^{low}CD133^{low}EWS-FLI1^{low} populations display phenotypes consistent with high and low NELL2 signaling, respectively.

Although the NELL2^{low}CD133^{low}EWS-FLI1^{low} population expresses lower levels of NELL2 than the NELL2^{high}CD133^{high}EWS-FLI1^{high} population and displays phenotypes consistent with lower NELL2 signaling, NELL2 silencing inhibited the proliferation of both populations (FIG. 4I), indicating that both populations are dependent on NELL2. When the cultures of the CD133^{high} and CD133^{low} populations were continued for a longer time, it was observed that the CD133^{high} population generated the CD133^{low} population and vice versa, demonstrating the interconversion of the two populations. The cells were also dissociated from a patient-derived xenograft (PDX) tumor of Ewing sarcoma (NCH-EWS-1), which was never propagated by in vitro culture, and sorted the cells into the CD133^{high} and CD133^{low} populations, which recapitulated the phenotypes of cell line-derived CD133^{high} and CD133^{low} populations. The purity of tumor cells derived from the PDX tumor were assessed by staining for CD99, a widely used marker for Ewing sarcoma, and it was found that both CD133^{high} and CD133^{low} cells derived from the PDX tumor are predominantly positive for CD99.

The CD133^{high} population exhibited higher tumor sphere formation (FIG. 4J), higher xenograft tumorigenicity (FIG. 4K), and higher migration rate (FIG. 4L) than the CD133^{low} population. Interestingly, however, the CD133^{low} population is more resistant to the chemotherapeutic drugs cisplatin and doxorubicin than the CD133^{high} population. Prolonged treatment with cisplatin or doxorubicin enriched the CD133^{low} population (FIG. 4M), further supporting the chemo-resistance of CD133^{low} cells.

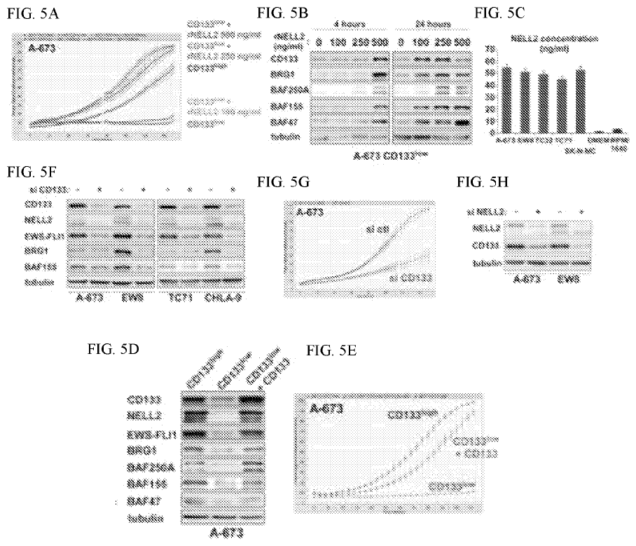

NELL2. CD133, and EWS-FLI1 positively regulate each other and increase the BAF subunits and cell proliferation in Ewing sarcoma. Because the NELL2^{low}CD133^{low}EWS-FLI1^{low} population displays phenotypes consistent with low NELL2 signaling, it was tested whether increasing the levels of NELL2 alters the phenotypes of this population. It was found that the addition of recombinant NELL2 rescues the slow growth of the CD133^{low} population in a dose-dependent manner (FIG. 5A). Furthermore, recombinant NELL2 increased CD133, BRG1, BAF250A, BAF155, and BAF47 in the CD133^{low} population in a dose- and time-dependent fashion (FIG. 5B). While recombinant NELL2 at 250 ng/ml or higher was able to rescue the slow growth of the CD133^{low} population (FIG. 5A), the ELISA measurements determined the NELL2 concentration in the culture supernatant of Ewing sarcoma cell lines to be approximately 50 ng/ml (FIG. 5C), which is insufficient to rescue the slow growth of the CD133^{low} population (FIG. 5A). This suggests that cross-feeding of NELL2 from the CD133^{high} population would not rescue the slow growth of the CD133^{low} population in a mixed culture.

The effect of increasing the levels of CD133 in the CD133^{low} population was also tested. Lentivirus was used to express CD133 in the CD133^{low} population, which increased CD133 to the levels comparable to those of the CD133^{high} population (FIG. 5D). Increasing CD133 in the CD133^{low} population resulted in increased NELL2, EWS-FLI1, BRG1, BAF250A, and BAF155 protein levels (FIG. 5D) as well as increased cell proliferation (FIG. 5E). Conversely, silencing of CD133 in Ewing sarcoma cells resulted in reduced NELL2, EWS-FLI1, BRG1, and BAF155 protein levels (FIG. 5F) and reduced cell proliferation (FIG. 5G). Additionally, silencing of NELL2 resulted in reduced CD133 in Ewing sarcoma cells (FIG. 5H).

Finally, shRNA-mediated silencing of EWS-FLI1 resulted in reduced NELL2 and CD133 in Ewing sarcoma cells (FIG. 5I). Six different promoters have been identified in the upstream region of the human CD133 gene (Sompallae, R., et al. (2013). Frontiers in genetics 4, 209). Chromatin immunoprecipitation assays demonstrated strong binding of EWS-FLI1 to the P2 and P6 promoters of the CD133 gene (FIG. 5J). The silencing of EWS-FLI1 reduced CD133 transcript levels in A-673 cells (FIG. 5K), while exogenous EWS-FLI1 expression induced CD133 transcript levels in human mesenchymal stem cells (FIG. 5L). These results indicate that CD133 is a transcriptional activation target of EWS-FLI1.

Collectively, these findings suggest that NELL2, CD133, and EWS-FLI1 positively regulate each other (FIG. 5M), increasing the BAF subunits and cell proliferation in Ewing sarcoma.

The silencing of NELL2 in A-673 cells abolished the binding of the BAF complexes to the EWS promoter and suppressed the expression of EWS and EWS-FLI1. The silencing of NELL2 in A-673 cells also abolished the binding of EWS-FLI1 and the BAF complexes to the P2 and P6 promoters of CD133 and suppressed CD133 expression.

The binding of the BAF complexes to the EWS promoter was detected in the CD133^{high} population, but not in the CD133^{low} population of A-673 cells. Increasing CD133 in the CD133^{low} population induced the binding of the BAF complexes to the EWS promoter and induced EWS and EWS-FLI1 expression.

The binding of EWS-FLI1 and the BAF complexes to the NELL2 promoter was detected in the CD133^{high} population, but not in the CD133^{low} population of A-673 cells. Increasing CD133 in the CD133^{low} population induced the binding of EWS-FLI1 and the BAF complexes to the NELL2 promoter and induced NELL2 expression.

As demonstrated earlier, EWS-FLI1 directly activates the transcription of NELL2 (FIGS. 1D-F) and CD133 (FIGS. 5J-L).

These results clarify the mechanistic basis for the mutually enhancing relationship among NELL2, EWS-FLI1, and CD133 as summarized in FIG. 5M.

CD133 signaling downregulates cdc42 and upregulates the BAF complexes. Regarding the signaling downstream of CD133, it was found that the CD133^{low} population displays higher levels of active cdc42 and active Rac than the CD133^{high} population and that increasing CD133 in the CD133^{low} population results in reduced active cdc42 and active Rac, suggesting that CD133 signals to inhibit cdc42 and Rac. The treatment of the CD133^{low} population with the cdc42 inhibitor ML141 increased BRG1, BAF155, and BAF47 protein levels and increased cell proliferation, suggesting that cdc42 mediates CD133 signaling. The C-terminal cytoplasmic tail of CD133 directly binds and activates Src (Liu, C., et al. (2016) J Biol Chem 291, 15540-15550) and it was found that the CD133^{low} population displays reduced active Src (Src Y419 phosphorylation), which was restored by increasing CD133 levels.

One of the well-established substrates of Src is caveolin-1 (Glenney, J. R., Jr., and Zokas, L. (1989) J Cell Biol 108, 2401-2408), a component of plasma membrane invaginations called caveolae. Caveolin-1 is highly expressed in Ewing sarcoma and promotes cell proliferation and tumorigenicity (Tirado, O. M., et al. (2006) Cancer Res 66, 9937-9947). Caveolin-1 functions as an inhibitor (guanine nucleotide dissociation inhibitor) of cdc42 (Nevins, A. K., and Thurmond, D. C. (2006) J Biol Chem 281, 18961-18972) and this activity of caveolin-1 appears to be enhanced when caveolin-1 is tyrosine phosphorylated (Cheng, Z. J., et al. (2010) J Biol Chem 285, 15119-15125). It was found that the CD133^{low} population displays reduced tyrosine phosphorylation of caveolin-1, which was restored by increasing CD133 levels.

Treatment of A-673 cells with a Src inhibitor, dasatinib, resulted in dose-dependent inhibition of Src (Src Y419 phosphorylation), tyrosine de-phosphorylation of caveolin-1, and downregulation of BRG1, BAF155, and BAF47. Dasatinib treatment also increased active cdc42 and active Rac in unsorted A-673 cells and in CD133-overexpressing CD133^{low} population. The latter data suggest that Src inhibition counteracts the downregulation of cdc42/Rac by CD133.

The silencing of caveolin-1 in A-673 cells reduced tyrosine-phosphorylated caveolin-1, BRG1, BAF155, and BAF47. Caveolin-1 silencing also activated cdc42 and Rac in unsorted A-673 cells and re-activated cdc42 and Rac in CD133-overexpressing CD133^{low} population. These results suggest that CD133 downregulates cdc42 through Src and caveolin-1.

It was recently reported that the prodomain of a BMP family cytokine, GDF6, binds CD99 and recruits CSK to the cytoplasmic domain of CD99, leading to inhibition of Src in Ewing sarcoma (Zhou, F., et al. (2020) Cell reports 33, 108332). Thus, the effect of GDF6 silencing on caveolin-1, cdc42, and Rac was tested. It was found that GDF6 silencing results in activation of Src, but does not induce phosphorylation of caveolin-1 or suppression of cdc42 and Rac. CD133 and GDF6-CD99-CSK may target distinct pools of Src, and the former affects caveolin-1 and cdc42/Rac.

Lentiviral expression of CD133 in 293, 293T, HeLa, HCT116, and Aska (synovial sarcoma) cells resulted in increased active Src (Src Y419 phosphorylation), increased tyrosine phosphorylation of caveolin-1, diminished cdc42 and Rac activity, and increased BAF subunits (BRG1, BAF155, and BAF47). This suggests that CD133-BAF signaling also occurs in non-Ewing sarcoma cells.

Because both NELL2 signaling and CD133-Src signaling inhibit cdc42 and upregulate the BAF complexes, it was tested whether NELL2 signaling acts independently of CD133-Src signaling. In the presence of dasatinib, which depleted phosphorylated active Src, the silencing of NELL2 resulted in further reduced levels of BRG1, BAF155, and BAF47, which was rescued by recombinant NELL2 treatment (250 ng/ml). This indicates that, unlike CD133 signaling, NELL2 signaling does not depend on Src activity.

Taken together, these results demonstrate that NELL2 and CD133 downregulate cdc42 and upregulate the BAF complexes by distinct mechanisms.

Discussion NELL2-cdc42 signaling appears to affect the levels of a subset of the BAF subunits, BRG1, BRM, BAF250A, BAF155, and BAF47 (FIG. 3I). Some of these subunits were shown to affect each other's protein levels (siRNA-mediated silencing of one subunit results in reduced protein levels of a few other subunits, but not the remaining subunits) (Watanabe et al., 2014, Cancer Res 74, 2465-2475). In addition, mouse BAF155 (SRG3) was shown to bind and stabilize BRG1, BAF47, and BAF60A by attenuating their proteasomal degradation (Sohn et al., 2007, J Biol Chem 282, 10614-10624). It is noted that the BAF subunits regulated by NELL2-cdc42 signaling or reported to display interdependent protein stability do not necessarily correlate with the proposed BAF complex assembly pathway (Mashtalir et al., 2018, Cell 175, 1272-1288 e1220). It is likely that NELL2-cdc42 signaling influences the level of the module within the BAF complexes which displays interdependent protein stability. Because the NELL2-cdc42-regulated module contains essential catalytic subunits (BRG1 and BRM), BAF47, which is required for BAF-chromatin association (Nakayama et al., 2017, Nat Genet 49, 1613-1623), and BAF250A, which is the most frequently mutated BAF subunit in cancers (Hodges et al., 2016, The Many Roles of BAF (mSWI/SNF) and PBAF Complexes in Cancer. Cold Spring Harbor perspectives in medicine 6; Kadoch and Crabtree, 2015, Science advances/, e1500447; and St Pierre and Kadoch, 2017, Curr Opin Genet Dev 42, 56-67), it was surmised that NELL2 signaling upregulates the activity of the BAF complexes. This is supported by the reduced EWS-FLI1 transcriptional output upon NELL2 silencing in Ewing sarcoma (FIG. 3H).

BAF250A and EWS-FLI1 physically and functionally interact in Ewing sarcoma (Selvanathan et al., 2015, Oncogenic fusion protein EWS-FLI1 is a network hub that regulates alternative splicing. Proc Natl Acad Sci USA; and Selvanathan et al., 2019, Nucleic Acids Res 47, 9619-9636) and these two proteins stabilize each other (Selvanathan et al., 2019, Nucleic Acids Res 47, 9619-9636). Thus it is possible that, in addition to the disassembly and destabilization of the BAF complexes, the destabilization of the EWS-FLI1 protein by reduced BAF250A contributes to the reduced EWS-FLI1 transcriptional output upon NELL2 silencing. The destabilization of EWS-FLI1 by reduced BAF250A may also contribute to the reduced EWS-FLI1 protein levels in the CD133^{low} population. Additionally, it is also possible that NELL2-BAF signaling and other modulators of chromatin remodeling such as RING1B (Sanchez-Molina et al., 2020, Science advances 6.) cooperatively regulate EWS-FLI1 transcriptional output in Ewing sarcoma.

While NELL2 signaling is tumor promoting in Ewing sarcoma where the BAF complexes are utilized by EWS-FLI1 to activate the target genes and are, thus, playing a tumor promoting role, it is possible that NELL2 signaling mediates tumor suppression in other cancers. The BAF complexes have both tumor-promoting and tumor-suppressing roles, depending on the type of cancer. Different BAF subunits are recurrently inactivated by mutations in different cancers, suggesting that the BAF complexes act as tumor suppressors in these cancers. By upregulating the BAF complexes, NELL2 signaling can also mediate tumor suppression in cancers other than Ewing sarcoma. The study that identified NELL2 as a repulsion-inducing ligand for Robo3 did not analyze downstream signaling (Jaworski et al., 2015, Science 350, 961-965). It is possible that neurons respond to NELL2 signaling both by reorganization of actin cytoskeletons, which is considered to mediate axon repulsion, and by chromatin remodeling through BAF complexes. Indeed, developmental stage-specific BAF complexes play important roles in mammalian neural development and a large number of mutations of the BAF subunits are found in a variety of human neurological disorders (Son and Crabtree, 2014, American journal of medical genetics Part C, Seminars in medical genetics 166C, 333-349).

NELL2^{high}CD133^{high}EWS-FLI1^{high} and NELL2^{low}CD133^{low}EWS-FLI1^{low} populations were identified in Ewing sarcoma, which display phenotypes consistent with high and low NELL2 signaling, respectively (FIG. 4). Previous studies found that CD133 expression is heterogeneous in Ewing sarcoma tumors and cell lines (Cornaz-Buros et al., 2014, Cancer Res 74, 6610-6622; Jiang et al., 2010, BMC cancer 10, 116; and Suva et al., 2009) and the CD133+ population was suggested to represent the cancer stem cells in Ewing sarcoma (Cornaz-Buros et al., 2014, Cancer Res 74, 6610-6622; and Suva et al., 2009, Cancer Res 69, 1776-1781). Recent studies also found that EWS-FLI1 activity is heterogeneous in Ewing sarcoma cell lines and tumors (Aynaud et al., 2020, Cell reports 30, 1767-1779 e1766; and Franzetti et al., 2017, Oncogene 36, 3505-3514). Described herein is a new link by demonstrating that NELL2, CD133, and EWS-FLI1 positively regulate each other and increase BAF subunits and cell proliferation in Ewing sarcoma (FIG. 5).

Compared with the NELL2^{low}CD133^{low}EWS-FLI1^{low} population, the NELL2^{high}CD133^{high}EWS-FLI1^{high} population displayed higher proliferation, higher tumor sphere formation, higher xenograft tumorigenicity, and higher migration rate, but was more sensitive to chemotherapeutic drug treatment (FIG. 4), suggesting that there are two populations of cells in Ewing sarcoma, each responsible for different aspects of tumor progression, growth vs. chemo-resistance. This is somewhat different from the tumor heterogeneity model established in epithelial carcinomas in which stemness, tumor-initiating capability, migration/invasiveness, and chemotherapy resistance tend to reside in the same or overlapping carcinoma sub-populations. The tumor heterogeneity in Ewing sarcoma and possibly other sarcomas might work differently from that in carcinomas. It will be important to further dissect the roles of the NELL2^{high}CD133^{high}EWS-FLI1^{high} and NELL2^{low}CD133^{low}EWS-FLI1^{low} populations in Ewing sarcoma growth, invasion, drug resistance, and recurrence. While these two populations display contrasting phenotypes, both are dependent on NELL2 (FIG. 4I), supporting the notion that NELL2 signaling as a therapeutic target.

Materials and Methods. Animals. Female 5-6 week old C.B.17SC scid−/− mice were used. Mice were housed in a pathogen-free vivarium. Mice were randomly allocated to treatment groups. Blinding of the researcher measuring tumor size was employed.

Cell Lines. A-673, SK-N-MC, 293, 293T, HeLa, HCT116, IMR-90, and Aska cells were cultured in Dulbecco's modified Eagle's medium (DMEM) supplemented with 10% fetal bovine serum. EW8, TC32, TC71, CHLA-9, ES1, ES2, ES3, ES6, ES7, ES8, RD-ES, RH4, RH5, RD, and RMS13 cells were cultured in RPMI-1640 medium supplemented with 10% fetal bovine serum. SK-NEP-1 and SK-ES-1 cells were cultured in McCoy's 5a medium supplemented with 15% fetal bovine serum. A-673, SK-N-MC, SK-NEP-1, SK-ES-1, RD-ES, RD, RMS13, 293, 293T, HeLa, HCT116, and IMR-90 cells were from ATCC. TC71 cells were from the Coriell Institute for Medical Research. EW8, TC32, and CHLA-9) cells were from Dr. Patrick Grohar. The cell lines were STR-authenticated and were routinely tested for the absence of mycoplasma. Cord blood-derived human mesenchymal stem cells were purchased from Vitro Biopharma (Golden, CO) and were cultured in low-serum MSC-GRO following the manufacturer's procedure.

Transfection and viral infection. Calcium phosphate co-precipitation was used for transfection of 293T cells. Lentiviruses were prepared by transfection in 293T cells following System Biosciences' protocol, and the cells infected with lentiviruses were selected with 2 μg/mL puromycin for 48 hours. The target sequences for shRNAs are as follows: FLI1 C-terminus shRNA, AACGATCAGTAAGAATACAGAGC (SEQ ID NO: 20); luciferase shRNA, GCACTCTGATTGACAAATACGATTT (SEQ ID NO: 21); NELL2 shRNA-1, CGAGAATTTGAGTCCTGGATA (SEQ ID NO: 22); and NELL2 shRNA-2, CCTACTTTGAAGGAGAAAGAA (SEQ ID NO: 23). The following siRNAs were used: human NELL2 siRNA SMARTpool (M-012185-00); Dharmacon), human Robo3 siRNA SMARTpool (M-026504-00; Dharmacon), human Robo3 siRNAs NM_022370 (SASI_Hs01_00099511, SASI_Hs01_00099512, SASI_Hs01_00099513, SASI_Hs01_00099514, SASI_Hs01_00099515 and SASI_Hs01_00099516; MilliporeSigma), human cdc42 siRNA SMARTpool (M-005057-01; Dharmacon), human CD133 siRNA SMARTpool (M-010630-01; Dharmacon), human srGAP1 siRNA SMARTpool (M-026974-00; Dharmacon), srGAP2 siRNA SMARTpool (M-021531-03; Dharmacon), human caveolin-1 siRNA SMARTpool (M-003467-01; Dharmacon), human GDF6 siRNA SMARTpool (M-0330055-01; Dharmacon), and Non-Targeting siRNA Pool #2 (D-0012Jun. 14, 2005; Dharmacon), siRNA transfection of cell lines was performed using Lipofectamine™ RNAiMAX Transfection Reagent (Thermo Fisher).

Protein sample preparation and proteomic analysis. The preparation of secreted protein samples, mass spectrometry analysis, and proteomics data processing were performed (Elzi, D. J., et al. (2016). Genes Cancer 7, 125-135; and Jayabal, P., et al. (2017) Genes Cancer 8, 762-770). A-673 cells were infected with lentiviruses expressing an shRNA against FLI1 C-terminal region or luciferase (control) and were selected with 2 μg/ml puromycin for two days. Cells were washed six times with DMEM without serum. Subsequently, cells were cultured in DMEM without serum for 24 hours and the culture supernatant was harvested. The supernatant was centrifuged, filtered through a 0.45-μm filter (Millipore), and concentrated using a 3,000-Da cut-off Amicon Ultra Centrifugal Filter Units (Millipore). The proteins in each sample were fractionated by SDS-PAGE and visualized by Coomassie blue. Each gel lane was divided into six slices, and the proteins in each slice were digested in situ with trypsin (Promega modified) in 40 mM NH₄HCO₃ overnight at 37° C. The resulting tryptic peptides were analyzed by HPLC-ESI-tandem mass spectrometry on a Thermo Fisher LTQ Orbitrap Velos Pro mass spectrometer. The Xcalibur raw files were converted to mzXML format and were searched against the UniProtKB/Swiss-Prot human protein database (UniProt release 2016_04) using X! TANDEM CYCLONE TPP (2011.12.01.1—LabKey, Insilicos, ISB). Methionine oxidation was considered as a variable modification in all searches. Up to one missed tryptic cleavage was allowed. The X! Tandem search results were analyzed by the Trans-Proteomic Pipeline, version 4.3. Peptide/protein identifications were validated by the Peptide/ProteinProphet software tools (Keller, A., et al. (2002) Anal Chem 74, 5383-5392; and Nesvizhskii, A. I., et al. (2003) Anal Chem 75, 4646-4658). Relative quantification is based on the ratio of the number of the spectra assigned to a protein and the total number of spectra in A-673/luciferase shRNA vs. A-673/EWS-FLI1 shRNA samples.

RNA samples and real-time quantitative RT-PCR. De-identified Ewing sarcoma tumor RNA samples were obtained from the Cooperative Human Tissue Network. Total cellular RNA was isolated using TRIzol reagent (Invitrogen). Reverse transcription was performed using a High Capacity cDNA Reverse Transcription Kit (Thermo Fisher) as per manufacturer's instructions. Quantitative PCR was performed using PowerUp SYBR Green Master Mix (Thermo Fisher) on Applied Biosystems ViiA 7 Real-Time PCR System. Each sample was analyzed in triplicate. The following primers were used: NELL2 forward, 5′-CAGAATGCACTGTTACCTGGA-3′ (SEQ ID NO: 24), NELL2 reverse, 5′-GCTGATCCCAATTCTCTTTCA-3′ (SEQ ID NO: 25); Robo3 forward, 5′-CCGGACGACAGATATTACAACG-3 (SEQ ID NO: 26)′, Robo3 reverse, 5′-GCTGGGTCAATGGTGCTATAG-3′ (SEQ ID NO: 27); NR0B1 forward, 5′-AGGGGACCGTGCTCTTTAAC-3′ (SEQ ID NO: 28), NR0B1 reverse, 5′-CTGAGTTCCCCACTGGAGTC-3′ (SEQ ID NO: 29); NKX2-2 forward, 5′-CAGCGACAACCCGTACAC-3′ (SEQ ID NO: 30), NKX2-2 reverse, 5′-GACTTGGAGCTTGAGTCCTGA-3′ (SEQ ID NO: 31); EZH2 forward, 5′-TGGGAAAGTACACGGGGATA-3′ (SEQ ID NO: 32), EZH2 reverse, 5′-TATTGACCAAGGGCATTCAC-3′ (SEQ ID NO: 33); NPY1R forward, 5′-CCATCGGACTCTCATAGGTTGTC-3′ (SEQ ID NO: 34), NPY1R reverse, 5′-GACCTGTACTTATTGTCTCTCATC-3′ (SEQ ID NO: 35); NGFR forward, 5′-CCTCATCCCTGTCTATTGCTCC-3′ (SEQ ID NO: 36), NGFR reverse, 5′-GTTGGCTCCTTGCTTGTTCTGC-3′ (SEQ ID NO: 37); PAPPA forward, 5′-CAGAATGCACTGTTACCTGGA-3′ (SEQ ID NO: 38), PAPPA reverse, 5′-GCTGATCCCAATTCTCTTTCA-3′ (SEQ ID NO: 39); CD133 forward, 5′-GACCGACTGAGACCCAACAT-3′ (SEQ ID NO: 40), CD133 reverse, 5′-TGGTTTGGCGTTGTACTCTG-3′ (SEQ ID NO: 41); BRG1 forward, 5′-AGCGATGACGTCTCTGAGGT-3′ (SEQ ID NO: 42), BRG1 reverse, 5′-GTACAGGGACACCAGCCACT-3′ (SEQ ID NO: 43); β-actin forward, 5′-AGAGCTACGAGCTGCCTGAC-3′ (SEQ ID NO: 44), β-actin reverse, 5′-AGCACTGTGTTGGCGTACAG-3′ (SEQ ID NO: 45); STAT3 forward, 5′-GGCATTCGGGAAGTATTGTCG-3′ (SEQ ID NO: 46), STAT3 reverse, 5′-GGTAGGCGCCTCAGTCGTATC-3′ (SEQ ID NO: 47); MDR1 forward, 5′-CACGTGGTTGGAAGCTAACC-3′ (SEQ ID NO: 48), MDR1 reverse, 5′-GAAGGCCAGAGCATAAGATGC-3′ (SEQ ID NO: 49); EWS-FLI1 forward, 5′-GGCAGCAGAACCCTTCTTAT-3′ (SEQ ID NO: 50), EWS-FLI1 reverse, 5′-GGCCGTTGCTCTGTATTCTTA-3′ (SEQ ID NO: 51); GAPDH forward, 5′-GGTGTGAACCATGAGAAGTATGA-3′ (SEQ ID NO: 52), GAPDH reverse, GAGTCCTTCCACGATACCAAAG (SEQ ID NO: 53); and EWS forward, 5′-CCACGGATTACAGTACCTATAGC-3′ (SEQ ID NO: 54), EWS reverse, 5′-GGCTGTCCATAGGTTCCATAG-3′ (SEQ ID NO: 55).

Immunoblotting. Immunoblotting was performed (Jayabal, P., et al. (2017) Genes Cancer 8, 762-770). The following antibodies were used: rabbit polyclonal anti-FLI1 (ab15289, Abcam); mouse monoclonal anti-FLAG M2 (F1804, MilliporeSigma): rabbit monoclonal anti-NELL2 (ab181376, Abcam); goat polyclonal anti-PGK1 (sc-17943, Santa Cruz Biotechnology); rabbit polyclonal anti-Robo3 (LS-C345713, LSBio); goat polyclonal anti-Robo3 (PA5-18714, Thermo Fisher Scientific); mouse monoclonal anti-cdc42 (ACD03, Cytoskeleton); rabbit polyclonal anti-Rac1/2/3 (2465, Cell Signaling Technologies); rabbit polyclonal anti-Rho A (2117, Cell Signaling Technologies); goat polyclonal anti-BRG1 (A303-877A, Bethyl Laboratories); rabbit monoclonal anti-BRG1 (49360, Cell Signaling Technologies); rabbit polyclonal anti-BRM (A301-014A-T, Bethyl Laboratories); mouse monoclonal anti-ARID1A/BAF250) (sc-32761, Santa Cruz Biotechnology); rabbit polyclonal anti-BAF170 (A301-039A-T, Bethyl Laboratories); rabbit monoclonal anti-BAF155 (11956, Cell Signaling Technologies); rabbit polyclonal anti-BAF60B (A301-596A-T, Bethyl Laboratories); rabbit polyclonal anti-BAF57 (A300-810A-T, Bethyl Laboratories); rabbit polyclonal anti-BAF53A (A301-391A-T, Bethyl Laboratories); rabbit monoclonal anti-BAF47 (8745, Cell Signaling Technologies); rabbit polyclonal anti-BRD9 (A303-781A-T, Bethyl Laboratories); rabbit monoclonal anti-SS18 (21792, Cell Signaling Technologies); rabbit monoclonal anti-CD133 (64326, Cell Signaling Technologies); rabbit polyclonal anti-srGAP1 (A301-286A-T, Bethyl Laboratories); rabbit polyclonal anti-srGAP2 (GTX130797, GeneTex); mouse monoclonal anti-tubulin (DMIA, Thermo Fisher Scientific); rabbit polyclonal anti-β-actin (4967, Cell Signaling Technologies); mouse monoclonal anti-Rpb1 (2629, Cell Signaling Technologies); rabbit monoclonal anti-cdc6 (3387, Cell Signaling Technologies); rabbit monoclonal anti-caveolin-1 (3267, Cell Signaling Technologies); rabbit polyclonal anti-phospho-caveolin-1 (3251, Cell Signaling Technologies); rabbit monoclonal anti-Src (2123, Cell Signaling Technologies); rabbit monoclonal anti-phospho-Src Family (6943, Cell Signaling Technologies); and rabbit polyclonal anti-GDF6 (NBP1-91934, Novus Biologicals). The following HRP-conjugated secondary antibodies were used: goat anti-rabbit (7074) and goat anti-mouse (7076) (Cell Signaling Technologies); donkey anti-goat (A50)-201P, Bethyl Laboratories).

Immunoprecipitation. Immunoprecipitation was performed (Shiio, Y., and Eisenman, R. N. (2003) Histone sumoylation is associated with transcriptional repression. Proc Natl Acad Sci USA). The following antibodies were used: rabbit monoclonal anti-BRG1 (ab110641, Abcam), rabbit monoclonal anti-BAF57 (33360, Cell Signaling Technologies), and mouse monoclonal anti-FLAG M2 (F1804, MilliporeSigma).

Immunofluorescence. Cells grown on coverslips were fixed with 4% paraformaldehyde for 15 minutes at room temperature, washed with PBS, and permeabilized with 0.2% pre-chilled Triton-X 100/PBS for 5 minutes. The samples were blocked with culture medium for one hour and incubated with the primary antibody for three hours followed by the secondary antibody for one hour. The following primary antibodies were used: rabbit polyclonal anti-NELL2 (NBP1-82527, Novus Biologicals); mouse monoclonal anti-FLAG M2 (F1804, MilliporeSigma); mouse monoclonal anti-CD133 AC133 (130-090-422, Miltenyi Biotec); rabbit polyclonal anti-FLI1 (ab15289, Abcam); and mouse monoclonal anti-CD99 (MS-1633-P0, Thermo Fisher Scientific). The following secondary antibodies were used: Alexa Fluor 488, goat anti-rabbit IgG (A11034, Thermo Fisher Scientific); Alexa Fluor 568, goat anti-rabbit IgG (A11036, Thermo Fisher Scientific); Alexa Fluor 488, goat anti-mouse IgM (A21042, Thermo Fisher Scientific); and Alexa Fluor 594, goat anti-mouse IgG (A11032, Thermo Fisher Scientific). Nuclei were stained with DAPI. DyLight™ 554 Phalloidin (13054, Cell Signaling Technologies) was used to visualize filopodia.

Chromatin immunoprecipitation. Chromatin immunoprecipitation (ChIP) was performed (Carey, M. F., et al. (2009). Chromatin immunoprecipitation (ChIP). Cold Spring Harbor protocols 2009, pdb prot5279) using rabbit polyclonal anti-FLI1 antibody (ab15289, Abcam), rabbit monoclonal anti-BRG1 antibody (ab110641, Abcam), or control rabbit IgG (ab37415, Abcam). The primer sequences used for ChIP are as follows: NELL2 forward, 5′-CTCTCTCTCTCTCTCTAACCATCTC-3′ (SEQ ID NO: 56), NELL2 reverse, 5′-TCTCTGGGACCAGCATACA-3′ (SEQ ID NO: 57); FOXO1 forward, 5′-GGAAGAGGTTCCCACGGAGGGCAT-3′ (SEQ ID NO: 58), FOXO1 reverse, 5′-CCGGCGACACTTTGTTTACT-3′ (SEQ ID NO: 59); GLI-1 forward, 5′-AGAGCCTGGGGGTGAGACAT-3′ (SEQ ID NO: 60), GLI-1 reverse, 5′-GCCTCTTCAACTTAACCGCATGA-3′ (SEQ ID NO: 61); NR0B1 forward, 5′-GTTTGTGCCTTCATGGGAAATGGTTATTC-3′ (SEQ ID NO: 62): NR0B1 reverse, 5′-CTAGTGTCTTGTGTGTCCCTAGGG-3′ (SEQ ID NO: 63); GAPDH forward, 5′-TCCTCCTGTTTCATCCAAGC-3′ (SEQ ID NO: 64); GAPDH reverse, 5′-TAGTAGCCGGGCCCTACTTT-3′ (SEQ ID NO: 112); CD133 P1 forward, 5′-GAACTGCGGGGAGAGCGTGGTG-3′ (SEQ ID NO: 65); P1 reverse, 5′-TCCCCGAGAGCGAGTCCGAAGTC-3′ (SEQ ID NO: 66); CD133 P2 forward, 5′-CGACCACAGCGGGAGTAG-3′ (SEQ ID NO: 67); P2 reverse, 5′-GCGAGAGGCTGGGAAGGT-3′ (SEQ ID NO: 68); CD133 P3 forward, 5′-GACCGGACAACAAAGAGGAG-3′ (SEQ ID NO: 69); P3 reverse, 5′-CTCCAGACACGGGCTTTC-3′ (SEQ ID NO: 70); CD133 P4 forward, 5′-CCGCCCCGCCGCTCATTC-3′ (SEQ ID NO: 71); P4 reverse, 5′-GCTTCCCCGCCCTTTACCTC-3′ (SEQ ID NO: 72); CD133 P5 forward, 5′-TATGGCTTTATGCTGTTTTTCAA-3′ (SEQ ID NO: 73); P5 reverse, 5′-CTCATCCCGGCCGCATTAGAC-3′ (SEQ ID NO: 74); CD133 P6 forward, 5′-GTGCTTCCTGCTCCTCTTC-3′ (SEQ ID NO: 75); P6 reverse, 5′-GCTAGCAAGATCCTCCAAACA-3′ (SEQ ID NO: 76); and EWS forward, 5′-TCCCAAAGTGCTGGGATTAC-3′ (SEQ ID NO: 77), EWS reverse, 5′-TCTCATGGTTTCCCTTTCTAGC-3′ (SEQ ID NO: 78). The EWS primers amplify an 87 bp sequence approximately 3 kb from the EWS transcription start site. The genomic locations of the primers are: EWS forward primer, chromosome 22, 29265034-29265054 and EWS reverse primer, chromosome 22, 29265098-29265120.

GST pull-down assays. The active form of cdc42, Rac, and Rho A was analyzed by pull-down of whole cell lysate with GST-PAKI (active cdc42 and Rac) and GST-RBD (active Rho A) followed by immunoblotting for cdc42, Rac, and Rho A as described (Ren, X. D., et al. (1999) EMBO J 18, 578-585).

Gel filtration chromatography. Nuclear extract was prepared as described (Carey, M. F., et al. (2009) Dignam and Roeder nuclear extract preparation. Cold Spring Harbor protocols 2009, pdb prot5330). The extract (5 mg) was loaded onto HiPrep 16/60 Sephacryl S-400 HR column (GE Healthcare) equilibrated with a gel filtration buffer (150 mM NaCl, 20 mM Hepes-KOH, 10 mM KCl, 1 mM MgCl2, 1 mM EDTA, 1 mM EGTA, 1 mM DTT, pH 7.5). Proteins were eluted at 1 ml/min and 1-ml fractions were collected. Every fourth fraction was concentrated and analyzed by immunoblotting.

Phalloidin dot blot assays. The BAF complexes were immuno-purified from nuclear extract using anti-BRG1 or anti-BAF57, (for endogenous BAF) or anti-FLAG antibody (for FLAG-BRG1-containing BAF) and were spotted onto a nitrocellulose membrane (sample amount normalized by cell number). The membrane was allowed to dry at room temperature for 30 minutes. The membrane was rinsed with TBST (150 mM NaCl, 20 mM Tris, pH7.4, 0.05% Tween-20), blocked with 5% BSA in TBST, and incubated with Biotin-XX Phalloidin (Biotium) either overnight at 4° C. or for two hours at room temperature followed by three washes with TBST. The membrane was incubated with High Sensitivity Streptavidin-HRP (21130, Pierce/Thermo Fisher) for one hour and was washed three times with TBST, followed by detection with ECL reagent.

Cell proliferation and xenograft tumorigenicity assays. Anchorage-dependent cell proliferation was assessed by IncuCyte live-cell imaging system (Essen BioScience). The IncuCyte system monitors cell proliferation by analyzing the occupied area (% confluence) of cell images over time. At least four fields from four wells were assayed for each experimental condition. The cell seeding density was 2000 cells per well in a 96-well plate. For each assay, biological replicates were performed to confirm the reproducibility of results. Anchorage-independent cell proliferation was evaluated by soft agar colony formation assays. A-673 cells were infected with lentiviruses expressing shRNAs against NELL2 or scrambled shRNA and were selected with 2 μg/ml puromycin. Four days after infection, 4×10³ cells were plated in soft agar. The soft agar cultures were composed of two layers; a base layer [4 ml in a 60-mm dish; DMEM/10% fetal bovine serum/0.6% noble agar (A5431, MilliporeSigma)/penicillin/streptomycin] and a cell layer (2 ml in a 60-mm dish; DMEM/10% fetal bovine serum/0.3% noble agar/penicillin/streptomycin). Colonies were grown for three weeks and counted. Colonies (>50 cells) were scored by randomly counting 10 fields per dish. Sphere formation assays were done (Dasgupta, A., et al. (2017) Oncotarget 8, 77292-77308) using ultra-low attachment 6-well plates (Corning: seeding density 1×10⁴ cells/well) and DMEM/F-12 medium supplemented with B27, human recombinant epidermal growth factor (20 ng/ml), and basic fibroblast growth factor (20 ng/ml). For xenograft tumorigenicity assays, cells were subcutaneously injected into the flanks of SCID mice (2×10⁶ cells/injection, five mice/group). Tumor growth was monitored weekly using a caliper. While it is not possible to predict the effect size, the sample size of five mice per group was chosen based on experience with xenograft experiments. Mice were randomly allocated to treatment groups. Blinding of the researcher measuring tumor size was employed.

Migration assays. Migration assays were performed in modified Boyden chambers (Corning® Transwell R, 8 μm pore size, MilliporeSigma). Cells were seeded at 5×10⁴ cells/200 μl serum free-DMEM/F-12 medium in the upper chamber. The lower chamber was filled with DMEM/F-12 supplemented with 10% FBS. After 20-hour incubation, the membrane was gently removed from the chamber and the cells on the upper surface were removed using cotton swabs. Cells on the lower surface that migrated through the membrane were fixed with 50% methanol and stained with 0.1% crystal violet. The migrated cells were counted from eight randomly chosen fields.

Flow cytometry. Cells were trypsinized, washed with FACS wash buffer (PBS, 0.5% BSA, 2 mM EDTA), and incubated with PE-conjugated human CD133/1 antibody (clone AC133, Miltenyi Biotec; 1:100 in FACS wash buffer) for 20 minutes at 4° C. Cells were washed three times with FACS wash buffer and the CD133^{high} and CD133^{low} cell populations were sorted by using BD FACSAria (Becton Dickinson). The FACSDiva 6.1.3 software (Becton Dickinson) was used for sample analysis.

Dissociation of a patient-derived xenograft tumor. A patient-derived xenograft tumor was washed and dissociated into single-cell suspensions using the tumor dissociation kit (Miltenyi Biotec 130-095-929) and gentleMACS Octo-dissociator (Miltenyi Biotec 130-096-427), following the manufacturer's protocol. Dissociated cells were cultured in DMEM/F-12 medium supplemented with 10% FBS.

Statistical analysis. Statistical analyses were performed with Prism (GraphPad Software) with a two-tailed Student's t test. Data are expressed as mean±SEM. The results were considered significant when p<0.05. The number of replicates, independent samples, and animals is indicated in the figure legends.

### Example 2: A Method to Target NELL2 Signaling in Tumors

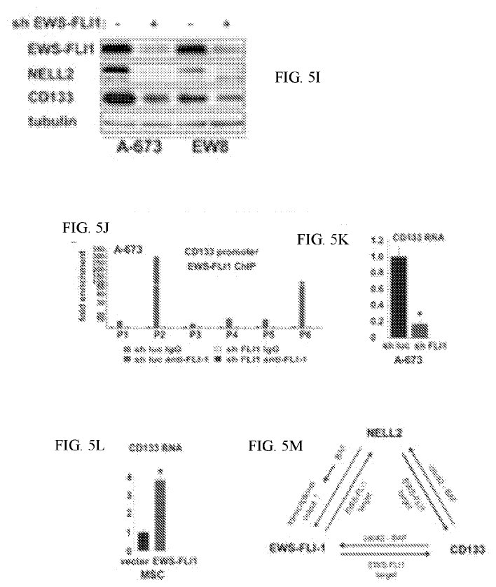

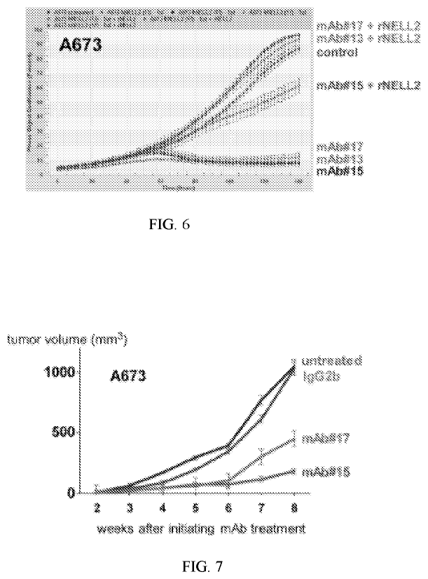

Ewing sarcoma depends on the autocrine signaling mediated by a secreted protein called NELL2. Mouse monoclonal antibodies (mAbs) were generated against the EGF-like repeat domain of NELL2 to neutralize the protein. Out of the several generated mAbs, three clones (#13, 15 (SEQ ID NOs: 89 and 99), 17 (SEQ ID NOs: 14 and 4) were identified that strongly inhibited Ewing sarcoma cell proliferation (FIG. 6: mAbs used at 1 μg/ml) and which demonstrated stable mAb production. Growth arrest by NELL2 mAbs was rescued by adding recombinant NELL2 to the culture medium (FIG. 6), indicating on-target toxicity. These NELL2 mAbs also demonstrated therapeutic potential in vivo (20 mg/kg i.p. injection, twice weekly) by efficiently inhibiting xenograft tumor growth of A673 cells in SCID mice (FIG. 7). Thus, described herein are methods to treat Ewing sarcoma using anti-NELL2 monoclonal antibodies. In addition to Ewing sarcoma, it was found that neuroblastoma, medulloblastoma, and glioblastoma also depend on NELL2 signaling, suggesting that these cancers can also be treated by administration of anti-NELL2 monoclonal antibodies.

A bacterial expression vector for His- and SBP (streptavidin-binding peptide)-double-tagged NELL2 EGF-like repeats (residue 397-637) was made and ˜2 mg of EGF-like repeats were isolated by tandem affinity purification followed by TEV protease cleavage. Mice were immunized with NELL2 EGF-like repeats and hybridoma clones were generated and screened by immunogen ELISA. Twenty-five ELISA-positive hybridoma clones were further screened for growth inhibition of A673 Ewing sarcoma cells, which identified three clones (#13, 15, 17) that strongly inhibited Ewing sarcoma cell proliferation (FIG. 6; mAbs used at 1 μg/ml) and demonstrated stable mAb production. Growth arrest by NELL2 mAbs was rescued by adding recombinant NELL2 to the culture medium (FIG. 6), indicating on-target toxicity. These NELL2 mAbs (20 mg/kg i.p. injection, twice weekly) efficiently inhibited xenograft tumor growth of A673 cells in SCID mice (FIG. 7).

1. An isolated antibody comprising a light chain variable region and a heavy chain variable region, wherein the light chain variable region comprises a complementarity determining region light chain 1 (CDRL1) amino acid sequence of SEQ ID NO: 1; a determining region light chain 2 (CDRL2) amino acid sequence of SEQ ID NO: 2; and a determining region light chain 3 (CDRL3) amino acid sequence of SEQ ID NO: 3; and wherein the heavy chain variable region comprises a complementarity determining region heavy chain 1 (CDRH1) amino acid sequence of SEQ ID NO: 11; a complementarity determining region heavy chain 2 (CDRH2) amino acid sequence of SEQ ID NO: 12; and a complementarity determining region heavy chain 3 (CDRH3) amino acid sequence of SEQ ID NO: 13.

2. The isolated antibody of claim 1, comprising a light chain variable region amino acid sequence of SEQ ID NO: 4.

3. The isolated antibody of claim 1, comprising a heavy chain variable region amino acid sequence of SEQ ID NO: 14.

4. An isolated antibody comprising a light chain variable region amino acid sequence of SEQ ID NO: 4 and a heavy chain variable region amino acid sequence of SEQ ID NO: 14.

5. The isolated antibody of claim 4, wherein a light chain variable region has an amino acid sequence that is at least 90% identical to amino acid sequence of SEQ ID NO: 4.

6. The isolated antibody of claim 4, wherein a heavy chain variable region has an amino acid sequence that is at least 90% identical to amino acid sequence of SEQ ID NO: 14.

7. The isolated antibody of claim 1, wherein the antibody is recombinantly engineered, chimerized, or humanized.

8. The isolated antibody of claim 1, wherein the antibody is a Fab, an Fab′, an F(ab′)2, a Fv, a scFv, a diabody or fragments thereof.

9. The isolated antibody of claim 1, wherein the antibody binds to human NELL2.

10.-13. (canceled)

14. The isolated antibody of claim 1, wherein the antibody inhibits binding of human NELL2 to human Robo3.

15. The isolated antibody of claim 1, wherein the antibody is a monoclonal antibody.

16. The isolated antibody of claim 1, wherein the antibody is a single chain antibody.

17. The isolated antibody of claim 1, wherein the antibody is linked to a detectable label.

18. The isolated antibody of claim 1, wherein the antibody is a monovalent or a bivalent antibody.

19. The isolated antibody of claim 1, wherein the antibody is an IgG, an IgM, an IgA antibody or antigen binding fragment thereof.

20. (canceled)

21. (canceled)

22. A composition comprising the isolated antibody of claim 1, and at least one pharmaceutically acceptable carrier or diluent.

23. (canceled)

24. A method of treating a NELL2 positive cancer in a subject in need, the method comprising administering to the subject having cancer a therapeutically effective amount of the isolated antibody of claim 1.

25. The method of claim 24, wherein the cancer is Ewing sarcoma, neuroblastoma or a brain cancer.

26. The method of claim 25, wherein the brain cancer is glioblastoma or medulloblastoma.

27.-44. (canceled)

45. An isolated antibody comprising a light chain variable region and a heavy chain variable region, wherein the light chain variable region comprises a complementarity determining region light chain 1 (CDRL1) amino acid sequence of SEQ ID NO: 96; a determining region light chain 2 (CDRL2) amino acid sequence of SEQ ID NO: 97; and a determining region light chain 3 (CDRL3) amino acid sequence of SEQ ID NO: 98; and wherein the heavy chain variable region comprises a complementarity determining region heavy chain 1 (CDRH1) amino acid sequence of SEQ ID NO: 86; a complementarity determining region heavy chain 2 (CDRH2) amino acid sequence of SEQ ID NO: 87; and a complementarity determining region heavy chain 3 (CDRH3) amino acid sequence of SEQ ID NO: 88.

46.-53. (canceled)
